SEQUENCE LISTING (10 sequences)

SEQ ID NO: 1 - DNA, 40 bp, Artificial
  H9 IL-2 Superkine FWD
ttattttattttatttaaccggagccatgggagaattcgc

SEQ ID NO: 2 - DNA, 43 bp, Artificial
  H9 IL-2 Superkine REV
ggaggagggcggccgcttacctaagttagtgttgagatgatgc

SEQ ID NO: 3 - DNA, 40 bp, Artificial
  pCellFree_G03_IL2 backbone, first section FWD
gcgaattctcccatggctccggttaaataaaataaaataa

SEQ ID NO: 4 - DNA, 40 bp, Artificial
  pCellFree_G03_IL2 backbone, first section REV
ttgctacaggcatcgtggtgtcacgctcgtcgtttggtat

SEQ ID NO: 5 - DNA, 40 bp, Artificial
  pCellFree_G03_IL2 backbone, second section FWD
ataccaaacgacgagcgtgacaccacgatgcctgtagcaa

SEQ ID NO: 6 - DNA, 43 bp, Artificial
  pCellFree_G03_IL2 backbone, second section REV
gcatcatctcaacactaacttaggtaagcggccgccctcctcc

SEQ ID NO: 7 - DNA, 420 bp, Artificial
  IL-2 of Superkine (Super2)
ggagccatgggagaattcgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcagagcaaaaactttcacttcgatcccagggacgtcgtcagcaatatcaacgtattcgtcctggaactaaagggatctgaaacaacattcatgtgtgaatatgctgatgagacagcaaccattgtagaatttctgaacagatggattaccttttgtcaaagcatcatctcaacactaactcat

SEQ ID NO: 8 - protein, 8 aa, Artificial
  RGD containing peptide
GGGGRGDY

SEQ ID NO: 9 - DNA, 2115 bp, Artificial
  Plasmid with IL-2 Superkine (Super2)
gaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaggatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctagttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 10 - protein, 138 aa, Artificial
  IL-2 Superkine (Super2)
MGEFAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTH

(19) United States
(12) Patent Application Publication
(10) Pub. No.: US 2022/0304928 A1
(43) Pub. Date: Sep. 29, 2022

(54) PROTEIN PRODUCING NANOLIPOSOMES AND USES THEREOF

(71) Applicant: The Regents of the University of California, Oakland, CA (US)

(72) Inventors: Manish J. BUTTE, Los Angeles, CA (US); Mohammad Mahdi HASANI-SADRABADI, Los Angeles, CA (US); Fatemeh S. MAJEDI, Los Angeles, CA (US)

(73) Assignee: The Regents of the University of California, Oakland, CA (US)

(21) Appl. No.: 17/639,803

(22) PCT Filed: Sep. 18, 2020
(86) PCT No.: PCT/US20/51365

§ 371 Date: Mar. 2, 2022

Related U.S. Application Data
(60) Provisional application No. 62/902,883, filed on Sep. 19, 2019.
(60) Provisional application No. 62/902,390, filed on Sep. 18, 2019.

(51) Int. Cl. A61K 9/127; A61K 41/00; A61K 38/20; A61K 9/48; A61K 9/50; A61P 35/00; A61P 37/06; A61K 35/17; A61K 48/00; A61P 37/08; A61P 31/04; A61P 17/02; A61P 9/10

(52) U.S. Cl. CPC A61K 9/127; A61K 41/0042; A61K 38/2013; A61K 9/4866; A61K 9/5015; A61P 35/00; A61P 37/06; A61K 35/17; A61K 48/0025; A61K 9/1277; A61P 37/08; A61P 31/04; A61P 17/02; A61P 9/10

(57) ABSTRACT

Synthetic liposomal nanoparticles comprising a cell-free transcription and translation machinery, a plasmid encoding a cytokine, and a regulatable caged ATP molecule, as well as microparticles encasing the synthetic liposomal nanoparticles and methods of making and using the synthetic liposomal nanoparticles, are described herein. These liposomal nanoparticles may be used for the controlled release o a cytokine within a localized environment of, for example a tumor, as part of a therapeutic treatment of cancer, or for localized treatment at a focus of interest of an autoimmune disease, an allergic reaction or hypersensitivity reaction, a localized site of an infection or infectious disease, a localized site of an injury or other damage, a transplant or other surgical site, or a blood clot. Further, microparticles produced by encapsulating hundreds of liposomal nanoparticles, and their therapeutic uses, are also described.

### CROSS-REFERENCE TO RELATED APPLICATIONS

This application claims priority to U.S. Provisional Patent Application 62/902,390, filed Sep. 18, 2019, and U.S. Provisional Patent Application 62/902,883, filed Sep. 19, 2019, which are incorporated by reference herein in their entirety.

### STATEMENT OF GOVERNMENT INTEREST

The invention described herein was supported in whole or in part by grants from The National Institutes of Health (Grant Nos. R01 GM110482 and 1R56DE029157). The government has certain rights in the invention. In addition, interleukin-2 used for this study was provided by the BRB Preclinical Repository of the National Cancer Institute, Frederick, Md., USA.

### FIELD OF INTEREST

This disclosure relates to synthetic liposomal nanoparticles comprising a cell-free transcription and translation machinery, a plasmid encoding a cytokine, and a regulatable caged adenosine triphosphate (ATP) molecule. These liposomal nanoparticles may be used for the controlled release of a cytokine within a localized environment of a tumor, e.g., as part of a therapeutic treatment of cancer, or for localized treatment at a focus of interest of an autoimmune disease or an allergic reaction, a localized site of an infection or infectious disease, a localized site of an injury or other damage, a transplant or other surgical site, or a blood clot. They may also be used for the controlled release of a cytokine for the regulation of immunity in general and for other therapeutic uses.

### BACKGROUND

Therapeutic modulation of immunity has made significant headway in the fight against cancer. However, global immunomodulation results in systemic adverse effects including severe inflammation, provocation of autoimmunity, and susceptibility to infection.

Nanoliposomes are bilayer lipid vesicles. These are nano versions of liposomes, that may include cell-specific targeting, encapsulated effector biomolecules, and additionally may be encapsulated into larger microparticles. These larger microparticles may include a lipid coating and or targeting molecules. A number of groups have developed polymeric carriers, such as microparticles, nanoliposomes, or micelles for delivering biomolecules. Such particles/nanoliposomes have be used to modulate the immune system by delivering antigens, vaccines, adjuvants, and drugs. The focus of prior work has mainly been on enhancing stability or solubility of the delivered molecules, little has been done on sustained release of biomolecules, such as cytokines, and its impact on therapeutic efficacy.

Cytokines influence the proliferation and differentiation of cultured, primary T cells. Augmentation and engineering of immune responses have major applications in combating cancers, including solid tumor cancers.

IL-2 is a cytokine that plays the major role in activation and expansion of helper and cytotoxic T cells (CTLs) to fight infections and cancer. IL-2 also helps activate natural killer cells for fighting viruses and cancer. Unfortunately, systemic delivery of IL-2 has been shown to be inefficient and has additional limitations including continuous secretion eliciting non-specific immune response.

A tumor, whether benign or malignant, is caused by abnormal growth of cells or a tissue. Cancer is an abnormal and malignant state in which uncontrolled proliferation of one or more cell populations interferes with normal biological functioning. Standard treatments for cancer include surgery, chemotherapy, and radiation therapy. T cell immunotherapy is a promising approach for cancer. However, significant challenges hamper its therapeutic potential, including insufficient activation, delivery, and clonal expansion of T cells into the tumor environment. Even non-cancerous tumors may pose significant health challenges, such as when they are located at treatment site that is difficult to access or when they chronically recur. 91% of deaths from cancer occur due to solid tumors, over 1000 deaths per day, highlighting a profound unmet need for new therapies. Solid tumors elude clearance by T cells due to a variety of immunosuppressive features of the tumor microenvironment. TGF-β made in the tumor milieu promotes development of regulatory T cells, which suppress cytotoxic responses, but TGF-β cannot easily be suppressed globally because of autoimmune and other side effects. Cytotoxic effector functions of intratumoral T cells are weakly activated, but global T-cell activation cannot be pursued due to adverse effects like cytokine storm.

Some infectious and non-infectious medical conditions exist, at least initially, in localized environments within the body. For example, these types of diseases and conditions are often difficult to treat without systemic exposure to therapeutic agents, which may have significant side effects.

Some autoimmune diseases (e.g., rheumatoid arthritis, juvenile dermatomyositis, psoriasis, psoriatic arthritis, sarcoidosis, lupus, Crohn's disease, eczema, vasculitis, ulcerative colitis, multiple sclerosis, type I diabetes) may present with at least some localized symptoms or symptoms in a particular system of the body, but treatment options may leave the patient having to choose between alleviating one or more symptoms (e.g., use of a non-steroidal anti-inflammatory drug [NSAID] or an antihistamine or a dermatological ointment or cream providing limited relief of a given symptom) or systemic exposure of the entire body to a more aggressive treatment (e.g., methotrexate) with a concomitant increase in potentially dangerous side effects. Likewise, some infectious diseases (e.g., shingles) or initially localized infections (e.g., methicillin-resistant Staphylococcus aureus [MRSA] infection) may have few treatment options or may require the use of more aggressive systemic treatments. In addition, traumatic injury, chronic damage (e.g., osteoarthritis), surgery, or a blood clot may necessitate the use of more aggressive systemic treatments, notwithstanding the limited location of the injury or surgical site. Furthermore, the concern over potential rejection of a transplant (e.g., a transplanted organ) necessitates aggressive systemic treatments with immune suppression drugs, often with significant side effects, also notwithstanding the limited location of the transplant site.

Cytokine therapy, checkpoint inhibitor therapy, and other forms of immunomodulation have proven exceptionally potent in the fight against cancer. However, globally altering the immune system can lead to adverse systemic effects, including severe inflammation, autoimmunity, and increased susceptibility to infection.

Despite recent successes in cancer immunotherapies that emphasize high therapeutic potency in treating patients with progressive tumors, significant challenges, including insufficient activation and eventual exhaustion of effector T cells as well as suppression of their effector responses in the tumor microenvironment; and inadequate ability to expand tumor-specific T cell ex vivo hinder the potential of T cell therapies, especially in solid tumors. Most of the current immunotherapy approaches aim to facilitate T cells to fight tumors and provoke their infiltration. Some of the commonly used strategies include blocking inhibitory receptors such as anti PD-1 and anti CTLA-4 while others include evoking cytotoxic T lymphocyte (CTL) responses such as chimeric antigen receptor (CAR)-T cell therapies and adoptive cell transfer (ACT) approaches. Despite their revolutionary approaches for hematopoietic cancers, potency of these methods and the need for expanding tumor-specific T cells is a need not yet satisfactorily met for solid tumor therapy. One of the major flaws associated with checkpoint inhibitor therapies (CPI) and chemokine therapies such as IL-2 or IL-12 that hampers their clinical translation is their administration route and all the immune-related adverse events that are affiliated with it. In this regard few attempts have been made towards making the deliveries more targeted. Nanogel “backpacks” have demonstrated the release of cytokines to T cells. However, the continual release of cytokines risks systemic exposure, side effects, and compromise of a limited supply of cytokine. Collagen-binding domain fused to IL-12 is another example that emphasizes the impact of tumor targeting and prolongation of cytokine release in the tumor stroma. Though, the IV administration in this case especially puts patients with cardiovascular disease at risk. The other matter in these systems is that the rate of release of cytokines is not well controlled. It has been shown that the rate at which cytokines are delivered to CD8+ T cells impacts their differentiation and effector functionality. To tackle the issues related to ACT and improve ex vivo activation and expansion of tumor-reactive T cells, antigen-presenting cell (APC) mimetic scaffolds have been developed that show polyclonal expansion of T cells. Yet they lack the ability to manipulate tumor microenvironment so that it favors formation of tumor fighting T cells.

Another challenge that tumors face is the presence of T regulatory cells (Tregs). Transforming growth factor β (TGF-β) is known to be a key factor in the induction of Tregs from helper T cells drawn to the tumor, which then promotes cancer growth and metastasis. TGF-β also potently inhibits cytotoxic T cells in the tumor microenvironment and has, therefore, become an exciting target in the enhancement of immunotherapy. However, systemic TGF-β inhibition in preclinical models has shown major adverse effects on the cardiovascular, gastrointestinal, and skeletal systems, owing to the pleiotropic effects that TGF-β plays across the body. The release of TGF-β inhibitors by injected nanoliposomes has been shown to reduce metastases but has not shown a local impact in regulatory T cells. Moreover, mechanical stiffness of the niche in which T cells home and face antigens makes a difference on their fate.

Thus, there remains an unmet need for compositions and methods of treatment of cancers and other tumors, for example, but not limited to, treatment of benign or malignant solid tumors. A major gap in treatment exists, wherein there is an inability to provide local factors where most needed in the treatment of solid tumors, while avoiding systemic exposure to immunomodulatory agents.

Similarly, there remains an unmet need for compositions and methods of treatment of localized symptoms of, for example, but not limited to, infectious and non-infectious medical conditions, injuries, damage, surgery, and transplant. A major gap in treatment exists, wherein there is an inability to provide local factors and other treatments where most needed in the treatment of localized symptoms, while avoiding systemic exposure to immunomodulatory agents.

Biosynthesis of proteins in vivo from nanoparticles acting as artificial “cells” allows for the production of cytokines with a number of features not attainable by conventional biological systems: tunable initiation to eliminate the systemic toxicity of basal/continuous expression; controlled release to locally focus the site of the cytokines' activity; and targeting to attach the nanoparticles to T cells. Provided herein, is an in vivo synthesis of the cytokine interleukin 2 (IL-2) from artificial nanoparticles that may be targeted to the area of a cancer, tumor, infection, or other localized symptom, disease, or medical condition and used to tune T cell fate, and thereby promoting clearance of the cancer, tumor, infection, or other localized symptom, disease, or medical condition.

### SUMMARY

The in situ biosynthesis of proteins allows for a number of features not attainable by other approaches to deliver proteins exogenously: tunable initiation to eliminate the systemic toxicity of basal/continuous expression and controlled release to locally focus the site of the cytokines' activity. Provided herein is a new approach for in situ synthesis of the interleukin-2 (IL-2) at the site of immunological action to tune T cell fate locally and to augment safely the immune response in order to promote clearance of cancer, e.g., via local synthesis of immune therapies, thereby significantly reducing systemic toxicity.

In some aspects, disclosed herein is a synthetic nanoliposome comprising (a) a cell-free transcription and translation system; and (b) a plasmid comprising a nucleic acid encoding a protein. In a related aspect, the synthetic nanoliposome further comprises: (c) a photoactivatable-caged adenosine triphosphate (ATP). In a related aspect, the photoactivatable-caged ATP comprises an ultraviolet (UV)-caged ATP or an infrared (IR)-caged ATP. In a related aspect, the size of the nanoliposome comprises about 100-400 nm. In another related aspect, the size of said plasmid comprises about 3000 bp-7000 bp. In another related aspect said plasmid comprises an expression plasmid. In another related aspect, the protein comprises a therapeutic or diagnostic protein. In another related aspect, the protein comprises a cytokine, a chemokine, a therapeutic or diagnostic antibody or fragment thereof, an antigen-binding protein, a Fc fusion protein, an anticoagulant, an enzyme, a hormone, or a thrombolytic. In another related aspect, the cytokine comprises an interleukin. In another related aspect, the interleukin comprises an IL-2, an IL-4, an IL-10, an IL-12, or an IL-15. In yet another related aspect, a cytokine may include a human cytokine. In still another related aspect, an IL-2 cytokine comprises an IL-2 superkine. In another related aspect, the IL-2 superkine (Super2) is encoded by the nucleotide sequence as set forth in SEQ ID NO: 7 and has the amino acid sequence set forth in SEQ ID NO:10. In another related aspect, the plasmid comprises a pCellFree_G03_H9 plasmid expressing the IL-2 superkine. In yet another related aspect, the plasmid has the nucleotide sequence as set forth in SEQ ID NO: 9.

In some aspects, disclosed herein is a microparticle comprising at least one synthetic nanoliposome, the at least one nanoliposome comprising (a) a cell-free transcription and translation system; and (b) a plasmid comprising a nucleic acid encoding a protein. In a related aspect, the synthetic nanoliposome further comprises: (c) a photoactivatable-caged adenosine triphosphate (ATP). In a related aspect, the photoactivatable-caged ATP comprises an ultraviolet (UV)-caged ATP or an infrared (IR)-caged ATP. In a related aspect, microparticles comprise about 400 nanoliposomes. In another related aspect, microparticles further comprise superparamagnetic iron oxide nanoparticles (SPION, SION). In another related aspect, microparticles further comprise upconversion nanoparticles (UCNPs). In yet another related aspect, microparticles may comprise alginate; or alginate-heparin. In still another related aspect, microparticles comprising alginate or alginate-heparin further comprise a lipid membrane coating. In a related aspect, the lipid membrane comprises 1-palmitoyl-2-oleoyl-sn-glycero-3-phosphocholine (POPC; IUPAC [(2R)-3-hexadecanoyloxy-2-[(Z)-octadec-9-enoyl]oxypropyl]2-(trimethylazaniumyl)ethyl phosphate). In a related aspect, the microparticle further comprises at least two types of synthetic nanoliposomes, each type of nanoliposome comprising (a) a cell-free transcription and translation system; and (b) a plasmid comprising a nucleic acid encoding a protein specific to the type of synthetic nanoliposome.

In some aspects, disclosed herein is a method of regulating an immune response at a focus of interest in a subject in need, said method comprising: (a) administering a synthetic nanoliposome or a microparticle comprising a synthetic nanoliposome to said subject, at or adjacent to said focus of interest, said synthetic nanoliposome comprising: a cell-free transcription and translation system; and a plasmid comprising a nucleic acid encoding a protein; (c) expressing said therapeutic or diagnostic protein; and (d) releasing said protein at or adjacent to said focus of interest, said regulating the immune response comprising: increasing proliferation of cytotoxic T cells; increasing proliferation of helper T cells; maintaining the population of helper T cells at the site of said focus of interest; activating cytotoxic T cells at the site of said focus of interest; or any combination thereof. In a related aspect, the synthetic nanoliposome further comprises a photoactivatable-caged adenosine triphosphate (ATP) and prior to the step of expressing said therapeutic or diagnostic protein, said site of administration is exposed to a light source to photoactivate said photoactivatable-caged ATP.

In a related aspect, the photoactivatable-caged ATP comprising an ultraviolet (UV)-caged ATP or an infrared (IR)-caged ATP, and said administering comprising administering of said synthetic nanoliposome or said microparticle through a catheter comprising a UV or IR light. In another aspect, said administration comprises injection of said synthetic nanoliposome or said microparticle. In still another aspect, the injection comprises subcutaneous injection.

In another related aspect, the method further comprising a step of administering activated T cells to said subject. In another aspect, administering of said activated T cells is concomitant with administering said synthetic nanoliposome or said microparticle or is prior to or after administering said synthetic nanoliposome or said microparticle. In yet another aspect, the photoactivatable-caged ATP comprising an ultraviolet (UV)-caged ATP or an infrared (IR)-caged ATP, and said administering of said activated T cells is prior to or after exposing the site to UV or IR light.

In a related aspect, said focus of interest comprises a solid tumor. In another related aspect, said focus of interest comprises an autoimmune-targeted or symptomatic focus of an autoimmune disease; a reactive focus of an allergic reaction or hypersensitivity reaction, a focus of infection or symptoms of a localized infection or infectious disease; an injury or a site of chronic damage; a surgical site; a site of a transplanted organ, tissue, or cell; or a site of a blood clot causing or at risk for causing a myocardial infarction, ischemic stroke, or pulmonary embolism. In a related aspect, administration comprises injection of said nanoliposome or a microparticle. In a related aspect, administration comprises administration of said nanoliposome or microparticle through a catheter comprising a UV or IR light. In a related aspect, where said focus of interest comprises a blood clot causing or at risk for causing a myocardial infarction, an ischemic stroke, or a pulmonary embolism, the administration is at or adjacent to the site of the blood clot together with angioplasty or another clot removal treatment. In another related aspect, said site comprises an area adjacent to said tumor. In another related aspect, said site comprises an area at or adjacent to said autoimmune-targeted or symptomatic focus of an autoimmune disease; said reactive focus of an allergic reaction or hypersensitivity reaction; said focus of infection or symptoms or a localized infection or an infectious disease; said injury or site of chronic damage; said surgical site; said site of a transplanted organ, tissue, or cell; or said site of a blood clot causing or at risk for causing a myocardial infarction, ischemic stroke, or pulmonary embolism.

In a further related aspect, said method further comprises a step of administering activated T cells to said subject. In a related aspect, the administration of said activated T cells is prior to or after administering said nanoliposome or said microparticle. In another related aspect, administration of said activated T cells is prior to or after exposing the site to UV light.

In some aspects, microparticles can be targeted to and be bound to lymphocytes such as T cells, or other blood components, during leukapheresis or other blood cell purification procedure and infused into the patient. Such targeting binding of microparticles to lymphocytes or other cell types after administration to the body or during a leukapheresis procedure or other ex vivo procedure provides the therapeutic protein in association with a cell type to effect its desired regulation of an immune response when activated by UV light.

In a related aspect, said tumor comprises a solid tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In yet another aspect, the method further comprises (a) administering two or more types of microparticles, each type of microparticle comprising a synthetic nanoliposome comprising (i) a cell-free transcription and translation system; and (ii) a plasmid comprising a nucleic acid encoding a protein specific to the type of microparticle; or (b) administering at least one type of microparticle, each microparticle further comprising at least two types of synthetic nanoliposomes, each type of nanoliposome comprising (i) a cell-free transcription and translation system; and (ii) a plasmid comprising a nucleic acid encoding a protein specific to the type of synthetic nanoliposome.

In some aspects, disclosed herein is a method of treating a disease or medical condition, or of alleviating symptoms thereof, at a focus of interest in a subject in need, said method comprising: administering a synthetic nanoliposome or a microparticle comprising a synthetic nanoliposome to said subject, at or adjacent to a focus of interest, said synthetic nanoliposome comprising: (i) a cell-free transcription and translation system; and (ii) a plasmid comprising a nucleic acid encoding a therapeutic or diagnostic protein; expressing said therapeutic or diagnostic protein; releasing said protein at or adjacent to said focus of interest. In a related aspect, said synthetic nanoliposome further comprises: (iii) a photoactivatable-caged adenosine triphosphate (ATP); and prior to the step of expressing said therapeutic or diagnostic protein, the site of administration is exposed to a light source to photoactivate said photoactivatable-caged ATP.

In some aspects, disclosed herein is method of treating a disease or medical condition, or of alleviating symptoms thereof, at a focus of interest in a subject in need, said method comprising administering synthetic nanoliposomes or microparticles to said subject, at or adjacent to a focus of interest. In some embodiments, the synthetic nanoliposome comprises a photoactivatable-caged ATP and the method further comprises exposing the site of administration to ultraviolet (UV) light or infrared (IR) light. In a related aspect, the disease or medical condition comprises a solid tumor; and the synthetic nanoliposomes or microparticles are administered adjacent to a focus of interest comprising said solid tumor. In a related aspect, the disease or medical condition comprises an autoimmune disease and the synthetic nanoliposomes or microparticles are administered at or adjacent to a focus of interest comprising an autoimmune-targeted or symptomatic focus of said autoimmune disease; the disease or medical condition comprises an allergic reaction, and the synthetic nanoliposomes or microparticles are administered at or adjacent to a focus of interest comprising a reactive focus of said allergic reaction; the disease or medical condition comprises a localized infection or an infectious disease, and the synthetic nanoliposomes or microparticles are administered at or adjacent to a focus of interest comprising a focus of infection or symptoms; the disease or medical condition comprises an injury or a site of chronic damage, and the synthetic nanoliposomes or microparticles are administered at or adjacent to a focus of interest comprising the injury or the site of chronic damage; the disease or medical condition comprises a surgical site, and the synthetic nanoliposomes or microparticles are administered at or adjacent to a focus of interest comprising the surgical site; the disease or medical condition comprises a transplanted organ, tissue, or cells, and the synthetic nanoliposomes or microparticles are administered at or adjacent to a focus of interest comprising a transplant site; the disease or medical condition comprises a blood clot causing or at risk for causing a myocardial infarction, an ischemic stroke, or a pulmonary embolism, and the synthetic nanoliposomes or microparticles are administered at or adjacent to a focus of interest comprising the site of the blood clot. In a related aspect, the administration comprises injection of said nanoliposome or a microparticle. In another related aspect, the injection comprises subcutaneous injection. In a related aspect, the administration comprises administration through a catheter comprising a UV or IR light. In a related aspect, the disease or medical condition comprises a blood clot causing or at risk for causing a myocardial infarction, an ischemic stroke, or a pulmonary embolism, and the synthetic nanoliposomes or microparticles are administered at or adjacent to a focus of interest comprising the site of the blood clot together with angioplasty or another clot removal treatment.

In a related aspect, the method further comprises a step of administering activated T cells to said subject. In a related aspect, administration of T cells is prior to or after exposing the site to UV light. In another related aspect, administration of T cells is prior to or after administering said nanoliposome or said microparticle.

In some aspects, microparticles can be targeted to and be bound to lymphocytes such as T cells, or other blood components, during leukapheresis or other blood cell purification procedure and infused into the patient. Such targeting binding of microparticles to lymphocytes or other cell types after administration to the body or during a leukapheresis procedure or other ex vivo procedure provides the therapeutic protein in association with a cell type to effect its desired function when activated by UV light.

In a related aspect, said method comprises treating a solid tumor, wherein the solid tumor comprises a cancerous, pre-cancerous, or non-cancerous tumor. In a related aspect, the solid tumor comprises a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In a related aspect, methods of treating disclosed herein reduce the size of the solid tumor, eliminates said solid tumor, slows the growth of the solid tumor, or prolongs survival of said subject, or any combination thereof.

In yet another related aspect, methods of treating disclosed herein reduce or eliminate inflammation or another symptom of said autoimmune-targeted or symptomatic focus of said autoimmune disease, prolong survival of said subject, or any combination thereof; reduce or eliminate inflammation or another symptom of allergic reaction at said reactive focus of said allergic reaction, prolong survival of said subject, or any combination thereof; reduce or eliminate infection or symptoms at said focus of infection or symptoms of said localized infection or infectious disease, prolong survival of said subject, or any combination thereof; reduce, eliminate, inhibit, or prevent structural, organ, tissue, or cell damage, inflammation, infection, or another symptom at said site of injury or said site of chronic damage, improve structural, organ, tissue, or cell function at said site of injury or said site of chronic damage, improve mobility of said subject, prolong survival of said subject, or any combination thereof; reduce, eliminate, inhibit, or prevent structural, organ, tissue, or cell damage, inflammation, infection or another symptom at said surgical site, improve structural, organ, tissue, or cell function at said surgical site, improve mobility of said subject, prolong survival of said subject, or any combination thereof; reduce, eliminate, inhibit, or prevent transplanted organ, tissue, or cell damage or rejection, inflammation, infection, or another symptom at said transplant site, improve mobility of said subject, prolong survival of said transplanted organ, tissue, or cell, prolong survival of said subject, or any combination thereof; or reduce or eliminate said blood clot causing or at risk for causing said myocardial infarction, said ischemic stroke, or said pulmonary embolism in said subject, improve function or survival of a heart, brain, or lung organ, tissue or cell in said subject, reduce damage to a heart, brain, or lung organ, tissue, or cell in said subject, prolong survival of a heart, brain, or lung organ, tissue, or cell in said subject, prolong survival of said subject, or any combination thereof.

In a related aspect, the method further comprises (a) administering two or more types of microparticles, each type of microparticle comprising a synthetic nanoliposome comprising (i) a cell-free transcription and translation system; and (ii) a plasmid comprising a nucleic acid encoding a protein specific to the type of microparticle; or (b) administering at least one type of microparticle, each microparticle further comprising at least two types of synthetic nanoliposomes, each type of nanoliposome comprising (i) a cell-free transcription and translation system; and (ii) a plasmid comprising a nucleic acid encoding a protein specific to the type of synthetic nanoliposome.

In some aspects, disclosed herein is a synthetic nanoliposome comprising (a) a cell-free transcription and translation system; (b) a plasmid comprising a nucleic acid encoding a protein, such as a therapeutic or diagnostic protein, polypeptide or peptide; and (c) a photoactivatable-caged ATP, such as a UV-caged ATP. In a related aspect, the therapeutic protein may be a chemokine, a therapeutic antibody or a fragment thereof or antigen-binding protein, a Fc fusion protein, an anticoagulant, an enzyme, a hormone, or a thrombolytic. In a related aspect, the diagnostic protein, polypeptide or peptide may be an antibody or a fragment thereof or antigen-binding protein such as an scFv. In some aspects, disclosed herein is a synthetic nanoliposome comprising (a) a cell-free transcription and translation system; (b) a plasmid comprising a nucleic acid encoding a therapeutic or diagnostic protein, polypeptide or peptide; and (c) a UV-caged ATP. In a related aspect, the size of the nanoliposome comprises about 100-400 nm. In another related aspect, the size of said plasmid comprises about 3000 bp-7000 bp. In a related aspect, the therapeutic protein, polypeptide or peptide may be a chemokine, a therapeutic antibody or a fragment thereof or antigen-binding protein, a Fc fusion protein, an anticoagulant, an enzyme, a hormone, or a thrombolytic. In a related aspect, the diagnostic protein may be an antibody or a fragment thereof or antigen-binding protein such as a scFv.

In some aspects, disclosed herein is a microparticle comprising synthetic nanoliposomes comprising (a) a cell-free transcription and translation system; (b) a plasmid comprising a nucleic acid encoding a therapeutic or diagnostic protein; and (c) a UV-caged ATP. In a related aspect, microparticles comprise about 400 nanoliposomes. In another related aspect, microparticles further comprise superparamagnetic iron oxide nanoparticles. In another related aspect, microparticles further comprise upconversion nanoparticles. In yet another related aspect, microparticles may comprise alginate; or alginate-heparin. In still another related aspect, microparticles comprising alginate or alginate-heparin further comprise a lipid membrane coating. In a related aspect, the lipid membrane comprises POPC.

In some aspects, disclosed herein is method of treating a disease in a subject in need, said method comprising administering microparticles comprising synthetic nanoliposomes comprising a plasmid comprising a nucleic acid encoding a therapeutic protein to said subject, and exposing the site of administration to ultraviolet (UV) or infrared (IR) light. In a related aspect, the administration comprises injection of said nanoliposome or a microparticle. In another related aspect, the injection comprises subcutaneous injection. In some aspects, the disease is cancer, a cardiovascular, neurological, muscular, dermatologic, ophthalmic, or a disease affecting any part of the body accessible to exposure to UV or IR light.

In a related aspect, said methods treating a cancer comprises treating a solid tumor. In a related aspect, a solid tumor comprises a tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In a related aspect, methods of treating disclosed herein reduce the size of the tumor, eliminates said tumor, slows the growth of the tumor, or prolongs survival of said subject, or any combination thereof.

In some aspects, disclosed herein is method of regulating an immune response at the site of a tumor, said method comprising: administering synthetic nanoliposomes or microparticles comprising a plasmid comprising a nucleic acid encoding a therapeutic protein to said subject, adjacent to a solid tumor; and exposing the site of administration to UV light; wherein said regulating the immune response comprises (a) increases proliferation of cytotoxic T cells; (b) increases proliferation of helper T cells; (c) maintains the population of helper T cells at the site of said tumor; (d) activated cytotoxic T cells at the site of said tumor; or any combination thereof. In a related aspect, administration comprises injection of said nanoliposome or a microparticle. In another related aspect, said site comprises an area adjacent to said tumor.

In a related aspect, said tumor comprises a solid tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In some aspects, disclosed herein is method of diagnosing a disease in a subject in need, said method comprising administering synthetic nanoliposomes or microparticles comprising a plasmid comprising a nucleic acid encoding a diagnostic protein to said subject, and exposing the site of administration to UV or IR light. In a related aspect, the administration comprises injection of said nanoliposome or a microparticle. In another related aspect, the injection comprises subcutaneous injection. In some aspects the disease that is diagnosed is cancer, a cardiovascular, neurological, dermatologic, ophthalmic, or a disease affecting a part of the body accessible to exposure to UV or IR light. In a related aspect the diagnostic protein is an antibody or a fragment thereof or antigen-binding protein such as a scFv.

In some aspects, disclosed herein is a method of making a synthetic nanoliposome comprising a cell-free transcription and translation system; a plasmid comprising a nucleic acid encoding a protein; and a photoactivatable-caged adenosine triphosphate (ATP), said method comprising: providing a lipid solution by: combining 1,2-dimyristoyl-sn-glycero-3-phosphocholine (DMPC), 1-palmitoyl-2-oleoyl-phosphatidylcholine (POPC), and cholesterol, to yield a lipid mixture; drying said lipid mixture to form a solid lipid mixture; providing a caged plasmid/cell-free transcription-translation mixture comprising: a cell-free transcription and translation system; and a caged plasmid comprising a nucleic acid encoding a protein; combining said solid lipid mixture and said caged plasmid/cell-free transcription-translation mixture to yield said synthetic nanoliposome. In a related aspect, said caged plasmid/cell-free transcription-translation mixture further comprises a photoactivatable-caged adenosine triphosphate (ATP).

In another related aspect, the combining step comprises: dissolving said solid lipid mixture in a volatile solvent to form a lipid solution; providing a microfluidic device comprising a plurality of channels comprising a center channel and one or more flanking channels; injecting said caged plasmid/cell-free transcription-translation mixture through said center channel of said microfluidic device while simultaneously injecting said lipid solution through one or more channels flanking said center channel of said microfluidic device and optionally simultaneously injecting water or buffer through one or more additional channels in said microfluidic device to yield said synthetic nanoliposome. Alternatively, the combining step comprises: warming said solid lipid mixture to a temperature of about 40 degrees C. to about 90 degrees C.; mixing said caged plasmid/cell-free transcription-translation mixture with said warmed solid lipid mixture; isolating at least one liposome from the mixture, said at least one liposome encapsulating said caged plasmid/cell-free transcription-translation mixture; agitating said at least one liposome in solution; extruding said liposome solution through a porous membrane to provide at least one synthetic nanoliposome comprising said caged plasmid/cell-free transcription-translation mixture to yield said synthetic nanoliposome.

In a related aspect, the method further comprises: providing an alginate solution or an alginate-heparin conjugate solution; mixing said alginate solution or said alginate-heparin conjugate solution with said synthetic nanoliposome to yield an aqueous phase; mixing the aqueous phase with a surfactant in a continuous phase, said continuous phase comprising a surfactant and a non-aqueous solvent to yield a microparticle comprising said nanoliposome; cross-linking said microparticle with an ionic solution; and isolating said microparticle. In some embodiments, said step of agitating said at least one liposome in solution comprises agitating said at least one liposome in deionized water, phosphate buffered saline (PBS), or cell culture medium. In some embodiments, said volatile solvent comprises acetone, chloroform, isopropanol, or methanol. In some embodiments, said caged plasmid is caged with (1-(4,5-dimethoxy-2-nitrophenyl) ethyl ester (DMNPE).

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows a schematic representation of microfluidic inlets. Either water or buffer (e.g., phosphate buffered saline [PBS]) may be used.

FIG. 2 shows a map of the expression plasmid pCellFree_G03_H9 (IL-2-superkine) for the Super2 (synthetic interleukin-2 [IL-2]) cytokine gene, flanked by T7 sites for in vitro transcription. The sequence of pCellFree_G03_H9 with Super2 is SEQ ID NO: 9.

FIG. 3 is a graph depicting the calculated concentration of nanoparticles (NPs) within microfactories based on dynamic light scattering (DLS) measurements of 6.64×10⁸ (6.64×108) NP/mL, which were extracted from a solution containing 1 5×10⁶ (1.5×106) microfactories/mL, thus approximately 443 NP (˜443 NP) per microfactory.

FIGS. 4A-4C shows formation of microparticles and protein production. (FIG. 4A) Schematic representation of microfluidic generation of alginate/alginate-heparin microparticles encapsulating protein producing nanoparticles. These microparticles will then coated with lipid bilayers. (FIG. 4B) Schematic of protein-producing microparticle. (FIG. 4C) Brightfield and fluorescent images of nanoliposome-encapsulated microparticles.

FIG. 5 is a series of graphs depicting T cell viability (% viable T cells) after co-culture with nanofactories or encapsulated nanofactories (microfactories) with interleukin-2 (IL-2) or green fluorescent protein (GFP) plasmids after 4 days of co-culture with CD8+ T cells. Left-to-right: no IL-2, soluble IL-2, no plasmid, free IL-2 plasmid, no plasmid nanofactories, no plasmid microfactories, GFP nanofactories, GFP microfactories, IL-2 nanofactories, IL-2 microfactories.

FIG. 6 is a series of graphs depicting the secretion of granzyme B as measured by mean fluorescence intensity (MFI) as an indicator of activation level of CD8+ T cells after 4 days of co-culture with particles as listed. Intracellular cytokine staining was used to detect presence of granzyme B inside CD8+ T cells. Left-to-right: free IL-2 plasmid, GFP nanofactories, GFP microfactories, IL-2 nanofactories, IL-2 microfactories.

FIG. 7 is a series of graphs depicting the secretion of interferon-gamma (IFN-γ) (μg/mL) on day 4 of co-culture as an indicator of activation level as measured via cytokine ELISA assay. Left-to-right: free IL-2 plasmid, GFP nanofactories, GFP microfactories, IL-2 nanofactories, IL-2 microfactories.

FIGS. 8a-8b are graphs depicting activation and differentiation markers on day 4 and 10 of co-culture including (FIG. 8a) CD62L, and (FIG. 8b) CD25. For each, left-to-right: free nanoparticles, encapsulated nanoparticles (microparticles)+lipid, encapsulated nanoparticles+heparin, encapsulated nanoparticles+lipid+heparin.

FIG. 9 is a logarithmic graph depicting chromium (51Cr, ⁵¹Cr) release assay shows antigen peptide-specific and cytotoxicity of TCR transgenic CD8+ T cells after 4 days of co-culture with particles as listed. Cytotoxic activity was examined at different ratios of 100:1, 30:1, 15:1, 7:1, 3:1, 1.5:1, and 0.75:1 of treated T cells to tumor cells. The data are presented as Mean±SD of 3 independent experiments (open white square=peptide; black closed diamond=free IL-2 plasmid+peptide; closed green square=GFP nanofactories+peptide; closed red circle=cytokine nanofactories+peptide).

FIGS. 10A-10C are a series of graphs depicting tumor growth monitored over time. For the mouse flanks injected with Super2 microfactories, some were UV exposed (“treated”), and some were not (“untreated”). Some mice were not injected with any microfactories but were exposed to the same amount of UV (“Just UV”). Some mice were given (irrelevant) green fluorescent protein (GFP) microfactories. In FIG. 10A, tumor masses were measured upon sacrifice on day 22 for mice injected with microfactories carrying Super2 (IL-2 superkine) (circles) (with [red circles] or without [blue circles] ultraviolet (UV) activation) versus an irrelevant plasmid (GFP) (green squares). Mice with “Just UV” (pale blue triangles) are shown here as a control to demonstrate the effect of UV alone on tumor growth. Each dot is a mouse. FIG. 10B depicts a side-by-side comparison of tumor growth in using PBS without T cells (black), IL-2 injection+Tcells (gray), Just UV+T cells (pale blue), GFP+T cells (no UV treatment) (green), Super2+T cells (no UV treatment) (blue), and Super2+T cells with UV treatment (red). In FIG. 10C, boxes are permuted mean and 95% confidence interval (CI) for Super2+T cells with UV treatment (red circles), Super2+T cells (no UV treatment) (blue circles), GFP+T cells (no UV treatment) (green circles), IL-2 injection+ Tcells (gray circles), Just UV+T cells (pale blue circles), and PBS without T cells (black circles). There are no statistically significant differences between the tumor masses at end-point for the untreated, only-UV treated, and those injected with GFP (irrelevant) plasmid.

FIGS. 11A-11E are a series of graphs depicting in vivo activity of microfactories in a tumor model. All measured by flow cytometry on tumor-infiltrating CD8+ T cells. FIG. 11A shows the levels of Granzyme B percent positivity. FIG. 11B shows the levels of co-expression of CD44 and Granzyme B measured. FIG. 11C shows the proportion of tumor infiltrating lymphocytes (TILs) that showed binding of V-alpha2 (Vα2) antibody (binds to the T cell receptor Vα region of OT-I T cells). FIGS. 11D-11E show the proportion of CD8+ T cells expressing PD-1 (d) and PD-1MFI (e). Each dot is a mouse, boxes show permuted mean and 95% CI. All p values have been adjusted for multiple comparisons.

FIGS. 12A-12F are schematics, photographs, graphs, and scanning electron micrographs (SEM) depicting characterization of nanofactories in vitro. FIG. 12A shows schematics and a photograph demonstrating the microfluidic system used to make monodisperse, synthesis-capable nanoliposomes, which can also encapsulate magnetic and upconverting nanoparticles. Upconversion nanoparticles (UCNPs) are a unique class of optical nanomaterials doped with lanthanide ions featuring a wealth of electronic transitions within the 4f electron shells. These nanoparticles can up-convert two or more lower-energy photons into one high-energy photon (Nature Communications (2018) 9:2415 DOI: 10.1038/s41467-018-04813-5). Practically speaking, upconversion allows infrared (IR) light, which is tissue penetrating, to be converted to blue/UV light, which activates the synthesis. (μF-1-μF-4 and Bulk-1 and Bulk-2 are described in TABLE 2 and TABLE 3.) For any aspects of the invention utilizing ultraviolet (UV) light, by incorporation of UCNPs into the microparticles, IR light can be used for activation. FIGS. 12B-12C present dynamic light scattering (DLS) results used to characterize size of microfluidic-assisted synthesized nanoliposomes at various flow rates as compared to the one that prepared via bulk extrusion. Average hydrodynamic diameter (FIG. 12B) and polydispersity index (PDI) (FIG. 12C) of prepared nanoliposomes for three independent experiments. FIG. 12B is a graph showing the results of a study using dynamic light scattering (DLS) to analyze size of nanoliposomes fabricated by microfluidic flows or bulk synthesis. FIG. 12C is a graph demonstrating that the polydispersity of microfluidically synthesized particles is much smaller (more uniform) than those synthesized by bulk process. FIG. 12D is a graph demonstrating the efficiency of encapsulation of DNA of different sizes as a function of nanoparticle diameter. FIG. 12E is a series of fluorescent images of interleukin-2/green fluorescent protein (IL2-GFP) producing nanoliposomes at 30, 45, and 75 min after UV illumination. Scale bar: 5 microns (μm). FIG. 12F is a graph demonstrating the kinetics of IL2-GFP expression inside nanoliposomes of different sizes after triggering of caged-ATP by UV. Mean+/−SD is shown, n=10.

FIGS. 13A-13D are schematics, photographs, graphs, and scanning electron micrographs (SEM) depicting characterization of microfactories. FIG. 13A shows schematics and a photograph demonstrating the microfluidic generation of alginate-heparin microfactories encapsulating protein producing nanoliposomes (artificial cells). These microfactories were subsequently coated with lipid bilayers. FIG. 13B is a fluorescent image of GFP production in alginate-heparin microfactories 90 min after UV exposure. Lipid membrane around microfactories stained using 1,1-dioctadecyl-3,3,3,3-tetramethylindodicarbocyanine (DiD) fluorescent lipophilic cationic indocarbocyanine dye. DiD has markedly red-shifted fluorescence excitation and emission spectra. FIG. 13C is a graph demonstrating the kinetics of GFP expression inside alginate-heparin microfactories, as depicted in representative fluorescent images. Mean+/−SD is shown, n=10. FIG. 13D is a series of schematics and photographs demonstrating the masking of a portion of a tumor in a mouse and the resulting fluorescence of IL-2-GFP “tattoo” in UV-illuminated region, showing that IL-2 synthesis is local. These results demonstrate the capability of designed microparticles to be delivered and activated in vivo, a mouse skin was “tattooed” with IL2-GFP microparticles, half the area was taped, and the area was exposed to UV light. Within 60 min, GFP production was evident from the exposed area but not from the covered portion, confirming that microparticles can survive in vivo and can produce functional protein after UV activation.

FIGS. 14A-14G are a series of graphs depicting in vitro activity of nanofactories and microfactories. FIG. 14A presents the kinetics of the release of Super2 (synthetic IL-2) from free nanoliposomes as compared to nanoparticles encapsulated within alginate microparticles that are further endowed with a lipid coat or heparin or both after UV triggering of production. The measurements started 2 h post triggering of protein synthesis by UV exposure (n=3). FIG. 14B is a series of graphs depicting the expansion of pre-activated primary mouse T cells cultured in the presence of the indicated synthesis platforms. n=3 independent replicates. Mean shown. Left-to-right: free nanoparticles, encapsulated and lipid-coated nanoparticles, encapsulated+heparin nanoparticles, encapsulated and lipid-coated nanoparticles with heparin. Presented is the expansion of pre-activated primary mouse T cells cultured in presence of various formulation of protein producing platforms. FIG. 14C is a series of graphs depicting T cell viability after co-culture with nanofactories or encapsulated nanofactories, as described. FIGS. 14D-14E each provide a series of flow cytometric analysis graphs demonstrating activation and differentiation markers on day 4 and 10 of co-culture including granzyme B (d), interferon-gamma (IFN-γ) (e). FIG. 14F shows activation markers on day 10 of co-culture with T cells, data representative of 3 independent experiments, using expression of CD25 and CD62L receptors as indicators of T cell activation. Samples: Free nanoliposomes, nanoliposomes encapsulated in alginate-based microparticles coated with lipid membrane, nanoliposomes encapsulated in heparin-modified alginate microparticles without or with lipid coating. FIG. 14G shows the results of chromium (51Cr, ⁵¹Cr) release assays shows antigen peptide-specific and cytotoxicity of TCR transgenic CD8+ T cells after 4 or 10 days of co-culture with particles as listed. Cytotoxic activity was examined at different ratios of 100:1, 30:1, 15:1, 7:1, 3:1, 1.5:1, and 0.75:1 of treated T cells to tumor cells. The data are presented as Mean±SD of 3 independent experiments. Samples: Free nanoliposomes, nanoliposomes encapsulated in alginate-based microparticles coated with lipid membrane, nanoliposomes encapsulated in heparin-modified alginate microparticles without or with lipid coating. Nanofactories: protein (cytokine) producing nanoliposomes.

FIG. 15 presents confocal fluorescent and (inset) scanning electron microscopy image of alginate-RGD scaffold encapsulating GFP-producing NPs, 60 min after UV triggering.

FIGS. 16A-16B describe encapsulated nanofactories for protein particles. FIG. 16A presents a schematic of encapsulated nanofactories comprising protein producing particles. FIG. 16B presents protein producing nanoparticles with or without alginate encapsulating systems co-cultured with CD8 T cells up to 10 days. Cell viability of CD8 T cells after 48 h of culturing with super IL-2 producing microparticles or soluble IL-2. mean±SD (open triangle=free nanoparticles, open circle=encapsulated+lipid membrane, closed triangle=encapsulated+heparin, closed circle=encapsulated heparin+lipid membrane).

FIG. 17 presents protein producing artificial helper T cells can provide on-demand cytokine signaling to control differentiation of cytotoxic T cells (CTLs). Kinetics of expression of main surface markers after priming for 2 days with anti-CD3+anti-CD28. Flow cytometric analysis of CD44 and CD62L co-expression kinetics when cocultured with particles with different formulations at two different time points (left). Percentage of effector cells (CD44+CD62L−) or memory cells (CD44+CD62L+) in each case are plotted for quantitative comparison (right) (open triangle=free nanoparticles, open circle=encapsulated+lipid membrane, closed triangle=encapsulated+heparin, closed circle=encapsulated heparin+lipid membrane).

FIG. 18 presents flow cytometric analysis of CD62L and CD25 expression kinetic over time are represented at day 4 and 10 when activated T cells cocultured with microparticles at different time points. The presented data are expressed as average+/−SD.

FIGS. 19A-19G are schematics and graphs demonstrating how cytokine microfactories eliminate tumors by enhancing T cell activation. FIG. 19A is a series of schematics demonstrating the experimental protocol. FIG. 19B is a graph of results depicting tumor growth over time. FIG. 19C is a graph of results depicting tumor mass at day 22 (note separate vertical axes). On day 22 in tumor T cells: FIG. 19D is a graph depicting CD8 T cells; FIG. 19E is a graph depicting CD8-to-CD4 ratio; FIG. 19F is a graph demonstrating preferential expansion of transferred, antigen-specific T cells; and FIG. 19G is a graph depicting mean fluorescence intensity of granzyme B expression. Each dot is a mouse, box is permuted mean and 95% CI. All p-values have been adjusted for multiple comparisons. (Black circle is PBS control without T cells; red circle is UV-treated side+T cells (uncovered side); blue circle is untreated side+T cells (covered side); and gray circle is IL-2 injection+T cells (control).)

FIG. 20 presents chromium (⁵¹Cr) release assays comparing antigen-specific (OT-I) and non-specific wild-type (WT) cytotoxicity of treated CD8+ T cells with different methods after 12 h of co-culturing T cells and tumor (B16F10-ova) cells. T cells pretreated with various platforms for 4 and 10 days. Cytotoxic activity was examined at seven different ratios (100:1, 30:1, 15:1, 7:1, 3:1, 1.5:1, and 0.75:1) of treated T cells to tumor cells. The data are presented as average±SD of three independent experiments. The presented data are expressed as average±SD. At day 4 and day 10 of the experiments, antigen-specific and non-specific T cells were significantly different (p<0.001).

FIG. 21 presents graphs representing individual follow-up of tumor size for mice with different treatments (n=5).

FIG. 22 presents representative quantitative analysis of effector (GranzymeB⁺, CD44^{high}) T cells (gated on CD8+T cells) in tumors analyzed by flow cytometry.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION

In the following detailed description, numerous specific details are set forth in order to provide a thorough understanding of nanoliposomes, microparticles, and the uses thereof for treating cancer or other tumors. However, it will be understood by those skilled in the art that the production of these nanoliposomes and microparticles and uses thereof may be practiced without these specific details. In other instances, well-known methods, procedures, and components have not been described in detail so as not to obscure their description.

In some aspects, disclosed herein is a synthetic nanoliposome comprising (a) a cell-free transcription and translation system; and (b) a plasmid comprising a nucleic acid encoding a protein. In a related aspect, the nanoliposome further comprises: (c) a photoactivatable-caged adenosine triphosphate (ATP). In a related aspect, the photoactivatable-caged ATP comprises an ultraviolet (UV)-caged ATP or an infrared (IR)-caged ATP. In a related aspect, the size of the nanoliposome comprises about 100-400 nm. In another related aspect, the size of said plasmid comprises about 3000 bp-7000 bp. In another related aspect said plasmid comprises an expression plasmid. In another related aspect, the protein comprises a therapeutic or diagnostic protein. In another related aspect, the protein comprises a cytokine, a chemokine, a therapeutic or diagnostic antibody or fragment thereof, an antigen-binding protein, a Fc fusion protein, an anticoagulant, an enzyme, a hormone, or a thrombolytic. In another related aspect, the cytokine comprises an interleukin. In another related aspect, the interleukin comprises an IL-2, an IL-4, an IL-10, an IL-12, or an IL-15. In yet another related aspect, a cytokine may include a human cytokine. In still another related aspect, an IL-2 cytokine comprises an IL-2 superkine. In another related aspect, the IL-2 superkine is encoded by the nucleotide sequence as set forth in SEQ ID NO: 7. In another related aspect, the plasmid comprises a pCellFree_G03_H9 plasmid expressing the IL-2 superkine. In yet another related aspect, the plasmid has the nucleotide sequence as set forth in SEQ ID NO: 9.

In some aspects, disclosed herein is a microparticle comprising at least one synthetic nanoliposome, the at least one nanoliposome comprising (a) a cell-free transcription and translation system; and (b) a plasmid comprising a nucleic acid encoding a protein. In a related aspect, the synthetic nanoliposome further comprises: (c) a photoactivatable-caged adenosine triphosphate (ATP). In a related aspect, the photoactivatable-caged ATP comprises an ultraviolet (UV)-caged ATP or an infrared (IR)-caged ATP. In a related aspect, microparticles comprise about 400 nanoliposomes. In another related aspect, microparticles further comprise superparamagnetic iron oxide nanoparticles (SPION, SION). In another related aspect, microparticles further comprise upconversion nanoparticles (UCNPs). In yet another related aspect, microparticles may comprise alginate; or alginate-heparin. In still another related aspect, microparticles comprising alginate or alginate-heparin further comprise a lipid membrane coating. In a related aspect, the lipid membrane comprises 1-palmitoyl-2-oleoyl-sn-glycero-3-phosphocholine (POPC; IUPAC [(2R)-3-hexadecanoyloxy-2-[(Z)-octadec-9-enoyl]oxypropyl]2-(trimethylazaniumyl)ethyl phosphate). In a related aspect, the microparticle further comprises at least two types of synthetic nanoliposomes, each type of nanoliposome comprising (a) a cell-free transcription and translation system; (b) a plasmid comprising a nucleic acid encoding a protein specific to the type of synthetic nanoliposome; and (c) a photoactivatable-caged ATP.

In some aspects, disclosed herein is a method of regulating an immune response at a focus of interest in a subject in need, said method comprising: (a) administering a synthetic nanoliposome or a microparticle comprising a synthetic nanoliposome to said subject, at or adjacent to said focus of interest, said synthetic nanoliposome comprising: a cell-free transcription and translation system; a plasmid comprising a nucleic acid encoding a protein; (c) expressing said therapeutic or diagnostic protein; and (d) releasing said protein at or adjacent to said focus of interest, said regulating the immune response comprising: increasing proliferation of cytotoxic T cells; increasing proliferation of helper T cells; maintaining the population of helper T cells at the site of said focus of interest; activating cytotoxic T cells at the site of said focus of interest; or any combination thereof. In a related aspect, the synthetic nanoliposome further comprises a photoactivatable-caged adenosine triphosphate (ATP) and prior to the step of expressing said therapeutic or diagnostic protein, said site of administration is exposed to a light source to photoactivate said photoactivatable-caged ATP.

In a related aspect, the photoactivatable-caged ATP comprising an ultraviolet (UV)-caged ATP or an infrared (IR)-caged ATP, and said administering comprising administering of said synthetic nanoliposome or said microparticle through a catheter comprising a UV or IR light. In another aspect, said administration comprises injection of said synthetic nanoliposome or said microparticle. In still another aspect, the injection comprises subcutaneous injection.

In another related aspect, the method further comprising a step of administering activated T cells to said subject. In another aspect, administering of said activated T cells is concomitant with administering said synthetic nanoliposome or said microparticle or is prior to or after administering said synthetic nanoliposome or said microparticle. In yet another aspect, the photoactivatable-caged ATP comprising an ultraviolet (UV)-caged ATP or an infrared (IR)-caged ATP, and said administering of said activated T cells is prior to or after exposing the site to UV or IR light.

In a related aspect, said focus of interest comprises a solid tumor. In another related aspect, said focus of interest comprises an autoimmune-targeted or symptomatic focus of an autoimmune disease; a reactive focus of an allergic reaction or hypersensitivity reaction, a focus of infection or symptoms of a localized infection or infectious disease; an injury or a site of chronic damage; a surgical site; a site of a transplanted organ, tissue, or cell; or a site of a blood clot causing or at risk for causing a myocardial infarction, ischemic stroke, or pulmonary embolism. In a related aspect, administration comprises injection of said nanoliposome or a microparticle. In a related aspect, administration comprises administration of said nanoliposome or microparticle through a catheter comprising a UV or

IR light. In a related aspect, where said focus of interest comprises a blood clot causing or at risk for causing a myocardial infarction, an ischemic stroke, or a pulmonary embolism, the administration is at or adjacent to the site of the blood clot together with angioplasty or another clot removal treatment. In another related aspect, said site comprises an area adjacent to said tumor. In another related aspect, said site comprises an area at or adjacent to said autoimmune-targeted or symptomatic focus of an autoimmune disease; said reactive focus of an allergic reaction or hypersensitivity reaction; said focus of infection or symptoms or a localized infection or an infectious disease; said injury or site of chronic damage; said surgical site; said site of a transplanted organ, tissue, or cell; or said site of a blood clot causing or at risk for causing a myocardial infarction, ischemic stroke, or pulmonary embolism.

In a further related aspect, said method further comprises a step of administering activated T cells to said subject. In a related aspect, the administration of said activated T cells is prior to or after administering said nanoliposome or said microparticle. In another related aspect, administration of said activated T cells is prior to or after exposing the site to UV light.

In some aspects, microparticles can be targeted to and be bound to lymphocytes such as T cells, or other blood components, during leukapheresis or other blood cell purification procedure and infused into the patient. Such targeting binding of microparticles to lymphocytes or other cell types after administration to the body or during a leukapheresis procedure or other ex vivo procedure provides the therapeutic protein in association with a cell type to effect its desired regulation of an immune response when activated by UV light.

In a related aspect, said tumor comprises a solid tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In yet another aspect, the method further comprises (a) administering two or more types of microparticles, each type of microparticle comprising a synthetic nanoliposome comprising (i) a cell-free transcription and translation system; (ii) a plasmid comprising a nucleic acid encoding a protein specific to the type of microparticle; and (iii) a photoactivatable-caged adenosine triphosphate (ATP); or (b) administering at least one type of microparticle, each microparticle further comprising at least two types of synthetic nanoliposomes, each type of nanoliposome comprising (i) a cell-free transcription and translation system; (ii) a plasmid comprising a nucleic acid encoding a protein specific to the type of synthetic nanoliposome; and (iii) a photoactivatable-caged adenosine triphosphate (ATP).

In some aspects, disclosed herein is a method of treating a disease or medical condition, or of alleviating symptoms thereof, at a focus of interest in a subject in need, said method comprising: administering a synthetic nanoliposome or a microparticle comprising a synthetic nanoliposome to said subject, at or adjacent to a focus of interest, said synthetic nanoliposome comprising: (i) a cell-free transcription and translation system; and (ii) a plasmid comprising a nucleic acid encoding a therapeutic or diagnostic protein; expressing said therapeutic or diagnostic protein; releasing said protein at or adjacent to said focus of interest. In a related aspect, said synthetic nanoliposome further comprises: (iii) a photoactivatable-caged adenosine triphosphate (ATP); and prior to the step of expressing said therapeutic or diagnostic protein, the site of administration is exposed to a light source to photoactivate said photoactivatable-caged ATP.

In some aspects, disclosed herein is method of treating a disease or medical condition, or of alleviating symptoms thereof, at a focus of interest in a subject in need, said method comprising administering synthetic nanoliposomes or microparticles to said subject, at or adjacent to a focus of interest; and exposing the site of administration to ultraviolet (UV) light or infrared (IR) light. In a related aspect, the disease or medical condition comprises a solid tumor; and the synthetic nanoliposomes or microparticles are administered adjacent to a focus of interest comprising said solid tumor. In a related aspect, the disease or medical condition comprises an autoimmune disease and the synthetic nanoliposomes or microparticles are administered at or adjacent to a focus of interest comprising an autoimmune-targeted or symptomatic focus of said autoimmune disease; the disease or medical condition comprises an allergic reaction, and the synthetic nanoliposomes or microparticles are administered at or adjacent to a focus of interest comprising a reactive focus of said allergic reaction; the disease or medical condition comprises a localized infection or an infectious disease, and the synthetic nanoliposomes or microparticles are administered at or adjacent to a focus of interest comprising a focus of infection or symptoms; the disease or medical condition comprises an injury or a site of chronic damage, and the synthetic nanoliposomes or microparticles are administered at or adjacent to a focus of interest comprising the injury or the site of chronic damage; the disease or medical condition comprises a surgical site, and the synthetic nanoliposomes or microparticles are administered at or adjacent to a focus of interest comprising the surgical site; the disease or medical condition comprises a transplanted organ, tissue, or cells, and the synthetic nanoliposomes or microparticles are administered at or adjacent to a focus of interest comprising a transplant site; the disease or medical condition comprises a blood clot causing or at risk for causing a myocardial infarction, an ischemic stroke, or a pulmonary embolism, and the synthetic nanoliposomes or microparticles are administered at or adjacent to a focus of interest comprising the site of the blood clot. In a related aspect, the administration comprises injection of said nanoliposome or a microparticle. In another related aspect, the injection comprises subcutaneous injection. In a related aspect, the administration comprises administration through a catheter comprising a UV or IR light. In a related aspect, the disease or medical condition comprises a blood clot causing or at risk for causing a myocardial infarction, an ischemic stroke, or a pulmonary embolism, and the synthetic nanoliposomes or microparticles are administered at or adjacent to a focus of interest comprising the site of the blood clot together with angioplasty or another clot removal treatment.

In a related aspect, the method further comprises a step of administering activated T cells to said subject. In a related aspect, administration of T cells is prior to or after exposing the site to UV light. In another related aspect, administration of T cells is prior to or after administering said nanoliposome or said microparticle.

In some aspects, microparticles can be targeted to and be bound to lymphocytes such as T cells, or other blood components, during leukapheresis or other blood cell purification procedure and infused into the patient. Such targeting binding of microparticles to lymphocytes or other cell types after administration to the body or during a leukapheresis procedure or other ex vivo procedure provides the therapeutic protein in association with a cell type to effect its desired function when activated by UV light.

In a related aspect, said method comprises treating a solid tumor, wherein the solid tumor comprises a cancerous, pre-cancerous, or non-cancerous tumor. In a related aspect, the solid tumor comprises a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In a related aspect, methods of treating disclosed herein reduce the size of the solid tumor, eliminates said solid tumor, slows the growth of the solid tumor, or prolongs survival of said subject, or any combination thereof.

In yet another related aspect, methods of treating disclosed herein reduce or eliminate inflammation or another symptom of said autoimmune-targeted or symptomatic focus of said autoimmune disease, prolong survival of said subject, or any combination thereof; reduce or eliminate inflammation or another symptom of allergic reaction at said reactive focus of said allergic reaction, prolong survival of said subject, or any combination thereof; reduce or eliminate infection or symptoms at said focus of infection or symptoms of said localized infection or infectious disease, prolong survival of said subject, or any combination thereof; reduce, eliminate, inhibit, or prevent structural, organ, tissue, or cell damage, inflammation, infection, or another symptom at said site of injury or said site of chronic damage, improve structural, organ, tissue, or cell function at said site of injury or said site of chronic damage, improve mobility of said subject, prolong survival of said subject, or any combination thereof; reduce, eliminate, inhibit, or prevent structural, organ, tissue, or cell damage, inflammation, infection or another symptom at said surgical site, improve structural, organ, tissue, or cell function at said surgical site, improve mobility of said subject, prolong survival of said subject, or any combination thereof; reduce, eliminate, inhibit, or prevent transplanted organ, tissue, or cell damage or rejection, inflammation, infection, or another symptom at said transplant site, improve mobility of said subject, prolong survival of said transplanted organ, tissue, or cell, prolong survival of said subject, or any combination thereof; or reduce or eliminate said blood clot causing or at risk for causing said myocardial infarction, said ischemic stroke, or said pulmonary embolism in said subject, improve function or survival of a heart, brain, or lung organ, tissue or cell in said subject, reduce damage to a heart, brain, or lung organ, tissue, or cell in said subject, prolong survival of a heart, brain, or lung organ, tissue, or cell in said subject, prolong survival of said subject, or any combination thereof.

In a related aspect, the method further comprises (a) administering two or more types of microparticles, each type of microparticle comprising a synthetic nanoliposome comprising (i) a cell-free transcription and translation system; (ii) a plasmid comprising a nucleic acid encoding a protein specific to the type of microparticle; and (iii) a photoactivatable-caged ATP; or (b) administering at least one type of microparticle, each microparticle further comprising at least two types of synthetic nanoliposomes, each type of nanoliposome comprising (i) a cell-free transcription and translation system; (ii) a plasmid comprising a nucleic acid encoding a protein specific to the type of synthetic nanoliposome; and (iii) a photoactivatable-caged ATP.

In some aspects, disclosed herein is a synthetic nanoliposome comprising (a) a cell-free transcription and translation system; (b) a plasmid comprising a nucleic acid encoding a protein, such as a therapeutic or diagnostic protein, polypeptide or peptide; and (c) a photoactivatable-caged ATP, such as a UV-caged ATP. In a related aspect, the therapeutic protein may be a chemokine, a therapeutic antibody or a fragment thereof or antigen-binding protein, a Fc fusion protein, an anticoagulant, an enzyme, a hormone, or a thrombolytic. In a related aspect, the diagnostic protein, polypeptide or peptide may be an antibody or a fragment thereof or antigen-binding protein such as an scFv. In some aspects, disclosed herein is a synthetic nanoliposome comprising (a) a cell-free transcription and translation system; (b) a plasmid comprising a nucleic acid encoding a therapeutic or diagnostic protein, polypeptide or peptide; and (c) a UV-caged ATP. In a related aspect, the size of the nanoliposome comprises about 100-400 nm. In another related aspect, the size of said plasmid comprises about 3000 bp-7000 bp. In a related aspect, the therapeutic protein, polypeptide or peptide may be a chemokine, a therapeutic antibody or a fragment thereof or antigen-binding protein, a Fc fusion protein, an anticoagulant, an enzyme, a hormone, or a thrombolytic. In a related aspect, the diagnostic protein may be an antibody or a fragment thereof or antigen-binding protein such as a scFv.

In some aspects, disclosed herein is a microparticle comprising synthetic nanoliposomes comprising (a) a cell-free transcription and translation system; (b) a plasmid comprising a nucleic acid encoding a therapeutic or diagnostic protein; and (c) a UV-caged ATP. In a related aspect, microparticles comprise about 400 nanoliposomes. In another related aspect, microparticles further comprise superparamagnetic iron oxide nanoparticles. In another related aspect, microparticles further comprise upconversion nanoparticles. In yet another related aspect, microparticles may comprise alginate; or alginate-heparin. In still another related aspect, microparticles comprising alginate or alginate-heparin further comprise a lipid membrane coating. In a related aspect, the lipid membrane comprises POPC.

In some aspects, disclosed herein is method of treating a disease in a subject in need, said method comprising administering microparticles comprising synthetic nanoliposomes comprising a plasmid comprising a nucleic acid encoding a therapeutic protein to said subject, and exposing the site of administration to ultraviolet (UV) or infrared (IR) light. In a related aspect, the administration comprises injection of said nanoliposome or a microparticle. In another related aspect, the injection comprises subcutaneous injection. In some aspects, the disease is cancer, a cardiovascular, neurological, muscular, dermatologic, ophthalmic, or a disease affecting any part of the body accessible to exposure to UV or IR light.

In a related aspect, said methods treating a cancer comprises treating a solid tumor. In a related aspect, a solid tumor comprises a tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In a related aspect, methods of treating disclosed herein reduce the size of the tumor, eliminates said tumor, slows the growth of the tumor, or prolongs survival of said subject, or any combination thereof.

In some aspects, disclosed herein is method of regulating an immune response at the site of a tumor, said method comprising: administering synthetic nanoliposomes or microparticles comprising a plasmid comprising a nucleic acid encoding a therapeutic protein to said subject, adjacent to a solid tumor; and exposing the site of administration to UV light; wherein said regulating the immune response comprises (a) increases proliferation of cytotoxic T cells; (b) increases proliferation of helper T cells; (c) maintains the population of helper T cells at the site of said tumor; (d) activated cytotoxic T cells at the site of said tumor; or any combination thereof. In a related aspect, administration comprises injection of said nanoliposome or a microparticle. In another related aspect, said site comprises an area adjacent to said tumor.

In a related aspect, said tumor comprises a solid tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

In some aspects, disclosed herein is method of diagnosing a disease in a subject in need, said method comprising administering synthetic nanoliposomes or microparticles comprising a plasmid comprising a nucleic acid encoding a diagnostic protein to said subject, and exposing the site of administration to UV or IR light. In a related aspect, the administration comprises injection of said nanoliposome or a microparticle. In another related aspect, the injection comprises subcutaneous injection. In some aspects the disease that is diagnosed is cancer, a cardiovascular, neurological, dermatologic, ophthalmic, or a disease affecting a part of the body accessible to exposure to UV or IR light. In a related aspect the diagnostic protein is an antibody or a fragment thereof or antigen-binding protein such as a scFv.

In some embodiments, described herein are synthetic nanoliposomes. These synthetic nanoliposomes may be consider as “artificial cells” or “liposomal nanofactories” or “nanofactories”, able to produce and provide biological molecules to a microenvironment within which they are located. The production of biomolecules may be regulatable, providing targeted therapeutic biological molecule(s) or biological molecule(s) that in turn regulates a downstream therapeutic target. This regulatable production may in certain embodiments, reduce or eliminate systemic toxicity.

In some embodiments, a collection of synthetic nanoliposomes may be brought together forming microparticles. These microparticles may be considered to be a collection of “artificial cells”/liposomal nanofactories” that produce and provide an increased amount of a biomolecule, while retaining the regulatable aspects of the production and the localized distribution. These “microparticle factories” may be targeted to a site of need by incorporating targeting molecules into an encapsulation coating. Additionally, activation biomolecules may in certain embodiments be incorporated into an encapsulation coating, ensuring an activated target for a biomolecule produced by the liposomal nanofactories. In some embodiments, microparticles may be further coated with a lipid membrane, which may enhance the biophysical properties of the microparticles.

In some embodiments, described herein are uses of these “liposomal nanofactories” (“nanofactories”) or “microparticle factories” (“microfactories”) for treating cancer or other tumors. Use of these “factories” in a therapeutic cancer or tumor treatment may in certain embodiments, prove advantageous as they may provide a regulatable expression system of a needed or advantageous biomolecule, within a localized treatment area that may further be targeted to T cells, which in turn could promote clearance of the cancer or tumor.

In some embodiments, described herein are uses of these “liposomal nanofactories” or “microparticle factories” for treating a focus of interest of an autoimmune disease or an allergic reaction, a localized site of an infection or infectious disease, a localized site of an injury or other damage, a transplant or other surgical site, a blood clot, or a symptom thereof, or a combination thereof. Use of these “factories” in the treatment of a focus of interest of an autoimmune disease or an allergic reaction, a localized site of an infection or infectious disease, a localized site of an injury or other damage, a transplant or other surgical site, a blood clot, or a symptom thereof, or a combination thereof, may in certain embodiments, prove advantageous as they may provide a regulatable expression system of a needed or advantageous biomolecule, within a localized treatment area that may further be targeted to T cells, which could promote clearance of or alleviate localized symptoms of the autoimmune disease, allergic reaction, infection or infectious disease, or blood clot, or could facilitate healing and/or prevent infection or rejection of a localized site of an injury or other damage, a transplant or other surgical site, or could alleviate localized symptoms thereof.

### Nanoliposomes

In some embodiments, disclosed herein is a synthetic nanoliposome comprising: (a) a cell-free transcription and translation system; (b) a plasmid comprising a nucleic acid encoding a cytokine; and (c) a UV-caged ATP.

Nanoliposomes are bilayer lipid vesicles. Synthesis of nanoliposomes is well known in the art, for example, but not limited to, that described in Hasani-Sadrabadi et al., (2016) Advanced Materials 28(21): 4134-4141.

Cell-free transcription and translation systems equip these nanoliposomes to transcribe and translate biomolecules encoded by nucleic acid sequences also present within the nanoliposome. In some embodiments, a cell-free transcription and translation system is used for in vivo protein expression. In some embodiments, a cell-free translation system is used for protein expression of an mRNA molecule. In some embodiments, a cell-free transcription and translation system is used to express a single protein. In some embodiments, a cell-free transcription and translation system is used to express multiple proteins.

Cell-free transcription and translation systems are well known in the art. In some embodiments, a cell-free transcription and translation system comprises an E. coli extract.

In some embodiments, a cell-free transcription and translation system enables direct access and control of the translation environment, which is advantageous for a number of applications including optimization of protein production. In some embodiments, a cell-free transcription and translation system provides a controlled expression of a protein or proteins expressed. In some embodiments, a cell-free transcription and translation system provides sustained release of a protein or proteins expressed. In some embodiments, a cell-free transcription and translation system provides controlled expression and sustained release of a protein or proteins expressed. In some embodiments, a cell-free transcription and translation system provides a controlled expression of a protein or proteins encoded by a nucleic acid.

In some embodiments, regulation of a cell-free transcription and translation system may be at the level of transcription, wherein a nucleic acid to be transcribed comprises regulatable elements, operably linked to an open reading frame(s) encoding a polypeptide.

The term “operably linked” encompasses components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein encompasses polynucleotide sequences that can affect expression or processing of coding sequences to which they are ligated or operably linked. The nature of such control sequences may depend upon the elements included in the cell-free transcription and translation system. In particular embodiments, transcription control sequences may include a promoter, ribosomal binding site, and transcription termination sequence. In some embodiments, transcription control sequences may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as used herein encompasses single-stranded or double-stranded nucleic acid polymers. In certain embodiments, the nucleotides comprising the polynucleotide can be ribonucleotides or deoxyribonucleotides or a modified form of either type of nucleotide. The term “polynucleotide” specifically includes single and double stranded forms of DNA.

In some embodiments, a synthetic nanoliposome disclosed herein comprises a plasmid comprising a nucleic acid encoding a cytokine. In some embodiments, the plasmid comprises a nucleic acid comprising control sequences operably linked to the nucleic acid sequence encoding the cytokine. In some embodiments, the plasmid comprises an expression vector.

In some embodiments, the expression vector may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter, or any promotor sequence known in the art Enhancer sequences may be selected to enhance the transcription of the polynucleotide.

Transcription and translation of an encoded cytokine by the cell-free machinery are ATP-dependent processes. Therefore, availability of ATP, may in certain embodiments, regulate expression of a cytokine encoded by a plasmid vector. Caged ATP molecules may be used to regulate ATP release rapidly at sites of biological interest, at a desired time.

In some aspects, disclosed herein is a nanoliposome comprising a cell-free transcription and translation system, a plasmid comprising a nucleic acid encoding a cytokine, and a photoactivatable ATP. In some embodiments, the photoactivatable ATP comprises a UV caged ATP. In certain embodiments, a nanoliposome comprising a cell-free transcription and translation system, a plasmid comprising a nucleic acid encoding a cytokine, and a UV caged ATP comprises a regulatable cytokine expression system. Upon “uncaging” the ATP (UV caged ATP) by light activation, a pulse of ATP is released and is available to initiate the processes of transcription and translation of the encoded cytokine. Prior to light activation, the ATP molecule remains protected and unavailable.

A skilled artisan would appreciate and know how to select a caged ATP molecule, wherein they are well known in the art. In some embodiments, a caged ATP molecule may encompass a photoactivatable ATP molecule, for example but not limited to DMNPE-caged ATP (Adenosine 5′-Triphosphate, P3-(1-(4,5-Dimethoxy-2-Nitrophenyl)ethyl) Ester, Disodium Salt; THERMO FISHER SCIENTIFIC™ Cat No. A1049) or NPE-caged ATP (Adenosine 5′-Triphosphate, P3-(1-(2-Nitrophenyl)Ethyl) Ester, Disodium Salt; THERMO FISHER SCIENTIFIC™ Cat No. A1048; Jena Science, Germany Cat. No. NU-301S).

In some embodiments, immune cells, for example T cell, are generated and expanded by the presence of cytokines in vivo. In some embodiments, cytokines that affect generation and maintenance to T-helper cells in vivo comprise IL-2, IL-12, and IL-15. In some embodiments, T regulatory (Treg) cells are generated from naïve T cells by cytokine induction in vivo. In some embodiments, TGF-β and/or IL-2 play a role in differentiating naïve T cell to become Treg cells.

“Cytokines” are a category of small proteins (˜5-20 kDa) critical to cell signaling. Cytokines are peptides and usually are unable to cross the lipid bilayer of cells to enter the cytoplasm. Among other functions, cytokines may be involved in autocrine, paracrine and endocrine signaling as immunomodulating agents. Cytokines may be pro-inflammatory or anti-inflammatory. Cytokines include, but are not limited to, chemokines (cytokines with chemotactic activities), interferons, interleukins (ILs; cytokines made by one leukocyte and acting on one or more other leukocytes), lymphokines (produced by lymphocytes), monokines (produced by monocytes), and tumor necrosis factors. Cells producing cytokines include, but are not limited to, immune cells (e.g., macrophages, B lymphocytes, T lymphocytes and mast cells), as well as endothelial cells, fibroblasts, and various stromal cells. A particular cytokine may be produced by more than one cell type.

A skilled artisan would appreciate that the term “cytokine” may encompass cytokines beneficial to enhancing an immune response targeted against a cancer or a pre-cancerous or non-cancerous tumor or lesion. A skilled artisan would also appreciate that the term “cytokine” may encompass cytokines beneficial to enhancing an immune response against a disease or inflammation (e.g., resulting from surgery, an injury, or damage from an autoimmune response) or that the term “cytokine” may encompass cytokines beneficial to reducing an abnormal autoimmune response.

In some embodiments, a cytokine encoded by the nucleic acid expands and maintains T-helper cells (helper T cells). In some embodiments, a cytokine encoded by the nucleic acid expands T-helper cells. In some embodiments, a cytokine encoded by the nucleic acid maintains T-helper cells. In some embodiments, a cytokine encoded by the nucleic acid expands cytotoxic T cells (CTLs). In some embodiments, a cytokine encoded by the nucleic acid activates cytotoxic T cells. In some embodiments, a cytokine encoded by the nucleic acid expands and activates cytotoxic T cells. In some embodiments, a cytokine encoded by the nucleic acid increases proliferation of a T-helper cell population. In some embodiments, a cytokine encoded by the nucleic acid increases proliferation of a cytotoxic T cell population.

In some embodiments, the encoded cytokine comprises an interleukin (IL). A skilled artisan would appreciate that interleukins comprise a large family of molecules, including, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17A, IL-17F, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36.

In some embodiments, the encoded interleukin comprises an IL-2, IL-12, or an IL-15, or any combination thereof. In some embodiments, the encoded cytokine comprises an IL-2. In some embodiments, the encoded cytokine comprises an IL-12. In some embodiments, the encoded cytokine comprises an IL-15.

In some embodiments, the cytokine itself may be a fusion protein with a portion of the receptor, as a way of facilitating the delivery.

In some embodiments, the plasmid encodes any additional cytokine or polypeptide or peptide.

In some embodiments, the IL-2 cytokine comprises an IL-2 superkine (super IL-2 cytokine; Super2). IL-2 is a 133 amino acid glycoprotein with one intramolecular disulfide bond and variable glycosylation.

“IL-2 superkine” (Fc) or “Super2” is an artificial variant of IL-2 containing mutations at positions L80F/R81D/L85V/I86V/I92F. These mutations are located in the molecule's core that acts to stabilize the structure and to give it a receptor-binding conformation mimicking native IL-2 bound to CD25. These mutations effectively eliminate the functional requirement of IL-2 for CD25 expression and elicit proliferation of T cells. Compared to IL-2, the IL-2 superkine induces superior expansion of cytotoxic T cells, leading to improved antitumor responses in vivo, and elicits proportionally less toxicity by lowering the expansion of T regulatory cells and reducing pulmonary edema.

A “T cell” is characterized and distinguished by the T cell receptor (TCR) on the surface. A T cell is a type of lymphocyte that arises from a precursor cell in the bone marrow before migrating to the thymus, where it differentiates into one of several kinds of T cells. Differentiation continues after a T cell has left the thymus. A “cytotoxic T cell” (CTL) is a CD8+ T cell able to kill, e.g., virus-infected cells or cancer cells. A “T helper cell” is a CD4+ T cell that interacts directly with other immune cells (e.g., regulatory B cells) and indirectly with other cells to recognize foreign cells to be killed. “Regulatory T cells” (T regulatory cells; Treg), also known as “suppressor T cells,” enable tolerance and prevent immune cells from inappropriately mounting an immune response against “self,” but may be co-opted by cancer or other cells. In autoimmune disease, “self-reactive T cells” mount an immune response against “self” that damages healthy, normal cells.

A nanoliposome comprising a cell-free transcription and translation system, a plasmid comprising a nucleic acid encoding a cytokine, and a UV caged ATP, in certain embodiments, provides a regulatable cytokine expression system, wherein regulatable expression of the cytokine by the nanoliposome provides a regulatable source of a cytokine within a localize region.

As used herein, the terms “nanoparticle”, “nanoliposome”, “liposomal nanoparticle”, “liposomal NP”, “NP”, “liposomal nanofactories”, and “nanofactories” may be used interchangeably, having all the same limitations and meanings, wherein a “liposomal nanofactory” encompasses a synthetic nanoliposome comprising a cell-free transcription and translation system, a plasmid comprising a nucleic acid encoding a cytokine, and optionally, a photoactivatable-caged ATP. Alternatively, a “nanoparticle” may be used to indicate an untreated nanoparticle.

The size of a nanoliposome may in certain embodiments, effect the quantity or size of plasmids that may be included within the nanoliposome. In some embodiments, the size of a nanoliposome is about 50-500 nm. In some embodiments, the size of a nanoliposome is about 100-500 nm. In some embodiments, the size of a nanoliposome is about 100-400 nm. In some embodiments, the size of a nanoliposome is about 100-300 nm. In some embodiments, the size of a nanoliposome is about 100-200 nm. In some embodiments, the size of a nanoliposome is about 200-400 nm. In some embodiments, the size of a nanoliposome is about 200-300 nm.

In some embodiments, the size of a nanoliposome is about 50 nm. In some embodiments, the size of a nanoliposome is about 100 nm. In some embodiments, the size of a nanoliposome is about 150 nm. In some embodiments, the size of a nanoliposome is about 200 nm. In some embodiments, the size of a nanoliposome is about 250 nm. In some embodiments, the size of a nanoliposome is about 300 nm. In some embodiments, the size of a nanoliposome is about 350 nm. In some embodiments, the size of a nanoliposome is about 400 nm. In some embodiments, the size of a nanoliposome is about 450 nm. In some embodiments, the size of a nanoliposome is about 500 nm.

In some embodiments, the size of a plasmid may affect the number and efficacy of transcription and translation. In some embodiments, the size of a plasmid comprises about 2000 bp-9000 bp. In some embodiments, the size of a plasmid comprises about 3000 bp-8000 bp. In some embodiments, the size of a plasmid comprises about 3000 bp-7000 bp. In some embodiments, the size of a plasmid comprises about 3000 bp-6000 bp. In some embodiments, the size of a plasmid comprises about 3000 bp-5000 bp.

In some embodiments, the size of a plasmid comprises about 2000 bp, 3000 bp, 4000 bp, 5000 bp, 6000 bp, 7000 bp, 8000 bp, or 9000 bp. Plasmids comprising expression vectors are well known in the art, wherein a skilled artisan would be able to design and produce a plasmid able to express a cytokine and be incorporated within a nanoliposome.

### Microparticles

In some embodiments, larger structures composed of many hundreds of nanoliposome factories, as described above, may be produced. In certain embodiments, disclosed herein are microparticle comprising one or more synthetic nanoliposomes comprising: (a) a cell-free transcription and translation system; (b) a plasmid comprising a nucleic acid encoding a cytokine; and (c) a UV-caged ATP. The qualities and technical characteristics of said nanoliposome has been described above in detail. The cytokine comprised in the nanoliposome is also described above. In some embodiments, said cytokine comprises an interleukin (IL). In some embodiments, said cytokine comprises an IL-2, an IL-12, or an IL-15. In some embodiments, said IL-2 comprises an IL-2 superkine.

In some embodiments, the microparticle disclosed here, comprises between about 100-1000 nanoliposomes. In some embodiments, the microparticle disclosed here, comprises between about 500-1000 nanoliposomes. In some embodiments, the microparticle disclosed here, comprises between about 100-500 nanoliposomes. In some embodiments, the microparticle disclosed here, comprise between about 200-900 nanoliposomes. In some embodiments, the microparticle disclosed here, comprises between about 300-800 nanoliposomes. In some embodiments, the microparticle disclosed here, comprises between about 400-500 nanoliposomes.

In some embodiments, a microparticle comprises about 100, 200, 300, 400, 500, 600, 700, 800, 900, or 1,000 nanoliposomes.

In some embodiments, the microparticle has a size comprising 1-1000 micrometers.

A skilled artisan would appreciate that a microparticle containing hundreds of nanoliposome factories, has the capacity for increased regulated production of a cytokine. As used herein, the term “microparticle” may be used interchangeably with the term “microfactory” or “microparticle factory” and the like, having all the same qualities and meanings. Alternatively, a “microparticle” may be used to indicate an untreated microparticle.

In some embodiments, microparticle further comprises one or more superparamagnetic iron oxide nanoparticles. In some embodiments, a superparamagnetic iron oxide nanoparticle (SPION) comprises a particle having a size about 50-200 mm. This addition may in certain embodiments enhance purification of microparticles using methods well known in the art.

Upconversion nanoparticles (UCNPs) are a unique class of optical nanomaterials doped with lanthanide ions featuring a wealth of electronic transitions within the 4f electron shells. These nanoparticles can up-convert two or more lower-energy photons into one high-energy photon (Nature Communications (2018) 9:2415 DOI: 10.1038/s41467-018-04813-5). Practically speaking, upconversion allows infrared (IR) light, which is tissue penetrating, to be converted to blue/UV light, which activates the synthesis. (μF-1-μF-4 and Bulk-1 and Bulk-2 are described in TABLE 1.) For any aspects of the invention utilizing ultraviolet (UV) light, by incorporation of UCNPs into the microparticles, IR light can be used for activation.

In some embodiments, IR light is used. These embodiments are useful where tissue damage by UV light (including, but not limited to, ocular or dermal uses or uses on other tissues known to be particularly susceptible to cancer development due to exposure to UV light).

In some embodiments, microparticles further comprise upconversion nanoparticles, such as, but not limited to, μF-1 through μF-4 and Bulk-1 and Bulk-2, the preparation whereof is described herein (e.g., by the microfluidic droplet generator (“μF”) described herein (for μF-1 through μF-4) or in a bulk mixing process (Bulk-1, Bulk-2). This allows the use of IR light to activate the caged ATP and initiate synthesis within the nanoliposomes.

In some embodiments, microparticles may be encapsulated by a coating. In some embodiments, coatings provide microparticles with enhanced biological characteristics, including interactions with cells and biomolecules. In some embodiments, microparticles are encapsulated with a coating comprising a mix of alginate-heparin. In some embodiments, an alginate-heparin coating may be sulfated.

In some embodiments, microparticles may comprise a “coating” material. In some embodiments, these materials provide microparticles with enhanced biological characteristics, including interactions with cells and biomolecules. In some embodiments, microparticles are formed in the presence of a mix of alginate-heparin. In some embodiments, microparticles are formed in the presence of a mix of alginate. In some embodiments, an alginate may be sulfated. Various kinds of materials can be used to coat the alginate-based particles (https://onlinelibrary.wiley.com/doi/abs/10.1002/adma.201703178). Some non-limiting examples include coatings comprising chitosan, poly(L-lysine), polyethylenimine, or other biocompatible polymers. In some embodiments, the coating material comprises chitosan.

A skilled artisan would appreciate that a description of a microparticle comprising an alginate or alginate-heparin coating may in certain embodiments, encompass a microparticle prepared in the presence of alginate or alginate and heparin, wherein these molecules and integral components of the microparticle synthesized.

In some embodiments, the microfactories produced herein comprise alginate microparticles. In some embodiments, the microfactories produced herein comprise alginate-heparin microparticles. In some embodiments, the microfactories produced herein comprise alginate microparticles coated with lipid bilayer. In some embodiments, the microfactories produced herein comprise alginate-heparin microparticles coated with lipid bilayer.

In some embodiments, a microparticle is encapsulated by a coating, for example but not limited to a microparticle alginate-heparin, further comprising a lipid membrane coating. A wide variety of lipid coatings are known in the art, and may be used in certain embodiments (https://avantilipids.com/tech-support/liposome-preparation/lipids-for-liposome-formation). In some embodiments, the lipid membrane comprises 1-palmitoyl-2-oleoyl-sn-glycero-3-phosphocholine (POPC; IUPAC [(2R)-3-hexadecanoyloxy-2-[(Z)-octadec-9-enoyl]oxypropyl]2-(trimethylazaniumyl)ethyl phosphate).

In some embodiments, microparticles may be targeted to T cells. In some embodiments, a microparticle coat comprises biomolecules that recognize and bind cell surface markers on T cells. In some embodiments, cell surface markers on T cells include CD3 and CD28. In some embodiments, a biomolecule that recognized a T cell surface marker comprises an antibody or a fragment thereof.

### Methods of Use

The nanoliposome factories and microparticle factories described above in detail, and exemplified in Examples 1-3, may in certain embodiments be used for therapeutic treatments, for example but not limit to cancer or tumor therapy. Administration of these “factories” provides a regulatory source of cytokines that may in certain embodiments, beneficially regulate an immune response against a cancer. Thus, these nanoliposome factories and microparticle factories may also be used to regulate the immune response in a subject in need, therapy enhancing therapy, for example but not limited to a cancer or tumor therapy.

In some embodiments, disclosed herein is a method of treating cancer or a tumor in a subject in need, said method comprising administering synthetic nanoliposomes or microparticles to said subject, adjacent to a solid tumor; and exposing the site of administration to UV or IR light.

Caged photosensitive ATP may be “released” upon exposure to ultraviolet (UV) light. In some embodiments, UV light exposure comprises exposure to light comprising about 360-480 nm. In some embodiments, UV light exposure comprises exposure to light comprising about 365 nm. In some embodiments, exposure is for a set time period. In some embodiments, exposure comprises a time period of between about 10-300 seconds. In some embodiments, exposure comprises a time period of about 10, 20, 30, 40, 50, 60, 70, 80, 90, 100, 125, 150, 175, 200, 225, 250, 275, or 300 seconds. As noted herein, by using upconversion nanoparticles (UCNPs) in the microparticles, infrared (IR) light may be used to activate the nanoparticles by the conversion of IR light into UV light. Any embodiments or aspects of the invention utilizing UV light can be used with IR light, with the use of UCNPs. Infrared radiation (IR), sometimes called infrared light, is electromagnetic radiation (EMR) with longer wavelengths than those of visible light. In some embodiments, IR light exposure comprises exposure to light comprising up to about 1.4 mm wavelength (IR-A). In some embodiments, IR light exposure comprises exposure to light comprising about 700 nm-1 mm.

In some embodiments, “treating” comprises therapeutic treatment including prophylactic or preventive measures, wherein the object is to prevent or lessen the targeted pathologic condition or disorder, for example to treat or prevent cancer. Thus, in some embodiments, treating may include directly affecting or curing, suppressing, inhibiting, preventing, reducing the severity of, delaying the onset of, reducing symptoms associated with cancer or a combination thereof. Thus, in other embodiments, treating may include directly affecting or curing, suppressing, inhibiting, preventing, reducing the severity of, delaying the onset of, reducing symptoms associated with a non-cancerous tumor or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “cancer” is one of a group of diseases characterized by uncontrollable growth and having the ability to invade normal tissues and to metastasize to other parts of the body. Cancers have many causes, including, but not limited to, diet, alcohol consumption, tobacco use, environmental toxins, heredity, and viral infections. In most instances, multiple genetic changes are required for the development of a cancer cell. Progression from normal to cancerous cells involves a number of steps to produce typical characteristics of cancer including, e.g., cell growth and division in the absence of normal signals and/or continuous growth and division due to failure to respond to inhibitors thereof; loss of programmed cell death (apoptosis); unlimited numbers of cell divisions (in contrast to a finite number of divisions in normal cells); aberrant promotion of angiogenesis; and invasion of tissue and metastasis.

A “pre-cancerous” condition, lesion, or tumor is a condition, lesion, or tumor comprising abnormal cells associated with a risk of developing cancer. Non-limiting examples of pre-cancerous lesions include colon polyps (which can progress into colon cancer), cervical dysplasia (which can progress into cervical cancer), and monoclonal monopathy (which can progress into multiple myeloma). Premalignant lesions comprise morphologically atypical tissue which appears abnormal when viewed under the microscope, and which are more likely to progress to cancer than normal tissue.

A “non-cancerous tumor” or “benign tumor” is one in which the cells demonstrate normal growth, but are produced, e.g., more rapidly, giving rise to an “aberrant lump” or “compact mass,” which is typically self-contained and does not invade tissues or metastasize to other parts of the body. Nevertheless, a non-cancerous tumor can have devastating effects based upon its location (e.g., a non-cancerous abdominal tumor that prevents pregnancy or causes a ureter, urethral, or bowel blockage, or a benign brain tumor that is inaccessible to normal surgery and yet damages the brain due to unrelieved pressure as it grows).

In some embodiments, methods disclosed herein treat a cancer or a pre-cancerous or non-cancerous tumor. In some embodiments, disclosed herein is a method of treating cancer in a subject in need thereof, comprising the step of administering to said subject synthetic nanoliposomes or microparticles, as described above, into or adjacent to a cancer; and exposing the site to UV light. In some embodiments, a cancer comprises a solid tumor. In some embodiments, the solid tumor is selected from the group comprising any tumor of cellular or organ origin including a tumor of unknown origin; any peritoneal tumor either primary or metastatic; a tumor of gynecological origin or gastrointestinal origin or pancreatic origin or blood vessel origin, any solid tumor, i.e. adenocarcinoma, hematological solid tumor, melanoma etc. In some embodiments, a solid tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma. In another related aspect, the tumor or cancer comprises a metastasis of a tumor or cancer.

In some embodiments, a solid tumor treated using a method described herein, originated as a blood tumor or diffuse tumor.

In some embodiments, disclosed herein is a method of regulating an immune response at the site of a tumor, said method comprising administering synthetic nanoliposomes or microparticles to said subject, adjacent to a solid tumor; and exposing the site of administration to UV light; wherein said regulating the immune response comprises increases proliferation of cytotoxic T cells; increases proliferation of helper T cells; maintains the population of helper T cells at the site of said tumor; activated cytotoxic T cells at the site of said tumor; or any combination thereof.

In some embodiments, administration comprises injection and/or infusion directly into a solid tumor. In some embodiments, administration comprises injection and/or infusion adjacent to a solid tumor. Nanoliposomes or microparticles for injection may be in the form of a pharmaceutical composition formulated as a sterile injectable solution.

In some embodiments, injection comprises subcutaneous injection. In some embodiments, administration comprises infiltrating a tissue adjacent to a solid tumor with nanoliposomes or microparticles, or compositions thereof. In some embodiments, the nanoliposome or microparticle is administered via a guided catheter comprising a UV or IR light source. In some embodiments, the composition is administered, in a non-limiting example, together with angioplasty (e.g., a balloon catheter) or another clot removal treatment.

In some embodiments, “treating” comprises therapeutic treatment including prophylactic or preventive measures, wherein the object is to prevent or lessen the targeted pathologic condition or disorder, for example to treat or prevent an autoimmune disease, an allergic reaction, a localized infection or an infectious disease, an injury or other damage, a transplant or other surgical site, or a symptom thereof, or a combination thereof. Thus, in some embodiments, treating may include directly affecting or curing, suppressing, inhibiting, preventing, reducing the severity of, delaying the onset of, reducing symptoms associated with an autoimmune disease, an allergic reaction, a localized infection or an infectious disease, an injury or other damage, a transplant or other surgical site, or a symptom thereof, or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “focus of interest” a “localized environment,” or a “localized site” comprises a site in which the disease, reaction, infection, injury, or other medical condition is specific to one part or area of the body; in which a symptom or condition of the medical condition is specific to one part or area of the body; or in which treatment is desired for one part or area of the body (even if the disease, reaction, infection, injury, or other medical condition affects other parts or areas of the body or the body as a whole).

In some embodiments, methods disclosed herein treat a focus of interest of an autoimmune disease, an allergic reaction, a localized site of an infection or infectious disease, a localized site of an injury or other damage, a transplant or other surgical site, or a symptom thereof, or a combination thereof. In some embodiments, disclosed herein is a method of treating a focus of interest of an autoimmune disease, an allergic reaction, a localized infection or an infectious disease, an injury or other damage, a transplant or other surgical site, or a symptom thereof, or a combination thereof, in a subject in need thereof, comprising the step of administering to said subject synthetic nanoliposomes or microparticles, as described above, into or adjacent to a site of an autoimmune disease, a localized infection or an infectious disease, an injury or other damage, a transplant or other surgical site; a blood clot causing or at risk for causing a myocardial infarction, an ischemic stroke, or a pulmonary embolism; and exposing the site to UV light or to IR light.

In some embodiments, an autoimmune disease, an allergic reaction, a localized infection or an infectious disease, an injury or other damage, a transplant or other surgical site, or a symptom thereof, or a combination thereof, comprises a localized site of an autoimmune disease or allergic reaction, a localized site of an infection or infectious disease, a localized site of injury or damage, a transplant or other surgical site, a blood clot causing or at risk for causing a myocardial infarction, an ischemic stroke, or a pulmonary embolism, or another site comprising one or more localized symptoms thereof, or a combination thereof.

In some embodiments, the autoimmune disease includes, for example, but is not limited to, rheumatoid arthritis, juvenile dermatomyositis, psoriasis, psoriatic arthritis, sarcoidosis, lupus, Crohn's disease, eczema, vasculitis, ulcerative colitis, multiple sclerosis, or type 1 diabetes, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital hear block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis) giant cell myocarditis, glomerulonephritis, Goodpasture's syndrome, granulomatosis with polyangiitis, Grave's disease, Guillain-Barre syndrome, Hashimoto's thyroiditis, hemolytic anemia, Henoch-Schonlein purpura (HSP), Herpes gestationis or pemphigoid gestationis (PG), Hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease, lupus, Lyme disease chronic, Menier's disease, microscopic polyangiitis (MPA), mixed connective tissue disease (MCTD), Mooren's ulcer, Mucha-Habermann disease, multifocoal motor neuropathy (MMN, MMNCB), multiple sclerosis, myasthenia gravis, myositis, narcolepsy, neonatallupus, neuromyelitis optica, neutropenia, ocular cicatricial pemphigoid, optic neuritis, palindromic rheumatism (PR), PANDAS, paraneoplasticcerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes types I-III, polymyalgia rheumatica, polymyositis, postmyocadial infarction syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy (RSD; complex regional pain syndrome [CRPS]), relapsing polychondritis, restless leg syndrome (RLS), retroperitoneal fibrosis, rheumatic fever, rheumatoid arthritis (RA), sarcoidosis, Schmidt syndrome, scleritis, scleroderma, Sjörgren's syndrome, sperm & testicular autoimmunity, stiff person syndrome (SPS), subacute bacterial endocarditis (SBE), Susac's syndrome, sympathetic ophthalmia (SO), Takayasu arteritis, temporal arteritis/giant cell arteritis, thrombocytopenic purpura (TTP), thyroid eye disease (TED), Tolosa-Hunt syndrome (THS), transverse myelitis, type 1 diabetes, ulcerative colitis (UC), undifferentiated connective tissue disease (UCTD), uveitis, vasculitis, vitiligo, or Vogt-Koyanagi-Harada disease. In some embodiments, the localized site of an autoimmune disease includes, for example, but is not limited to, a joint or other area with inflammation, pain or damage from rheumatoid arthritis; an area affected by juvenile dermatomyositis; psoriatic rash or a joint or other area with psoriatic inflammation; a dermal or other region with symptoms of lupus or eczema; a vascular region damaged by vasculitis; an area of myelin sheath damaged by multiple sclerosis; or a pancreatic islet damaged by type 1 diabetes.

Alternatively, protein production locally for autoimmune diseases targets the pathogenic antibodies in the disease, for example, a protein that breaks down antibodies in the vicinity (an IgG endopeptidase) or a protein that binds antibodies (a decoy of the antibody's autoimmune target).

In some embodiments, the allergic reaction includes, for example, but is not limited to, a localized allergic reaction or hypersensitivity reaction including a skin rash, hives, localized swelling (e.g., from an insect bite), esophageal inflammation from food allergies or eosinophilic esophagitis, other enteric inflammation from food allergies or eosinophilic gastrointestinal disease, localized drug allergies when the drug treatment was local to a part of the body, or allergic conjunctivitis.

In some embodiments, the localized site of an infection or the localized site of an infectious disease includes, for example, but is not limited to, a fungal infection (e.g., aspergillus, coccidioidomycosis), a bacterial infection (e.g., methicillin-resistant Staphylococcus aureus, localized skin infections, abscesses, necrotizing facsciitis, pulmonary bacterial infections [e.g., pneumonia], bacterial meningitis, bacterial sinus infections), a viral infection (e.g., varicella-zoster/herpes zoster [shingles], Herpes simplex I [e.g., cold sores/fever blisters], Herpes simplex II [genital herpes], human papilloma virus [e.g., cervical cancer, throat cancer, esophageal cancer, mouse cancer], Epstein-Barr virus [e.g., nasopharyngeal cancer], encephalitis viruses [e.g., brain inflammation], or hepatitis viruses [e.g., liver disease; hepatitis A, hepatitis B, hepatitis C, hepatitis D, hepatitis E, hepatitis F, hepatitis G]), or a parasitic infection (e.g., an area infected by scabies, Chagas, Hypoderma tarandi, amoebae, roundworm, or Toxoplasma gondii).

In some embodiments, the injury or other damage includes, for example, but is not limited to traumatic injury (e.g., resulting from an accident or violence) or chronic injury (e.g., osteoarthritis). In some embodiments, the localized site of injury comprises a muscular-skeletal injury, a neurological injury, an eye or ear injury, an internal or external wound, a localized abscess, an area of mucosa that is affected (e.g., conjunctiva, sinuses, esophagus), or an area of skin that is affected (e.g., infection, autoimmunity. In some embodiments, the transplant or other surgical site includes, for example, but is not limited to, the site and/or its local environment or surroundings of an organ, corneal, skin, limb, face, or other transplant, or a surgical site and/or its local environment or surroundings, for, e.g., but not limited to, treatment of surgical trauma, treatment of a condition related to the transplant or surgery, or prevention of infection. In some embodiments, the site is at or adjacent to a blood clot causing or at risk for causing a myocardial infarction, an ischemic stroke, or a pulmonary embolism.

In some embodiments, the nanoliposome or microparticle is administered via a guided catheter comprising a UV or IR light source. In some embodiments, the methods disclosed herein treat one or more symptoms of a disease, reaction, infection, injury, transplant, surgery, or blood clot. In some embodiments, the methods disclosed herein treat a combination thereof.

In some embodiments, disclosed herein is a method of regulating an immune response at the localized site of disease, injury, damage, autoimmune or allergic reaction, or other symptom, including, but not limited to, a localized site of an autoimmune disease, a localized infection or an infectious disease, an injury or other damage, a transplant or other surgical site, said method comprising: administering synthetic nanoliposomes or microparticles to said subject, adjacent to the localized site of an autoimmune disease, a localized infection or an infectious disease, an injury or other damage, a transplant or other surgical site; and exposing the site of administration to UV or IR light; wherein said regulating the immune response comprises increases proliferation of cytotoxic T cells; increases proliferation of helper T cells; maintains the population of helper T cells at the site of said localized site of an autoimmune disease, a localized infection or an infectious disease, an injury or other damage, a transplant or other surgical site; activated cytotoxic T cells at the site of said localized site of an autoimmune disease, a localized infection or an infectious disease, an injury or other damage, a transplant or other surgical site, a blood clot causing or at risk for causing a myocardial infarction, an ischemic stroke, or a pulmonary embolism, or any combination thereof.

In some embodiments, administration comprises injection and/or infusion directly into a localized site of an autoimmune disease, an allergic reaction, a localized infection or an infectious disease, an injury or other damage, a transplant or other surgical site, or a symptom thereof, a blood clot, or a combination thereof. In some embodiments, administration comprises injection and/or infusion adjacent to a localized site of an autoimmune disease, an allergic reaction, a localized infection or an infectious disease, an injury or other damage, a transplant or other surgical site, a blood clot, or a symptom thereof, or a combination thereof. Nanoliposomes or microparticles for injection may be in the form of a pharmaceutical composition formulated as a sterile injectable solution.

In some embodiments, injection comprises subcutaneous injection. In some embodiments, administration comprises infiltrating a tissue adjacent to a localized site of an autoimmune disease, an allergic reaction, a localized infection or an infectious disease, an injury or other damage, a transplant or other surgical site, a blood clot, or a symptom thereof, or a combination thereof, with nanoliposomes or microparticles, or compositions thereof.

In some embodiments, the nanoliposome or microparticle is administered via a guided catheter comprising a UV or IR light source.

In some embodiments, injection comprises injecting a microparticle not comprising alginate, heparin, or a lipid coating. In some embodiments, injection comprises injecting a microparticle comprising alginate. In some embodiments, injection comprises injecting a microparticle comprising alginate-heparin. In some embodiments, injection comprises injecting a microparticle comprising alginate and a lipid coating. In some embodiments, injection comprises injecting a microparticle comprising alginate-heparin and a lipid coating. In certain embodiments, these microparticles can be injected through the blood. In certain embodiments, these microparticles can be injected through locally at the site of a tumor. Encapsulation into microparticles provides in some embodiments, further control over the release of the cytokine expressed, and also localizes the effects. In some embodiments, injection nanoliposomes from inside microparticles provides a stronger cytokine gradient to boost up the therapeutic effects.

An additional advantage of methods described herein is the use of a caged ATP molecule, wherein the release of ATP by, for example, UV light controls the transcription and translation of the encoded cytokine, thereby providing a regulatable expression of a beneficial cytokine in a localized region at or adjacent to a tumor.

In some embodiments, application of nanoliposomes or microparticles, or compositions thereof is for local use. This may, in certain embodiments, provide an advantage, wherein the expressed cytokine may provide a local immune effect thereby avoiding a toxic systemic effect of the cytokine. In one example, controlled expression and release of IL-2 or an IL-2 superkine, may increase proliferation of cytotoxic T cells and or helper T cells in the area adjacent to the cancer or tumor, thereby promoting clearance of the cancer or tumor. In some embodiments, controlled expression and release of IL-2 or an IL-2 superkine, may maintain a helper T cell population in the area adjacent to the tumor. In some embodiments, controlled expression and release of IL-2 or an IL-2 superkine, may activate a cytotoxic T cell population in the area adjacent to the tumor. In some embodiments, controlled expression and release of IL-2 or an IL-2 superkine, may lead to enhanced killing of tumor cells in the localized area at and adjacent to the tumor. In some embodiments, controlled expression and release of IL-2 or an IL-2 superkine, provides enhanced clearance of a tumor. In another example, localized infection of the cervix with human papilloma virus (HPV) is treated with localized release of IL-15 cytokines to promote cytotoxic T cells to attack the virally infected tissues. In still another example, an autoimmune skin disease (e.g., vitiligo or alopecia) is treated by localized release of inhibitory cytokines (e.g., IL-4 or IL-10) to slow down the immune damage. In yet another example, an autoimmune disease (e.g., type 1 diabetes) is treated by localized release of inhibitory cytokines (e.g., IL-4 or IL-10) to slow down the immune damage (e.g., in pancreatic islets or elsewhere). This technique may also be used for the treatment of other diseases, reactions, injuries, transplants, blood clots, and the like, recited herein.

As used herein, the terms “composition” and “pharmaceutical composition” may in some embodiments, be used interchangeably having all the same qualities and meanings. In some embodiments, disclosed herein is a pharmaceutical composition for the treatment of a cancer or tumor as described herein. In some embodiments, disclosed herein is a pharmaceutical composition for the treatment of cancer or tumor. In some embodiments, disclosed herein is a pharmaceutical composition for the use in methods locally regulating an immune response. In some embodiments, disclosed herein are pharmaceutical compositions for the treatment of an autoimmune disease, an allergic reaction, a localized site of an infection or infectious disease, a localized site of an injury or other damage, a transplant or other surgical site, a blood clot causing or at risk for causing a myocardial infarction, an ischemic stroke, or a pulmonary embolism or a symptom thereof, or a combination thereof.

In some embodiments, a pharmaceutical composition comprises nanoliposomes or microparticles, as described in detail above. In still another embodiment, a pharmaceutical composition for the treatment of cancer or tumor, as described herein, comprises an effective amount of nanoliposomes or microparticles and a pharmaceutically acceptable excipient. In some embodiments, a composition comprising nanoliposomes or microparticles and a pharmaceutically acceptable excipient is used in methods for regulating an immune response. In some embodiments, a composition comprising nanoliposomes or microparticles is used in methods to reduce the size of a tumor. In some embodiments, a composition comprising nanoliposomes or microparticles is used in methods to eliminate the tumor. In some embodiments, a composition comprising nanoliposomes or microparticles is used in methods to slow the growth of a tumor. In some embodiments, a composition comprising nanoliposomes or microparticles is used in methods to prolong the survival of the subject. In some embodiments, methods of treating described herein reduce the size of the tumor, eliminate said tumor, slow the growth of the tumor, or prolong survival of said subject, or any combination thereof.

In some embodiments, a pharmaceutical composition comprises nanoliposomes or microparticles, as described in detail above. In still another embodiment, a pharmaceutical composition for the treatment of an autoimmune disease, an allergic reaction, a localized site of an infection or infectious disease, a localized site of an injury or other damage, a transplant or other surgical site, a blood clot, or a symptom thereof of any one of these, or a combination thereof, as described herein, comprises an effective amount of nanoliposomes or microparticles and a pharmaceutically acceptable excipient. In some embodiments, a composition comprising nanoliposomes or microparticles and a pharmaceutically acceptable excipient is used in methods for regulating an immune response. In some embodiments, a composition comprising nanoliposomes or microparticles is used in methods for promoting clearance of or alleviating localized symptoms of the autoimmune disease, allergic reaction, infection or infectious disease. In some embodiments, a composition comprising nanoliposomes or microparticles is used in methods for facilitating healing and/or preventing or inhibiting infection or rejection of a localized site of an injury or other damage, a transplant or other surgical site. In some embodiments, a composition comprising nanoliposomes or microparticles is used in methods for alleviating localized symptoms relating to an autoimmune disease, an allergic reaction, a localized site of an infection or infectious disease, a localized site of an injury or other damage, a transplant or other surgical site, or a symptom thereof, or a combination thereof. In some embodiments, a composition comprising nanoliposomes or microparticles is used in methods to prolong the survival of the subject. In some embodiments, methods of treating described herein for promoting clearance of or alleviating localized symptoms of the autoimmune disease, allergic reaction, infection or infectious disease; for facilitating healing and/or preventing or inhibiting infection or rejection of a localized site of an injury or other damage, a transplant or other surgical site; for reducing or eliminating a blood clot causing or at risk for causing a myocardial infarction, an ischemic stroke, or a pulmonary embolism; or for alleviating localized symptoms thereof; or for a combination thereof.

In some embodiments, a method of use of nanoliposomes or microparticles further comprises a step of administering activated T cells to said subject. Methods of preparing T cells are known in the art. In some embodiments, these cells may be administered prior to or after administering said nanoliposome or said microparticle. In other embodiments, administering activated T cells is prior to or after exposing the site to UV light. In some embodiments, T cells are administered by intravenous (i.v.) injection. In some embodiments, administration of T cells enhances the therapeutic effect provided by the regulated, local expression of a cytokine from administered nanoliposomes or microparticles.

### Therapeutic and Diagnostic Proteins, Polypeptides and Peptides

In related aspects to the foregoing description, protein production by the transcription an translation system of the nanoliposomes may be used for production of non-cytokine therapeutic proteins and diagnostic proteins or polypeptides, useful for therapeutic and diagnostic purposes where systemic administration of the protein, polypeptide or peptide is ineffective, toxic, or otherwise contraindicated. All other aspects of the composition, components and preparation of the nanoliposomes and microparticles are the same as described herein. The plasmid comprises a nucleic acid encoding a protein, polypeptide or peptide other than a cytokine, such as, but not limited to, chemokine, a therapeutic antibody or a fragment thereof or antigen-binding protein, a Fc fusion protein, an anticoagulant, an enzyme, a hormone, or a thrombolytic. In one embodiment, the enzyme is a “clot-buster,” a thrombolytic therapy used to break up blood clots, such as, but not limited to, those causing, e.g., an infarction of a blood vessel (such as, but not limited to, a heart attack [myocardial infarction], an ischemic stroke, or a pulmonary embolism [PE]). Examples of “clot-buster” thrombolytics include, but are not limited to, tissue plasminogen activator (tPA), tenecteplase, alteplase, urokinase, reteplase, and streptokinase. In some embodiments, the composition is administered, in a non-limiting example, together with angioplasty (e.g., a balloon catheter) or another clot removal treatment.

In some embodiments, the composition is administered via a guided catheter with a UV or IR light. This approach is particularly useful for treatment of non-surface or inaccessible tumors, clots, injuries, transplants, surgical sites, or other medical conditions (e.g., crossing the blood-brain barrier). In some embodiments, the composition is administered via a guided catheter separate from the source of UV or IR light.

In some embodiments, the therapeutic protein is a multimer wherein the individual protein or polypeptide subunits are produced and association into the active protein occurs within or outside of the nanofactories or microparticles. In one embodiment, microparticles comprising nanofactories capable of producing a therapeutic protein are infused into a patient and the production of the therapeutic protein activated by exposure to the microparticles at a desired site in the body to UV, IR, or other electromagnetic radiation that uncages ATP and initiates synthesis of the protein as described here. The site can be at the surface of the body or accessible by catheter or other semi-invasive means, or by surgery to activate the nanofactories at the desired site. In another embodiment, the microparticles can be administered systemically but only activated at the desired site. Activation of production of the therapeutic protein provides therapeutic benefit to the subject.

As used herein, a “targeting agent,” or “affinity reagent,” is a molecule that binds to an antigen or receptor or other molecule. In some embodiments, a “targeting agent” is a molecule that specifically binds to an antigen or receptor or other molecule. In certain embodiments, some or all of a targeting agent is composed of amino acids (including natural, non-natural, and modified amino acids), nucleic acids, or saccharides. In certain embodiments, a “targeting agent” is a small molecule.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, C-gamma-1 (Cγ1), C-gamma-2 (Cγ2), and C-gamma-3 (Cγ3). In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. As used herein, the term “antibody” refers to a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes or classes, respectively.

The term “antibody” is meant to include full-length antibodies, and may refer to a natural antibody from any organism, an engineered antibody, or an antibody generated recombinantly for experimental, therapeutic, or other purposes as further defined below. Furthermore, full-length antibodies comprise conjugates as described and exemplified herein. As used herein, the term “antibody” comprises monoclonal and polyclonal antibodies. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e. bi-specific) hybrid antibodies (see, e.g., Lanzavecchia et al., Eur. J. Immunol. 17, 105 (1987)) and in single chains (see, e.g., Huston et al., Proc. Natl. Acad. Sci. U.S.A., 85, 5879-5883 (1988) and Bird et al., Science, 242, 423-426 (1988)). (See, generally, Hood et al., “Immunology”, Benjamin, N.Y., 2nd ed. (1984), and Hunkapiller and Hood, Nature, 323, 15-16 (1986)).

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

In one embodiment, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tri(a)body, a di- or tri-tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “BY” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (see, e.g., Spatola et al., “Chemistry and Biochemistry of Amino Acids, Peptides and Proteins,” B. Weinstein, eds., Marcel Dekker, New York, p. 267 (1983); Morley, J. S., “Trends Pharm Sci.” (1980) pp. 463-468; Hudson et al., Int. J. Pept. Prot. Res. (1979) 14, 177-185; Spatola et al., Life Sci. (1986) 38, 1243-1249; Hann, M. M., J. Chem. Soc. Perkin Trans. I (1982) 307-314; Almquist et al., J. Med. Chem. (1980) 23, 1392-1398; Jennings-White et al., Tetrahedron Lett. (1982) 23, 2533; Szelke et al., European Application EP 45665; Chemical Abstracts 97, 39405 (1982); Holladay, et al., Tetrahedron Lett. (1983) 24, 4401-4404; and Hruby, V. J., Life Sci. (1982) 31, 189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10-5 M or less than about 1×10-6 M or 1×10-7 M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In addition to antibody sequences, an antibody according to the present invention may comprise other amino acids, e.g., forming a peptide or polypeptide, such as a folded domain, or to impart to the molecule another functional characteristic in addition to ability to bind antigen. For example, antibodies of the invention may carry a detectable label, such as fluorescent or radioactive label, or may be conjugated to a toxin (such as a holotoxin or a hemitoxin) or an enzyme, such as beta-galactosidase or alkaline phosphatase (e.g., via a peptidyl bond or linker).

In one embodiment, an antibody of the invention comprises a stabilized hinge region. The term “stabilized hinge region” will be understood to mean a hinge region that has been modified to reduce Fab arm exchange or the propensity to undergo Fab arm exchange or formation of a half-antibody or a propensity to form a half-antibody. “Fab arm exchange” refers to a type of protein modification for human immunoglobulin, in which a human immunoglobulin heavy chain and attached light chain (half-molecule) is swapped for a heavy-light chain pair from another human immunoglobulin molecule. Thus, human immunoglobulin molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half-antibody” forms when a human immunoglobulin antibody dissociates to form two molecules, each containing a single heavy chain and a single light chain. In one embodiment, the stabilized hinge region of human immunoglobulin comprises a substitution in the hinge region.

In one embodiment, the term “hinge region” as used herein refers to a proline-rich portion of an immunoglobulin heavy chain between the Fc and Fab regions that confers mobility on the two Fab arms of the antibody molecule. It is located between the first and second constant domains of the heavy chain. The hinge region includes cysteine residues which are involved in inter-heavy chain disulfide bonds. In one embodiment, the hinge region includes cysteine residues which are involved in inter-heavy chain disulfide bonds.

In one embodiment, the antibody or antigen-binding fragment binds its target with a KD of 0.1 nM-10 mM. In one embodiment, the antibody or antigen-binding fragment binds its target with a KD of 0.1 nM-1 mM. In one embodiment, the antibody or antigen-binding fragment binds its target with a KD within the 0.1 nM range. In one embodiment, the antibody or antigen-binding fragment binds its target with a KD of 0.1-2 nM. In another embodiment, the antibody or antigen-binding fragment binds its target with a KD of 0.1-1 nM. In another embodiment, the antibody or antigen-binding fragment binds its target with a KD of 0.05-1 nM. In another embodiment, the antibody or antigen-binding fragment binds its target with a KD of 0.1-0.5 nM. In another embodiment, the antibody or antigen-binding fragment binds its target with a KD of 0.1-0.2 nM.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises an C-terminal modification that allows binding to an enzymatically biotinylatable site. In some embodiments, biotinylation of said site functionalizes the site to bind to any surface coated with streptavidin, avidin, avidin-derived moieties, or a secondary reagent.

It will be appreciated that the term “modification” can encompass an amino acid modification such as an amino acid substitution, insertion, and/or deletion in a polypeptide sequence.

In one embodiment, a variety of radioactive isotopes are available for the production of radioconjugate antibodies and other proteins and can be of use in the methods and compositions provided herein. Examples include, but are not limited to, At211, Cu64, I131, I125, Y90, Re186, Re188, Sm153, Bi212, P32, Zr89 and radioactive isotopes of Lu. In a further embodiment, the amino acid sequences of the invention may be homologues, variants, isoforms, or fragments of the sequences presented. The term “homolog” as used herein refers to a polypeptide having a sequence homology of a certain amount, namely of at least 70%, e.g. at least 80%, 90%, 95%, 96%, 97%, 98%, 99% of the amino acid sequence it is referred to. Homology refers to the magnitude of identity between two sequences.

Homolog sequences have the same or similar characteristics, in particular, have the same or similar property of the sequence as identified. The term ‘variant’ as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g. as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide of the invention which has less amino acids than the full-length amino acid sequence of a polypeptide of the invention. The fragment may or may not possess a functional activity of such polypeptides.

In an alternate embodiment, enzymatically active toxin or fragments thereof that can be used in the compositions and methods provided herein include, but are not limited, to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

A chemotherapeutic or other cytotoxic agent may be conjugated to the protein, according to the methods provided herein, as an active drug or as a prodrug. The term “prodrug” refers to a precursor or derivative form of a pharmaceutically active substance that is less cytotoxic to tumor cells compared to the parent drug and is capable of being enzymatically activated or converted into the more active parent form. (See, e.g., Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.) The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

Non-limiting examples of antibodies, antibody fragments and antigen-binding proteins include single-chain antibodies such as scFvs, that can be synthesized at a desired site in the body accessible to microparticles via circulation, subcutaneous injection, or delivery at or near the desired site. A non-limiting example, a scFv that blocks PD-1 for the treatment of cancer or tumor, including in association with CAR-T therapy, wherein activation of scFv production can be directed at a particular site in the body, in one embodiment, at or near a tumor. Another non-limiting example includes brolucizumab, which targets VEGF-A and is used to treat wet age-related macular degeneration. In one embodiment, production of this scFv at the macula can be initiated by exposing IR light to the site thought the eye after infusion of microparticles comprising nanofactories and upconversion nanoparticles into the vitreous or via circulation. Another non-limiting example of a therapeutic protein being made locally is a defensin (cathelecidin) or another antimicrobial peptide (e.g., REG3G). Still other non-limiting examples of therapeutic proteins being made locally include fragments of proteins that bind or “decoy” pathogenic proteins. Yet other non-limiting examples of therapeutic proteins being made locally are antagonists of signaling (e.g., the IL-1 receptor antagonist [genomic IL 1RA, anakinra]).

In another example, the therapeutic protein is an immune checkpoint inhibitor, such as an antibody fragment, or antigen-binding protein, that inhibits a checkpoint molecule such as but not limited to PD-1, PD-L1, CTLA-4, CTLA-4 receptor, PD1-L2, 4-1BB, OX40, LAG-3 and TIM-3. In one embodiment, a scFv that inhibits a checkpoint protein is produced by nanofactories upon activation. In one embodiment microparticles comprising such nanofactories are used in association with a cancer or tumor therapy, such as CAR-T therapy. Thus, the microparticles that can produce such inhibitory proteins provide a similar therapeutic activity as antibodies to PD-1 and other checkpoint molecules, which, in one embodiment, are produced when and where UV or IR light is exposed to the microparticles.

“Apheresis” comprises an ex vivo blood purification procedure during which a patient's blood is subjected to a separation apparatus or technique ex vivo to separate out a given constituent prior to the reinfusion of the blood back into the patient (or a different patient). “Leukapheresis” comprises apheretic separation of leukocytes from the blood.

In one embodiment microparticles can be targeted to and be bound to T cells during a leukapheresis or other blood cell purification procedure and infused into the patient. Such targeting binding of microparticles to lymphocytes or other cell types after administration to the body or during a leukapheresis procedure or other ex vivo procedure provides the therapeutic protein in association with a cell type to effect its desired function when activated by UV or IR light.

In another example, activation of chemokine production at a desired focal site may attract immune cells to treat cancer or a tumor, localized inflammatory disease or site of an infection. In another example, activation of chemokine production at a desired site may attract immune cells to treat an autoimmune disease, an allergic reaction, a localized site of an infection or infectious disease, a localized site of an injury or other damage, a transplant or other surgical site, or a symptom thereof, or a combination thereof.

In other embodiments, production of an enzyme or hormone is therapeutically useful at a site in or on the body accessible to UV or IR light.

In another embodiment, a diagnostic protein is produced by the microparticles for use in identifying a site of pathology. In one example, binding of the therapeutic protein at a target site can then be identified by imaging or other means.

In another embodiment, microparticles of the invention may be modified to bind or target certain cell types, such as T lymphocytes as described above, such that activation releases a therapeutic protein such as an antibody or antigen-binding protein, e.g., a scFv, that activates or enhances the activity of the T lymphocyte in some embodiments to increase activity; in other embodiments to decrease activity; and in other embodiments to induce formation of T regulatory cells.

In some embodiments, the methods are used for the treatment of vertebrate organisms. In some embodiments, the methods are used for the treatment of homeothermic vertebrate organisms (e.g., mammals and birds). In some embodiments, the methods are used for the treatment of human or non-human mammals.

Any embodiment herein in which activation and production of a cytokine is desirable or beneficial is applicable to activation and production of a non-cytokine therapeutic protein or diagnostic protein.

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

“Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to ±10%.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Any patent, patent application publication, or scientific publication, cited herein, is incorporated by reference herein in its entirety.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Production of Nanoliposomes and Methods of Use

One or more nanoliposomes are produced comprising a cell-free transcription and translation system; a plasmid comprising a nucleic acid encoding a protein of interest; and optionally, a photoactivatable-caged adenosine triphosphate (ATP).

The protein is a therapeutic protein selected for the treatment of a disease or medical condition of interest, or for the alleviation of localized symptoms, or combinations thereof, in a subject. Alternatively, the protein is a diagnostic protein selected for detecting the presence of a disease or medical condition of interest, or a component or indicator thereof, in a subject.

The plasmid encoding the protein is prepared to express the protein via the cell-free transcription and translation system upon activation of the system. Where the nanoliposome comprises a photoactivatable-caged ATP, in response to exposure to an activating light, such as UV light, infrared light (IR) (such as in the presence of upconversion nanoparticles), or another source of electromagnetic radiation, thereby releasing the photoactivatable-caged ATP in order to initiate the processes of transcription and translation of the protein during or after administration of the nanoliposome (or of a microparticle comprising the nanoliposome) to a subject in need thereof. Alternatively, the plasmid expresses the protein without the need for photoactivation.

The nanoliposome is administered at the focus of interest on or within the subject in need thereof and, optionally, is exposed to the activating light so that the photoactivatable-caged ATP is released and initiates the processes of transcription and translation of the protein, which is then expressed at the focus of interest.

The regulatable expression of a therapeutic protein at a focus of interest treats a localized environment on or within the subject in need thereof. Alternatively, the regulatable expression of a diagnostic protein at a focus of interest detects the presence of a disease or medical condition of interest or a component or indicator thereof, on or within the subject in need thereof.

### Example 2: Production of Microparticles and Methods of Use

One or more microparticles are produced comprising at least one nanoliposome, the nanoliposome comprising a cell-free transcription and translation system; a plasmid comprising a nucleic acid encoding a protein of interest; and optionally, a photoactivatable-caged adenosine triphosphate (ATP).

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles.

The protein is a therapeutic protein selected for the treatment of a disease or medical condition of interest, or for the alleviation of localized symptoms, or combinations thereof, in a subject. Alternatively, the protein is a diagnostic protein selected for detecting the presence of a disease or medical condition of interest, or a component or indicator thereof, in a subject.

Where the nanoliposome comprises a photoactivatable-caged ATP, the plasmid encoding the protein is prepared to express the protein via the cell-free transcription and translation system upon activation of the system in response to exposure to an activating light, such as UV light, infrared light (IR) (such as in the presence of upconversion nanoparticles), or another source of electromagnetic radiation, thereby releasing the photoactivatable-caged ATP in order to initiate the processes of transcription and translation of the protein during or after administration of the microparticle to a subject in need thereof. Alternatively, the plasmid expresses the protein without the need for activation.

The microparticle comprising the nanoliposome is administered at the focus of interest on or within the subject in need thereof and, optionally, is exposed to the activating light so that the photoactivatable-caged ATP is released and initiates the processes of transcription and translation of the protein, which is then expressed at the focus of interest. Alternatively, the plasmid expresses the protein without the need for photoactivation.

The regulatable expression of a therapeutic protein at a focus of interest treats a localized environment on or within the subject in need thereof. Alternatively, the regulatable expression of a diagnostic protein at a focus of interest detects the presence of a disease or medical condition of interest or a component or indicator thereof, on or within the subject in need thereof.

### Example 3: Production of Multiple Nanoliposomes and Methods of Use

Two or more nanoliposomes are produced, each comprising a cell-free transcription and translation system; a plasmid comprising a nucleic acid encoding a protein of interest; and optionally, a photoactivatable-caged adenosine triphosphate (ATP), as in Example 1.

However, the protein of interest expressed in each type of nanoliposome is distinct.

Each of the two or more proteins is a therapeutic protein selected for the treatment of a disease or medical condition of interest, or for the alleviation of localized symptoms, or combinations thereof, in a subject and/or is a diagnostic protein selected for detecting the presence of a disease or medical condition of interest, or a component or indicator thereof, in a subject. Alternatively, each of the two or more proteins is a therapeutic protein selected to act synergistically for the treatment of a disease or medical condition or interest and/or for the alleviation of localized symptoms, or combinations thereof.

Each plasmid encoding the protein particular to that plasmid is prepared to express its given protein via the cell-free transcription and translation system upon activation of the system in response to exposure to an activating light, such as UV light, infrared light (IR) (such as in the presence of upconversion nanoparticles), or another source of electromagnetic radiation, thereby releasing the photoactivatable-caged ATP in order to initiate the processes of transcription and translation of the protein during or after administration of the nanoliposome (or of a microparticle comprising the nanoliposome) to a subject in need thereof. Alternatively, one or both of the plasmids expresses its respective protein without the need for photoactivation.

Each type of nanoliposome is administered at the focus of interest on or within the subject in need thereof and is exposed to the activating light. The photoactivatable-caged ATP is released and initiates the processes of transcription and translation of the two or more proteins, which are then expressed at the focus of interest. Different types of nanoliposomes may be administered and/or activated simultaneously or sequentially.

The regulatable expression of a therapeutic protein and/or a diagnostic protein at a focus of interest, respectively, treats a localized environment on or within the subject in need thereof and/or detects the presence of a disease or medical condition of interest or a component or indicator thereof, on or within the subject in need thereof.

### Example 4: Production of Microparticles Having Multiple Nanoliposomes or Multiple Microparticles and Methods of Use

One or more types of microparticles are produced, each comprising two or more types of nanoliposomes, each nanoliposome comprising a cell-free transcription and translation system; a plasmid comprising a nucleic acid encoding a protein of interest; and optionally, a photoactivatable-caged adenosine triphosphate (ATP), as in Examples 1 and 3.

Each microparticle optionally comprises alginate or alginate-heparin, either as a component or as a coating. Each microparticle optionally further comprises a lipid membrane coating, optionally comprising POPC. Each microparticle optionally comprises one or more supermagnetic iron oxide nanoparticles (SPION) and/or one or more upconversion nanoparticles, as in Example 2.

However, the protein of interest expressed in each type of nanoliposome is distinct.

Each of the two or more proteins is a therapeutic protein selected for the treatment of a disease or medical condition of interest, or for the alleviation of localized symptoms, or combinations thereof, in a subject and/or is a diagnostic protein selected for detecting the presence of a disease or medical condition of interest, or a component or indicator thereof, in a subject. Alternatively, each of the two or more proteins is a therapeutic protein selected to act synergistically for the treatment of a disease or medical condition or interest and/or for the alleviation of localized symptoms, or combinations thereof.

Each microparticle comprises each type of nanoliposome, or two or more types of microparticles are used, each type of microparticle comprising a distinct type of nanoparticle. Each type of microparticle may be separately activatable. Alternatively, one or both of the plasmids expresses its respective protein without the need for photoactivation.

Each plasmid encoding the protein particular to that plasmid is prepared to express its given protein via the cell-free transcription and translation system upon activation of the system in response to exposure to an activating light, such as UV light, infrared light (IR) (such as in the presence of upconversion nanoparticles), or another source of electromagnetic radiation, thereby releasing the photoactivatable-caged ATP in order to initiate the processes of transcription and translation of the protein during or after administration of the microparticle comprising the nanoliposome to a subject in need thereof.

Where two or more types of microparticles are administered, each type of microparticle is administered at the focus of interest on or within the subject in need thereof and optionally, is exposed to the activating light so that the photoactivatable-caged ATP is released and initiates the processes of transcription and translation of the two or more proteins, which are then expressed at the focus of interest. Different types of microparticles may be administered and/or activated simultaneously or sequentially. Alternatively, one or both of the plasmids expresses its respective protein without the need for photoactivation.

The regulatable expression of a therapeutic protein and/or a diagnostic protein at a focus of interest, respectively, treats a localized environment on or within the subject in need thereof and/or detects the presence of a disease or medical condition of interest or a component or indicator thereof, on or within the subject in need thereof.

The protein is a therapeutic protein selected for the treatment of a disease or medical condition of interest, or for the alleviation of localized symptoms, or combinations thereof, in a subject. Alternatively, the protein is a diagnostic protein selected for detecting the presence of a disease or medical condition of interest, or a component or indicator thereof, in a subject.

Where the nanoliposome comprises a photoactivatable-caged ATP, the plasmid encoding the protein is prepared to express the protein via the cell-free transcription and translation system upon activation of the system in response to exposure to an activating light, such as UV light, infrared light (IR) (such as in the presence of upconversion nanoparticles), or another source of electromagnetic radiation, thereby releasing the photoactivatable-caged ATP in order to initiate the processes of transcription and translation of the protein during or after administration of the microparticle to a subject in need thereof.

Where the nanoliposome comprises a photoactivatable-caged ATP, the microparticle comprising the nanoliposome is administered at the focus of interest on or within the subject in need thereof and is exposed to the activating light. The photoactivatable ATP is released and initiates the processes of transcription and translation of the protein, which is then expressed at the focus of interest.

The regulatable expression of a therapeutic protein at a focus of interest treats a localized environment on or within the subject in need thereof. Alternatively, the regulatable expression of a diagnostic protein at a focus of interest detects the presence of a disease or medical condition of interest or a component or indicator thereof, on or within the subject in need thereof.

### Example 5: Production of Cytokine-Expressing Nanoliposomes and Microparticles and Methods of Use

One or more nanoliposomes are produced comprising a cell-free transcription and translation system; a plasmid comprising a nucleic acid encoding a cytokine of interest; and optionally, an ultraviolet (UV)-caged or an infrared (IR)-caged adenosine triphosphate (ATP).

One or more microparticles are produced comprising at least one nanoliposome, the nanoliposome comprising a cell-free transcription and translation system; a plasmid comprising a nucleic acid encoding a protein of interest; and optionally, UV-caged or IR-caged adenosine triphosphate (ATP).

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles.

The protein is a cytokine selected for the treatment of a disease or medical condition of interest or for the alleviation of localized symptoms, or combinations thereof, in a subject. The cytokine acts in concert with other proteins or cells to enhance a desired immune response for the treatment of the disease or other medical condition, such as a tumor, infection, or transplant rejection, or for the alleviation of localized symptoms, such as inflammation.

Where the nanoliposome comprises a photoactivatable-caged ATP, the plasmid encoding the protein is prepared to express the cytokine via the cell-free transcription and translation system upon activation of the system in response to exposure to an activating UV light, infrared light (IR) (such as in the presence of upconversion nanoparticles), or another source of electromagnetic radiation, thereby releasing the UV-caged ATP in order to initiate the processes of transcription and translation of the cytokine during or after administration of the nanoliposome (or of a microparticle comprising the nanoliposome) to a subject in need thereof.

Where the nanoliposome comprises a photoactivatable-caged ATP, the microparticle comprising the nanoliposome is administered at the focus of interest on or within the subject in need thereof and is exposed to the activating UV or IR light or other source of electromagnetic radiation. The photoactivatable-caged ATP is released and initiates the processes of transcription and translation of the cytokine, which is then expressed at the focus of interest.

The regulatable expression of a cytokine at a focus of interest treats a localized environment on or within the subject in need thereof.

### Example 6: Production of Other Protein-Expressing Nanoliposomes and Microparticles and Methods of Use

One or more nanoliposomes are produced comprising a cell-free transcription and translation system; a plasmid comprising a nucleic acid encoding a protein of interest (e.g., a chemokine, a therapeutic antibody or fragment thereof, an antigen-binding protein, a Fc fusion protein, an anticoagulant, an enzyme, a hormone, or a thrombolytic); and optionally, an ultraviolet (UV)-caged or an infrared (IR)-caged adenosine triphosphate (ATP).

One or more microparticles are produced comprising at least one nanoliposome, the nanoliposome comprising a cell-free transcription and translation system; a plasmid comprising a nucleic acid encoding a protein of interest (e.g., a chemokine, a therapeutic antibody or fragment thereof, an antigen-binding protein, a Fc fusion protein, an anticoagulant, an enzyme, a hormone, or a thrombolytic); and optionally, a UV-caged or IR-caged adenosine triphosphate (ATP).

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles.

The protein is a protein of interest (e.g., a chemokine, a therapeutic antibody or fragment thereof, an antigen-binding protein, a Fc fusion protein, an anticoagulant, an enzyme, a hormone, or a thrombolytic) selected for the treatment of a disease or medical condition of interest or for the alleviation of localized symptoms, or combinations thereof, in a subject. The protein of interest acts in concert with other proteins or cells to enhance a desired immune response for the treatment of the disease or other medical condition, such as a tumor, infection, or transplant rejection, or for the alleviation of localized symptoms, such as inflammation.

Where the nanoliposome comprises a photoactivatable-caged ATP, the plasmid encoding the protein of interest is prepared to express the protein of interest via the cell-free transcription and translation system upon activation of the system in response to exposure to an activating UV light, infrared light (IR) (such as in the presence of upconversion nanoparticles), or another source of electromagnetic radiation, thereby releasing the UV-caged ATP in order to initiate the processes of transcription and translation of the protein of interest during or after administration of the nanoliposome (or of a microparticle comprising the nanoliposome) to a subject in need thereof.

Where the nanoliposome comprises a photoactivatable-caged ATP, the microparticle comprising the nanoliposome is administered at the focus of interest on or within the subject in need thereof and is exposed to the activating UV or IR light or other source of electromagnetic radiation. The photoactivatable-caged ATP is released and initiates the processes of transcription and translation of the protein of interest, which is then expressed at the focus of interest.

The regulatable expression of a protein of interest (e.g., a chemokine, a therapeutic antibody or fragment thereof, an antigen-binding protein, a Fc fusion protein, an anticoagulant, an enzyme, a hormone, or a thrombolytic) at a focus of interest treats a localized environment on or within the subject in need thereof.

### Example 7: Treatment of Cancerous, Pre-Cancerous, and Non-Cancerous Tumors with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); and optionally, an ultraviolet (UV)-caged or infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and optionally, a UV-caged or IR-caged adenosine triphosphate (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine or other protein of interest selected for the treatment of a cancerous, pre-cancerous, or non-cancerous tumor in the subject. The cytokine or other protein of interest acts in concert with other proteins or cells to enhance a desired immune response for the treatment or reduction in size of the cancerous, pre-cancerous, or non-cancerous tumor. The cytokine or other protein of interest is selected, e.g., to inhibit cell division and/or growth (e.g., a growth factor inhibitor), to inhibit angiogenesis (e.g., an angiogenic factor inhibitor), to promote cell death (e.g., an apoptosis-promoting cytokine or other protein of interest), or to regulate an immune response (e.g., increasing proliferation of cytotoxic T cells, increasing proliferation of helper T cells, maintaining the population of helper T cells, activating cytotoxic T cells, or a combination thereof), in the vicinity of the tumor. Optionally, the method further comprises a step of administering activated T cells to the subject.

Where the nanoliposome comprises a photoactivatable-caged ATP, the plasmid encoding the protein is prepared to express the cytokine or other protein of interest via the cell-free transcription and translation system upon activation of the system in response to exposure to an activating UV light, infrared light (IR) (such as in the presence of upconversion nanoparticles in the microparticle), thereby releasing the UV-caged ATP in order to initiate the processes of transcription and translation of the cytokine or other protein of interest during or after administration of the nanoliposome or microparticle comprising the nanoliposome to a subject in need thereof. Where the tumor is cancerous or pre-cancerous (e.g., a growth comprising cells with at least one pre-cancerous mutation), the cytokine or other protein of interest may be selected based on the type(s) of cells comprising the tumor and, e.g., any cell surface proteins specific to the cancerous or pre-cancerous cells as compared with neighboring healthy tissue.

Where the nanoliposome comprises a photoactivatable-caged ATP, the microparticle comprising the nanoliposome is administered adjacent to the tumor within the subject in need thereof and is exposed to the activating UV or IR light. The UV-caged or IR-caged ATP is released and initiates the processes of transcription and translation of the cytokine or other protein of interest, which is then expressed adjacent to the tumor.

Where the nanoliposome comprises a photoactivatable-caged ATP, and where the tumor is inoperable, it may be possible to use a guided catheter comprising a UV or IR light source to administer the microparticle comprising the nanoliposome adjacent to the tumor.

The regulatable expression of the cytokine or other protein of interest at a focus of interest, namely, specifically treats the localized environment comprising the tumor in the subject.

### Example 8: Treatment of an Autoimmune-Targeted Focus or of a Symptomatic Focus of an Autoimmune Disease with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); optionally, and optionally, an ultraviolet (UV)-caged or an infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and optionally, a UV-caged or IR-caged (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine or other protein of interest selected for the treatment of an autoimmune-targeted focus or symptomatic focus of an autoimmune disease in the subject. If, as a non-limiting example, the subject has rheumatoid arthritis, the cytokine or other protein of interest treatment is administered at or adjacent to joints (e.g., in the hands or feet) particularly inflamed or damaged by the effects of rheumatoid arthritis. If, as a non-limiting example, the subject has psoriasis, the cytokine or other protein of interest treatment is administered at or adjacent to an area of psoriatic rash (e.g., especially if the area is one in which psoriasis is potentially dangerous, such as in close proximity to an eye). If, as a non-limiting example, the subject has eczema, the cytokine or other protein of interest treatment is administered at or adjacent to an area of eczema on the skin. If, as a non-limiting example, the subject has alopecia, the cytokine or other protein of interest treatment is administered at or adjacent to an area of alopecia. If, as a non-limiting example, the subject has vitiligo, the cytokine or other protein of interest treatment is administered at or adjacent to an area of vitiligo. If, as a non-limiting example, the subject has multiple sclerosis, the cytokine or other protein of interest treatment is administered at or adjacent to a damaged myelin sheath.

Alternatively, protein production locally for autoimmune diseases targets the pathogenic antibodies in the disease, for example, a protein that breaks down antibodies in the vicinity (an IgG endopeptidase) or a protein that binds antibodies (a decoy of the antibody's autoimmune target).

The cytokine or other protein of interest acts in concert with other proteins or cells to enhance a desired immune response for the treatment of a localized focus of an autoimmune disease or of one or more localized symptoms of the autoimmune disease. The cytokine or other protein of interest is selected, e.g., to inhibit or promote (as needed) cell division and/or growth (e.g., a growth factor inhibitor), to inhibit inflammation (e.g., anti-inflammatory), to inhibit or promote (as needed) cell death (e.g., an apoptosis-promoting cytokine or other protein of interest), or to regulate an immune response (e.g., decreasing proliferation of cytotoxic T cells, decreasing proliferation of helper T cells, reducing cytotoxic T cells, or a combination thereof, as well as increasing recognition of self), in the vicinity of the autoimmune-targeted or symptomatic focus of the autoimmune disease. Optionally, the method further comprises a step of administering activated T cells to the subject. Alternatively, protein production locally for autoimmune diseases targets the pathogenic antibodies in the disease, for example, a protein that breaks down antibodies in the vicinity (an IgG endopeptidase) or a protein that binds antibodies (a decoy of the antibody's autoimmune target).

Where the nanoliposome comprises a photoactivatable-caged ATP, the plasmid encoding the protein is prepared to express the cytokine or other protein of interest via the cell-free transcription and translation system upon activation of the system in response to exposure to an activating UV light, infrared light (IR) (such as in the presence of upconversion nanoparticles in the microparticle), thereby releasing the UV-caged or IR-caged ATP in order to initiate the processes of transcription and translation of the cytokine or other protein of interest during or after administration of the nanoliposome or microparticle comprising the nanoliposome to a subject in need thereof.

Where the nanoliposome comprises a photoactivatable-caged ATP, the microparticle comprising the nanoliposome is administered at or adjacent the autoimmune-targeted focus or symptomatic focus of the autoimmune disease in the subject and is exposed to the activating UV or IR light. The UV-caged or IR-caged ATP is released and initiates the processes of transcription and translation of the cytokine or other protein of interest, which is then expressed at or adjacent to the autoimmune-targeted focus or symptomatic focus of the autoimmune disease in the subject, thereby treating the disease and/or one or more of its symptoms or effects.

The regulatable expression of the cytokine or other protein of interest at a focus of interest, namely, specifically treats the localized environment comprising the autoimmune-targeted focus or symptomatic focus of the autoimmune disease in the subject.

### Example 9: Treatment of a Reactive Focus of an Allergic Reaction or Hypersensitivity Reaction with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); and optionally an ultraviolet (UV)-caged or an infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and optionally, a UV-caged or IR-caged adenosine triphosphate (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine or other protein of interest selected for the treatment of a reactive focus of an allergic reaction or hypersensitivity reaction in the subject. Non-limiting examples of a reactive focus of an allergic reaction or hypersensitivity reaction in a subject include a skin rash, a hive or hives, or a localized swelling (e.g., from an insect or other bite), as well as other symptoms described elsewhere herein.

The cytokine or other protein of interest acts in concert with other proteins or cells to enhance a desired immune response for the treatment or reduction in one or more symptoms of the localized area of allergic reaction or hypersensitivity reaction. The cytokine or other protein of interest is selected, e.g., to inhibit or promote (as needed) cell division and/or growth (e.g., a growth factor inhibitor), to inhibit inflammation (e.g., anti-inflammatory), to inhibit or promote (as needed) cell death (e.g., an apoptosis-promoting cytokine or other protein of interest), or to regulate an immune response (e.g., decreasing production or accumulation of histamine, increasing proliferation of cytotoxic T cells, increasing proliferation of helper T cells, maintaining the population of helper T cells, activating cytotoxic T cells, or a combination thereof), in the vicinity of the reactive focus of the allergic reaction or hypersensitivity reaction, as well as other activities described elsewhere herein. Optionally, the method further comprises a step of administering activated T cells to the subject.

Where the nanoliposome comprises a photoactivatable-caged ATP, the plasmid encoding the protein is prepared to express the cytokine or other protein of interest via the cell-free transcription and translation system upon activation of the system in response to exposure to an activating UV light, infrared light (IR) (such as in the presence of upconversion nanoparticles in the microparticle), thereby releasing the UV-caged or IR-caged ATP in order to initiate the processes of transcription and translation of the cytokine or other protein of interest during or after administration of the nanoliposome or microparticle comprising the nanoliposome to a subject in need thereof.

Where the nanoliposome comprises a photoactivatable-caged ATP, the microparticle comprising the nanoliposome is administered at or adjacent to the reactive focus of the allergic reaction or hypersensitivity reaction in the subject and is exposed to the activating UV or IR light. The UV-caged or IR-caged ATP is released and initiates the processes of transcription and translation of the cytokine or other protein of interest, which is then expressed at or adjacent to the reactive focus of the allergic reaction or hypersensitivity reaction in the subject, thereby treating the condition and/or one or more of its symptoms or effects.

The regulatable expression of the cytokine or other protein of interest at a focus of interest, namely, specifically treats the localized environment comprising the reactive focus of the allergic reaction or hypersensitivity reaction in the subject in the subject in the subject.

### Example 10: Treatment of a Focus of Infection or Symptoms of a Localized Infection or an Infectious Disease with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); and optionally, an ultraviolet (UV)-caged or an infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and optionally, a UV-caged or IR-caged adenosine triphosphate (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine or other protein of interest selected for the treatment of a focus of infection or symptoms of a localized infection or an infectious disease in the subject. If, as a non-limiting example, the subject has fungal infection (e.g., aspergillus, etc., as described herein above), a bacterial infection (e.g., methicillin-resistant Staphylococcus aureus [MRSA], etc., as described herein above), a viral infection (e.g., a shingles rash from varicella-zoster/herpes zoster; a cold sore/fever blister from, e.g., Herpes simplex I; a genital wart or blister from, e.g., Herpes simplex II, etc., as described herein above), a parasitic infection (e.g., an area infected by scabies, Chagas, Hypoderma tarandi, an amoeba, a roundworm, Toxoplasma gondii, etc., as described herein above), or with respect to other examples described herein, the cytokine or other protein of interest treatment is administered at or adjacent to the infection site, rash, lesion, cold sore, wart, etc., either to treat the infection (e.g., an wound or surgical site infected with MRSA), to contain it or reduce its spread within the subject, to reduce its transmissibility to other individuals (Herpes simplex I or Herpes simplex 11), or to reduce a symptom of the infection at the focus of symptoms (e.g., pain associated with an outbreak of shingles).

Alternatively, proteins could be deployed as decoys of pathogenic proteins made by the pathogen. For example, a protease made by the pathogen could be targeted by a decoy substrate here.

The cytokine or other protein of interest acts in concert with other proteins or cells to enhance a desired immune response for the treatment of a localized focus of an infection or infectious disease or of one or more localized symptoms of the infection or infectious disease. The cytokine or other protein of interest is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the focus of infection or symptoms of a localized infection or an infectious disease. Optionally, the method further comprises a step of administering activated T cells to the subject.

Where the nanoliposome comprises a photoactivatable-caged ATP, the plasmid encoding the protein is prepared to express the cytokine or other protein of interest via the cell-free transcription and translation system upon activation of the system in response to exposure to an activating UV light, infrared light (IR) (such as in the presence of upconversion nanoparticles in the microparticle), thereby releasing the UV-caged or IR-caged ATP in order to initiate the processes of transcription and translation of the cytokine or other protein of interest during or after administration of the nanoliposome or microparticle comprising the nanoliposome to a subject in need thereof.

Where the nanoliposome comprises a photoactivatable-caged ATP, the microparticle comprising the nanoliposome is administered at or adjacent the focus of infection or symptoms of the localized infection or infectious disease in the subject and is exposed to the activating UV or IR light. The UV-caged or IR-caged ATP is released and initiates the processes of transcription and translation of the cytokine or other protein of interest, which is then expressed at or adjacent to the focus of infection and/or the focus of symptoms of the localized infection or infectious disease in the subject, thereby treating the disease and/or one or more of its symptoms or effects. It is to be noted that the focus of infection (e.g., inactive site of varicella-zoster/herpes zoster virus in nerve cells) may not be identical to the site of the focus of symptoms (e.g., shingles rash).

The regulatable expression of the cytokine or other protein of interest at a focus of interest, namely, specifically treats the localized environment comprising focus of infection or symptoms of the localized infection or infectious disease in the subject.

### Example 11: Treatment of an Injury or a Site of Chronic Damage with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); and optionally, an ultraviolet (UV)-caged or an infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and optionally, a UV-caged or IR-caged adenosine triphosphate (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine or other protein of interest selected for the treatment of an injury (e.g., trauma, chemical, etc., as described herein above) or of a site of chronic damage (e.g., osteoarthritis, vitiligo, etc., as described herein above) in the subject. The cytokine or other protein of interest treatment is administered at or adjacent to the injury or to the site of chronic damage, either to treat, reduce, or alleviate the injury (e.g., to promote repair, to promote vascularization, to regrow cells, to reduce scarring, etc., as described herein above), to prevent infection or further damage (e.g., fungal, bacterial, viral, or parasitic infection; neuropathy; muscle wasting; inflammation, scarring, or obstruction due to blockage of the luminal space, etc., as described herein above), or to reduce a symptom of the injury or of the chronic damage (e.g., pain, inflammation, swelling, fever, etc., as described herein above).

The cytokine or other protein of interest acts in concert with other proteins or cells to enhance a desired immune response for the treatment of an injury or a site of chronic damage or of one or more localized symptoms of the injury or the chronic damage. The cytokine or other protein of interest is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the injury or the site of chronic damage. Optionally, the method further comprises a step of administering activated T cells to the subject.

Where the nanoliposome comprises a photoactivatable-caged ATP, the plasmid encoding the protein is prepared to express the cytokine or other protein of interest via the cell-free transcription and translation system upon activation of the system in response to exposure to an activating UV light, infrared light (IR) (such as in the presence of upconversion nanoparticles in the microparticle), thereby releasing the UV-caged or IR-caged ATP in order to initiate the processes of transcription and translation of the cytokine or other protein of interest during or after administration of the nanoliposome or microparticle comprising the nanoliposome to a subject in need thereof.

Where the nanoliposome comprises a photoactivatable-caged ATP, the microparticle comprising the nanoliposome is administered at or adjacent to the injury or to the site of chronic damage in the subject and is exposed to the activating UV or IR light. The UV-caged or IR-caged ATP is released and initiates the processes of transcription and translation of the cytokine or other protein of interest, which is then expressed at or adjacent to the injury or to the site of chronic damage in the subject, thereby treating the disease and/or one or more of its symptoms or effects.

The regulatable expression of the cytokine or other protein of interest at a focus of interest, namely, specifically treats the localized environment comprising the injury or the site of chronic infection in the subject.

### Example 12: Treatment of a Surgical Site with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); and optionally, an ultraviolet (UV)-caged or an infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and optionally, a UV-caged or IR-caged adenosine triphosphate (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine or other protein of interest selected for the treatment of a surgical site in the subject. The cytokine or other protein of interest treatment is administered at or adjacent to the surgical site, either to treat, reduce, or alleviate the effects of surgery (e.g., to promote repair, to promote vascularization, etc., as described herein above), to prevent infection or further damage (e.g., fungal, bacterial, viral, or parasitic infection; neuropathy; muscle wasting; etc., as described herein above), or to reduce a symptom of the effects of surgery (e.g., pain, inflammation, etc., as described herein above).

The cytokine or other protein of interest acts in concert with other proteins or cells to enhance a desired immune response for the treatment of a surgical site or of one or more localized symptoms of the associated effects of surgery. The cytokine or other protein of interest is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the surgical site. Optionally, the method further comprises a step of administering activated T cells to the subject.

Where the nanoliposome comprises a photoactivatable-caged ATP, the plasmid encoding the protein is prepared to express the cytokine or other protein of interest via the cell-free transcription and translation system upon activation of the system in response to exposure to an activating UV light, infrared light (IR) (such as in the presence of upconversion nanoparticles in the microparticle), thereby releasing the UV-caged or IR-caged ATP in order to initiate the processes of transcription and translation of the cytokine or other protein of interest during or after administration of the nanoliposome or microparticle comprising the nanoliposome to a subject in need thereof.

Where the nanoliposome comprises a photoactivatable-caged ATP, the microparticle comprising the nanoliposome is administered at or adjacent to the surgical site in the subject and is exposed to the activating UV or IR light. The UV-caged or IR-caged ATP is released and initiates the processes of transcription and translation of the cytokine or other protein of interest, which is then expressed at or adjacent to the surgical site in the subject, thereby treating the disease and/or one or more of the symptoms or effects of surgery.

The regulatable expression of the cytokine or other protein of interest at a focus of interest, namely, specifically treats the localized environment comprising the surgical site in the subject.

### Example 13: Treatment of a Transplant Site Associated with a Transplanted Organ, Tissue, or Cells with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); and optionally, an ultraviolet (UV)-caged or an infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and optionally, a UV-caged or IR-caged adenosine triphosphate (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine or other protein of interest selected for the treatment of a transplant site associated with a transplanted organ, tissue, or cells in the subject. The cytokine or other protein of interest treatment is administered at or adjacent to the transplant site associated with a transplanted organ, tissue, or cells, either to treat, reduce, or alleviate the surgery related to the transplant (e.g., to promote repair, to promote vascularization, etc., as described herein above), to prevent infection or damage (e.g., fungal, bacterial, viral, or parasitic infection; neuropathy; muscle wasting; etc., as described herein above), to reduce the likelihood of rejection, or to reduce a symptom of the transplant or surgery related thereto (e.g., pain, inflammation, etc., as described herein above).

The cytokine or other protein of interest acts in concert with other proteins or cells to enhance a desired immune response for the treatment of the transplant site associated with a transplanted organ, tissue, or cells. The cytokine or other protein of interest is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response, such as suppression of rejection (e.g., decreasing proliferation of cytotoxic T cells, decreasing proliferation of helper T cells, decreasing cytotoxic T cells, or a combination thereof), in the vicinity of the injury or the site of chronic damage.

Where a transplant site is the focus of interest, optionally, activated regulatory T cells may be added.

Where the nanoliposome comprises a photoactivatable-caged ATP, the plasmid encoding the protein is prepared to express the cytokine or other protein of interest via the cell-free transcription and translation system upon activation of the system in response to exposure to an activating UV light, infrared light (IR) (such as in the presence of upconversion nanoparticles in the microparticle), thereby releasing the UV-caged ATP in order to initiate the processes of transcription and translation of the cytokine or other protein of interest during or after administration of the nanoliposome or microparticle comprising the nanoliposome to a subject in need thereof.

Where the nanoliposome comprises a photoactivatable-caged ATP, the microparticle comprising the nanoliposome is administered at or adjacent to the transplant site associated with a transplanted organ, tissue, or cells in the subject and is exposed to the activating UV or IR light. The UV-caged or IR-caged ATP is released and initiates the processes of transcription and translation of the cytokine or other protein of interest, which is then expressed at or adjacent to the transplant site associated with a transplanted organ, tissue, or cells in the subject, thereby reducing the likelihood of rejection and/or one or more of its symptoms or effects, as well as the symptoms or effects of the transplant surgery.

The regulatable expression of the cytokine or other protein of interest at a focus of interest, namely, specifically treats the localized environment comprising the transplant site associated with a transplanted organ, tissue, or cells in the subject.

### Example 14: Treatment of a Blood Clot Causing or at Risk for Causing a Myocardial Infarction, an Ischemic Stroke, or a Pulmonary Embolism with Protein-Expressing Nanoliposomes and Microparticles

One or more nanoliposomes are produced comprising a cell-free transcription and translation system; a plasmid comprising a nucleic acid encoding a cytokine or other protein of interest (e.g., a chemokine, a therapeutic antibody or fragment thereof, an antigen-binding protein, a Fc fusion protein, an anticoagulant, an enzyme, a hormone, or a thrombolytic); and optionally, an ultraviolet (UV)-caged or an infrared (IR)-caged adenosine triphosphate (ATP), as described in Examples 5 and 6.

The nanoliposome is optionally contained in a microparticle. One or more microparticles are produced comprising at least one nanoliposome, the nanoliposome comprising a cell-free transcription and translation system; a plasmid comprising a nucleic acid encoding a protein of interest; and a UV-caged or IR-caged adenosine triphosphate (ATP), as described in Examples 5 and 6.

The microparticle optionally comprises alginate or alginate-heparin, either as a component or as a coating. The microparticle optionally further comprises a lipid membrane coating, optionally comprising POPC. The microparticle optionally comprises one or more supermagnetic iron oxide nanoparticles (SPION) and/or one or more upconversion nanoparticles, as described in Examples 5 and 6.

The protein is a cytokine, thrombolytic or other protein of interest selected for the treatment of a blood clot causing or at risk for causing a myocardial infarction, an ischemic stroke, or a pulmonary embolism in the subject. In a non-limiting example, thrombolytic (“clot buster”) treatment is administered at or adjacent to the blood clot to break up, reduce, or eliminate the blood clot in order to treat or prevent infarction of a blood vessel and thereby to treat or prevent, e.g., a myocardial infarction (heart attack), an ischemic stroke, or a pulmonary embolism. Non-limiting examples of thrombolytics include tissue plasminogen activator (tPA), tenecteplase, alteplase, urokinase, reteplase, and streptokinase.

It is noted that the location of the blood clot may not be in the heart, the brain, or a lung at the time of treatment, but rather in some other part of the subject's body (e.g., the lower limbs and extremities; the carotid artery; the site of an injury, surgery, or a transplant; or elsewhere).

The cytokine, thrombolytic, or other protein of interest acts in concert with other proteins or cells to enhance a desired response for the treatment of a blood clot. A non-limiting example of a desired response includes blocking tissue factors that promote clotting (e.g., blocking factor III or factor XII). The cytokine, thrombolytic or other protein of interest is selected, e.g., to inhibit angiogenesis, to promote reduction or elimination of clotting, to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the blood clot. Optionally, the method further comprises a step of administering activated T cells to the subject.

Where the nanoliposome comprises a photoactivatable-caged ATP, the plasmid encoding the protein is prepared to express the cytokine, thrombolytic, or other protein of interest via the cell-free transcription and translation system upon activation of the system in response to exposure to an activating UV light, infrared light (IR) (such as in the presence of upconversion nanoparticles in the microparticle), thereby releasing the UV-caged ATP in order to initiate the processes of transcription and translation of the cytokine, thrombolytic, or other protein of interest during or after administration of the nanoliposome or microparticle comprising the nanoliposome to a subject in need thereof.

Where the nanoliposome comprises a photoactivatable-caged ATP, the microparticle comprising the nanoliposome is administered at or adjacent to the blood clot in the subject and is exposed to the activating UV or IR light. The UV-caged or IR-caged ATP is released and initiates the processes of transcription and translation of the cytokine, thrombolytic, or other protein of interest, which is then expressed at or adjacent to the blood clot in the subject, thereby reducing or eliminating the blood clot and/or one or more of its symptoms or effects.

The composition may be administered via a guided catheter with a UV or IR light, which may facilitate access to, and treatment of, the blood clot. The composition may be administered, in a non-limiting example, together with angioplasty (e.g., a balloon catheter) or other clot removal treatment.

The regulatable expression of the cytokine, thrombolytic, or other protein of interest at a focus of interest, namely, specifically treats the localized environment comprising the blood clot in the subject.

### Materials and Methods for Examples 15-22

The following materials and methods were used in the Examples below. Where appropriate, the Examples also site relevant literature disclosing the same or similar methods.

### Chemicals and Biologicals:

Unless noted otherwise, all chemicals were purchased from SIGMA-ALDRICH™, INC. (St. Louis, Mo.). All the cell culture reagents, solutions, and dishes were obtained from THERMO FISHER SCIENTIFIC™ (Waltham, Mass.) except as indicated otherwise. Interleukin-2 (IL-2) used for this study was provided by the BRB Preclinical Repository of the National Cancer Institute, Frederick, Md., USA.

### Plasmid DNA

### Plasmid DNA Preparation and Caging

Interleukin-2/green fluorescent protein (IL-2-GFP) (ADDGENE™ MA, USA, cat. #67053, 5280 bp) plasmid DNA was selected as a plasmid of interest, an expression vector comprising DNA encoding IL-2 operably linked to DNA encoding green fluorescent protein. In order to add spatiotemporal control on encoding process, the plasmid DNA was caged using an ultraviolet-sensitive cleavable molecule. The “caged ATP” cage compound (1-(4,5-dimethoxy-2-nitrophenyl) ethyl ester (DMNPE) was used, but a skilled artisan would recognize that other cage compounds could be utilized. Activation of DMNPE and caging procedure followed methods known in the art (Ghosn, B., Haselton, F. R., Gee, K. R. & Monroe, W. T. Control of DNA Hybridization with Photocleavable Adducts¶. Photochemistry and photobiology 81, 953-959 (2005); Schroeder, A. et al. Remotely activated protein-producing nanoparticles. Nano letters 12, 2685-2689 (2012)). Briefly, 1,2 DMNPE (10 mg) and manganese (IV) oxide (MnO2 (MnO2); 50 mg) were gently mixed in dimethyl formamide (1.5 mL) for 30 min at room temperature. MnO2 was removed from the DMNPE through centrifugation followed by filtering. Purified and activated DMNPE was mixed with plasmid DNA (0.5 mg DNA per mL of DMNPE) in Tris HCl buffer (10 mM, pH 5.5) at 4° C. for 24 h. Excess DMNPE was removed using AMICON ULTRA™-0.5 (Ultracel™-3 membrane, MWCO 3 kDa). Caged plasmids were ultra-centrifugated to adjust concentration and stored in the dark at 4° C. before use. Two other plasmid DNAs, V72 ELP (ADDGENE™ MA, USA, cats. #68938, 6565 bp) (Dhandhukia et al. (2013) Biomacromolecules 14(4): 976-985) and Renilla luciferase (PROMEGA™, 3320 bp), were also used to test the effect of the molecular weight of encapsulation and expression efficiencies.

### Super2 (IL-2 Superkine) Construct Design

To construct the H9 IL-2 superkine expression system, the pCellFree_G03_IL2 expression plasmid (ADDGENE™ #67053; www.addgene.org/67053/) (Gagoski et al. (2015) Biotechnol. Bioeng. doi: 10.1002/bit.25814) was modified using NEBuilder® HiFi DNA Assembly. H9 IL-2 superkine cDNA (ADDGENE™ #41808; www.addgene.org/41808/) (Levin et al. (2012) Nature 484 (7395): 529-533 doi: 10.1038/nature10975) was amplified using forward 5′-TTATTTTATTTTATTTAACCGGAGCCATGGGAGAATTCGC-3′ (SEQ ID NO: 1) and reverse 5′-GGAGGAGGGCGGCCGCTTACCTAAGTTAGTGTTGAGATGATGC-3′ (SEQ ID NO: 2) primers, with the primers including 20 base pair overlaps (italicized and underlined) for insertion into the pCellFree backbone. The pCellFree_G03_IL2 backbone was amplified in two-parts, with overlaps included for insertion of the IL-2 amplification and overlaps generated in the AmpR gene to serve as a positive marker for correct HiFi DNA assembly. The first section of the backbone was amplified using forward 5′-GCGAATTCTCCCATGGCTCCGGTTAAATAAAATAAAATAA-3′ (SEQ ID NO: 3) and reverse 5′-TTGCTACAGGCATCGTGGTGTCACGCTCGTCGTTTGGTAT-3′ (SEQ ID NO: 4) primers. The second section of the backbone was amplified using forward 5′-ATACCAAACGACGAGCGTGACACCACGATGCCTGTAGCAA-3′ (SEQ ID NO: 5) and reverse 5′-GCATCATCTCAACACTAACTTAGGTAAGCGGCCGCCCTCCTCC-3′ (SEQ ID NO: 6). The amplification and assembly strategy excluded the GFP gene present on the original pCellFree_G03_H9 plasmid. Once all three constructs were successfully PCR amplified, they were assembled using NEBuilder® HiFi DNA Assembly Master Mix (cat. #E2621).

### Super2 (IL-2 Superkine) Construct Sequence

The Super2 (IL-2 superkine) Construct Sequence is shown in TABLE 1.

TABLE 1
Super2 Sequence and Plasmid Sequence.

| | Sequence |
|---|---|
| Sample | (SEQ ID NO) |
| IL-2 Superkine (Super2) | GGAGCCATGGGAGAATTCGCACCTACTTCAAGTT |
| nucleotide sequence | CTACAAAGAAAACACAGCTACAACTGGAGCATT |
| | TACTTCTGGATTTACAGATGATTTTGAATGGAAT |
| | TAATAATTACAAGAATCCCAAACTCACCAGGAT |
| | GCTCACATTTAAGTTTTACATGCCCAAGAAGGCC |
| | ACAGAACTGAAACATCTTCAGTGTCTAGAAGAA |
| | GAACTCAAACCTCTGGAGGAAGTGCTAAATTTA |
| | GCTCAGAGCAAAAACTTTCACTTCGATCCCAGGG |
| | ACGTCGTCAGCAATATCAACGTATTCGTCCTGGA |
| | ACTAAAGGGATCTGAAACAACATTCATGTGTGA |
| | ATATGCTGATGAGACAGCAACCATTGTAGAATTT |
| | CTGAACAGATGGATTACCTTTTGTCAAAGCATCA |
| | TCTCAACACTAACTCAT (SEQ ID NO: 7) |
| pCellFree_G03_H9 | CCCGAAAAGTGCCACCTGACGTCTAATACGACTC |
| (IL-2 Superkine | ACTATAGGGACATCTTAAGTTTATTTTATTTTATT |
| [Super2] Plasmid) | TTATTTAACCGGAGCCATGGGAGAATTCGCACCT |
| | ACTTCAAGTTCTACAAAGAAAACACAGCTACAA |
| | CTGGAGCATTTACTTCTGGATTTACAGATGATTT |
| | TGAATGGAATTAATAATTACAAGAATCCCAAACT |
| | CACCAGGATGCTCACATTTAAGTTTTACATGCCC |
| | AAGAAGGCCACAGAACTGAAACATCTTCAGTGT |
| | CTAGAAGAAGAACTCAAACCTCTGGAGGAAGTG |
| | CTAAATTTAGCTCAGAGCAAAAACTTTCACTTCG |
| | ATCCCAGGGACGTCGTCAGCAATATCAACGTATT |
| | CGTCCTGGAACTAAAGGGATCTGAAACAACATT |
| | CATGTGTGAATATGCTGATGAGACAGCAACCATT |
| | GTAGAATTTCTGAACAGATGGATTACCTTTTGTC |
| | AAAGCATCATCTCAACACTAACTCATTAGGTAAG |
| | CGGCCGCCCTCCTCCTCCTTTCTTGTTCCTTTCAC |
| | GTCGCCTTCTCGGTTGTAGCTGGCAGACGACGAG |
| | TCTTACTTTTACGTGTACTTCTCTATAGATGATGT |
| | ATGATCTCTCTGCATGCGTGTTCGTGCATGTGTC |
| | CGTGTGTTGGGTACGCGTGGTACCCTGCAGGAAG |
| | GAAGCTGAGTTGGCTGCTGCCACCGCTGAGCAAT |
| | AACTAGTAATTACTAGCATAACCCCTTGGGGCCT |
| | CTAAACGGGTCTTGAGGGGGTTTTTTGCTGAAAG |
| | GAGGACAGCTGATGATTGTCATGCTTGCCATCTG |
| | TTTTCTTGCAAGGTCAGAGGAATTCGTAATCATG |
| | GTCATAGCTGTTTCCTGTGTGAAATTGTTATCCG |
| | CTCACAATTCCACACAACATACGAGCCGGAAGC |
| | ATAAAGTGTAAAGCCTGGGGTGCCTAATGAGTG |
| | AGCTAACTCACATTAATTGCGTTGCGCTCACTGC |
| | CCGCTTTCCAGTCGGGAAACCTGTCGTGCCAGCT |
| | GCATTAATGAATCGGCCAACGCGCGGGGAGAGG |
| | CGGTTTGCGTATTGGGCGCTCTTCCGCTTCCTCGC |
| | TCACTGACTCGCTGCGCTCGGTCGTTCGGCTGCG |
| | GCGAGCGGTATCAGCTCACTCAAAGGCGGTAAT |
| | ACGGTTATCCACAGAATCAGGGGATAACGCAGG |
| | AAAGAACATGTGAGCAAAAGGCCAGCAAAAGGC |
| | CAGGAACCGTAAAAAGGCCGCGTTGCTGGCGTT |
| | TTTCCATAGGCTCCGCCCCCCTGACGAGCATCAC |
| | AAAAATCGACGCTCAAGTCAGAGGTGGCGAAAC |
| | CCGACAGGACTATAAAGATACCAGGCGTTTCCCC |
| | CTGGAAGCTCCCTCGTGCGCTCTCCTGTTCCGAC |
| | CCTGCCGCTTACCGGATACCTGTCCGCCTTTCTC |
| | CCTTCGGGAAGCGTGGCGCTTTCTCATAGCTCAC |
| | GCTGTAGGTATCTCAGTTCGGTGTAGGTCGTTCG |
| | CTCCAAGCTGGGCTGTGTGCACGAACCCCCCGTT |
| | CAGCCCGACCGCTGCGCCTTATCCGGTAACTATC |
| | GTCTTGAGTCCAACCCGGTAAGACACGACTTATC |
| | GCCACTGGCAGCAGCCACTGGTAACAGGATTAG |
| | CAGAGCGAGGTATGTAGGCGGTGCTACAGAGTT |
| | CTTGAAGTGGTGGCCTAACTACGGCTACACTAGA |
| | AGAACAGTATTTGGTATCTGCGCTCTGCTGAAGC |
| | CAGTTACCTTCGGAAAAAGAGTTGGTAGCTCTTG |
| | ATCCGGCAAACAAACCACCGCTGGTAGCGGTGG |
| | TTTTTTTGTTTGCAAGCAGCAGATTACGCGCAGA |
| | AAAAAAGGATCTCAAGAGGATCCTTTGATCTTTT |
| | CTACGGGGTCTGACGCTCAGTGGAACGAAAACT |
| | CACGTTAAGGGATTTTGGTCATGAGATTATCAAA |
| | AAGGATCTTCACCTAGATCCTTTTAAATTAAAAA |
| | TGAAGTTTTAAATCAATCTAAAGTATATATGAGT |
| | AAACTTGGTCTGACAGTTACCAATGCTTAATCAG |
| | TGAGGCACCTATCTCAGCGATCTGTCTAGTTCGT |
| | TCATCCATAGTTGCCTGACTCCCCGTCGTGTAGA |
| | TAACTACGATACGGGAGGGCTTACCATCTGGCCC |
| | CAGTGCTGCAATGATACCGCGAGACCCACGCTC |
| | ACCGGCTCCAGATTTATCAGCAATAAACCAGCCA |
| | GCCGGAAGGGCCGAGCGCAGAAGTGGTCCTGCA |
| | ACTTTATCCGCCTCCATCCAGTCTATTAATTGTTG |
| | CCGGGAAGCTAGAGTAAGTAGTTCGCCAGTTAA |
| | TAGTTTGCGCAACGTTGTTGCCATTGCTACAGGC |
| | ATCGTGGTGTCACGCTCGTCGTTTGGTATGGCTT |
| | CATTCAGCTCCGGTTCCCAACGATCAAGGCGAGT |
| | TACATGATCCCCCATGTTGTGCAAAAAAGCGGTT |
| | AGCTCCTTCGGTCCTCCGATCGTTGTCAGAAGTA |
| | AGTTGGCCGCAGTGTTATCACTCATGGTTATGGC |
| | AGCACTGCATAATTCTCTTACTGTCATGCCATCC |
| | GTAAGATGCTTTTCTGTGACTGGTGAGTACTCAA |
| | CCAAGTCATTCTGAGAATAGTGTATGCGGCGACC |
| | GAGTTGCTCTTGCCCGGCGTCAATACGGGATAAT |
| | ACCGCGCCACATAGCAGAACTTTAAAAGTGCTC |
| | ATCATTGGAAAACGTTCTTCGGGGCGAAAACTCT |
| | CAAGGATCTTACCGCTGTTGAGATCCAGTTCGAT |
| | GTAACCCACTCGTGCACCCAACTGATCTTCAGCA |
| | TCTTTTACTTTCACCAGCGTTTCTGGGTGAGCAA |
| | AAACAGGAAGGCAAAATGCCGCAAAAAAGGGA |
| | ATAAGGGCGACACGGAAATGTTGAATACTCATA |
| | CTCTTCCTTTTTCAATATTATTGAAGCATTTATCA |
| | GGGTTATTGTCTCATGAGCGGATACATATTTGAA |
| | TGTATTTAGAAAAATAAACAAATAGGGGTTCCG |
| | CGCACATTTC (SEQ ID NO: 9) |
| IL-2 Superkine (Super2) | MGEFAPTSSSTKKTQLQLEHLLLDLQMILNGINNY |
| | KNPKLTRMLTFKFYMPKKATELKHLQCLEEELKPL |
| | EEVLNLAQSKNFHFDPRDVVSNINVFVLELKGSETT |
| | FMCEYADETATIVEFLNRWITFCQSIISTLTH (SEQ |
| | ID NO: 10) |

### Nanoliposome Formation

### Bulk Method

Liposomes were formed according to procedures reported previously (Jahn, A., Vreeland, W. N., DeVoe, D. L., Locascio, L. E. & Gaitan, M. Microfluidic directed formation of liposomes of controlled size. Langmuir 23, 6289-6293 (2007)), with some modification. Briefly, 1,2-dimyristoyl-sn-glycero-3-phosphocholine (DMPC), 1-palmitoyl-2-oleoyl-phosphatidylcholine (POPC), and cholesterol (all from AVANTI POLAR LIPIDS™) in a molar ratio of 6:2:4 were dissolved in dry chloroform and the solvent was evaporated at room temperature under a nitrogen flow to form a dry lipid cake. Afterwards, the cake was further dried in vacuum oven overnight (mixture #1).

The composition of this mixture can be varied. In some embodiments, DMPC is from about 20 molar percent to about 60 molar percent; POPC is from about 20 molar percent to about 40 molar percent; and cholesterol is from about 0 molar percent to about 60 molar percent.

To form nanoliposomes, caged-ATP and DNA were mixed with the cell extract. S30 T7 HIGH-YIELD™ Protein Expression System (PROMEGA™) was used as E. coli extract source. DNA containing solution was added to warmed mixture #1 and vortexed briefly. The non-encapsulated extracts and DNA were separated from formed liposomes by centrifugation at 10,000 rpm for 20 min and washed at 4° C. with 5% dextrose twice, followed by resuspension in either deionized water, phosphate buffered saline (PBS), or cell culture medium. After 1 h of incubation under gentle shaking at 37° C., the nanoliposome formed via extrusion technique of solution by 800, 400, 200, 100 nm pores track-etched polycarbonate membranes (WHATMAN® NUCLEOPORE™ Hydrophilic Membranes) at 37° C.

### Microfluidic Fabrication Method

Microfluidic devices were fabricated with poly(dimethylsiloxane) (PDMS) using a standard micro-molding process (Qin, D., Xia, Y. & Whitesides, G. M. Soft lithography for micro- and nanoscale patterning. Nature Protocols 5, 491 (2010)). To make the master molds, silicon wafers were coated with 60 microns (μm) thick SU-8 photocurable epoxy. Baking, lithography, and development procedures were performed according to previously published reports (Majedi, F. S. et al. On-chip fabrication of paclitaxel-loaded chitosan nanoparticles for cancer therapeutics. Advanced Functional Materials 24, 432-441 (2014)) to obtain negative microchannels on the wafer. The wafers were then annealed and coated with trimethylethoxysilane to prevent PDMS from sticking to the mold. PDMS (SYLGRAD™ 184) oligomer and curing agent were mixed (10:1 ratio), cast on the mold, degassed in desiccators, and cured in an oven at 60° C. for 2 h. PDMS was removed from the mold and in-/outlet holes were punched. Oxygen plasma at 100 mW for 1 min was used to bound PDMS to a glass slide. The mixing channel was 120 microns (μm) wide, 60 microns (μm) high and 15 mm long.

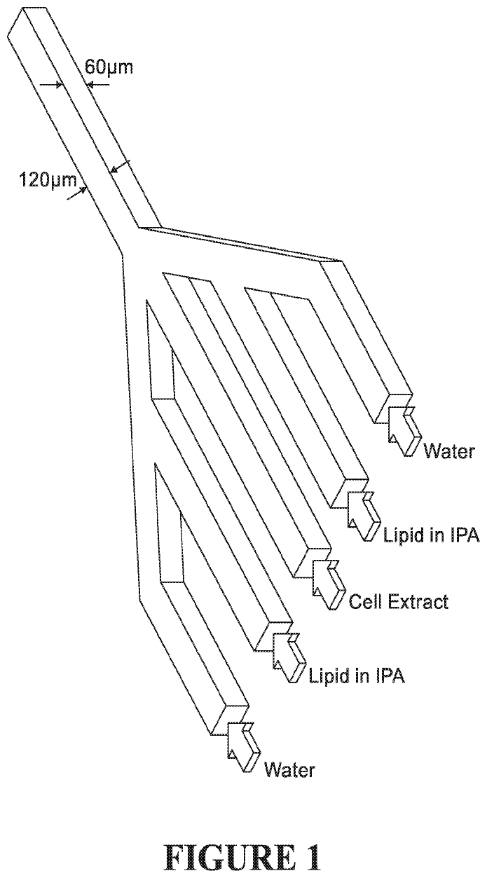

To form nanoliposomes using microfluidic technique, the mixture #1 was dissolved in dry isopropyl alcohol (WA) at a 5 mg/mL. The lipid-IPA was injected into the two channels flanking the center channel (FIG. 1). DMNPE-caged DNA was mixed with the E. coli transcription/translation extract and was used as source and was injected into the center channel of the microfluidic platform. Phosphate buffered saline (PBS) was injected into two lateral channels. Alternatively, water may be used (FIG. 1) or another suitable buffer. The flow rate ratio (FR) was defined as volumetric flow rate of lipid-IPA solution to the overall (lipid-WA+cell extract+PBS) volumetric flow rate.

Alternatively, a different volatile solvent may be used. Examples of volatile solvents for microfluidic protein formation include, but are not limited to acetone, chloroform, isopropanol, and methanol.

Nanoliposome formation at different flow conditions was studied by tuning the flow rates of the PBS streams. The flow rates for samples prepared via microfluidics (“μF”-μF-1, μF-2, μF-3, and μF-4) are summarized in TABLE 2, and details of the particle formulations used in this study are summarized in TABLE 3. Bulk-1 and Bulk-2 were prepared via the Bulk method (TABLE 3).

TABLE 2
Flow Conditions Used.

| Volumetric Flow Rate | | | | | |
|---|---|---|---|---|---|
| (microliters/min [μL/min]) | | | | | Flow Rate |
| | Extract | Lipid-IPA | PBS | Total | Ratio (FR) |
| μF-1 | 2 | 5 | 55 | 62 | 0.08 |
| μF-2 | 2 | 5 | 45 | 52 | 0.10 |
| μF-3 | 2 | 5 | 30 | 37 | 0.14 |
| μF-4 | 2 | 5 | 15 | 22 | 0.23 |

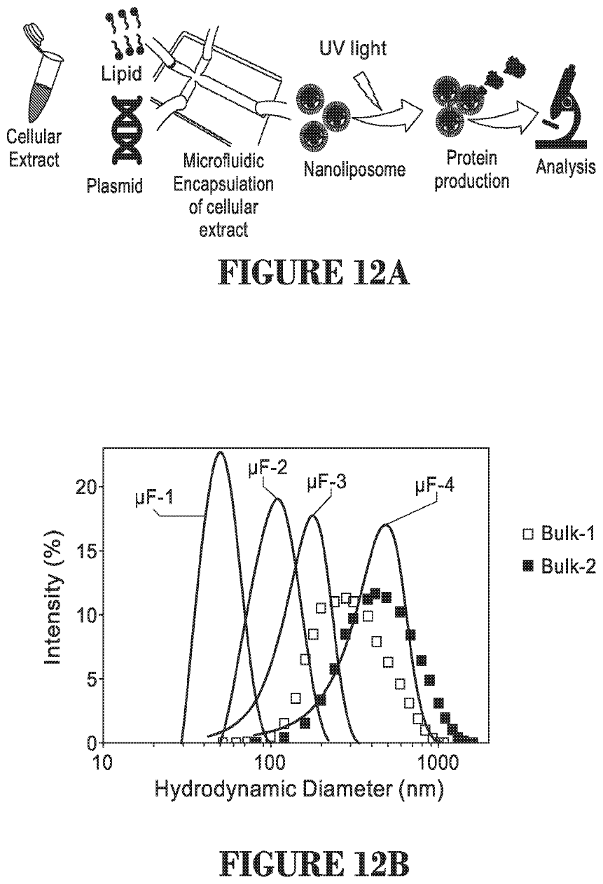

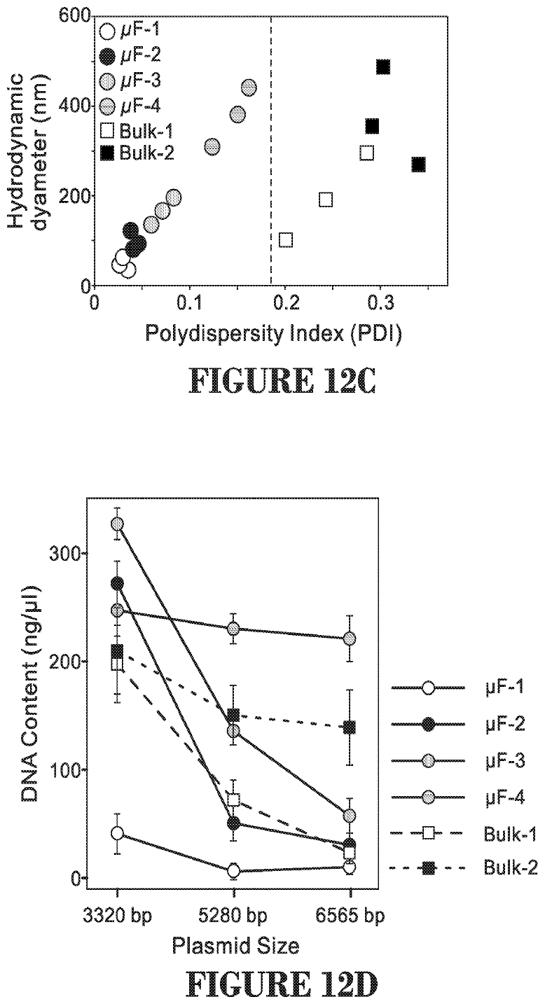

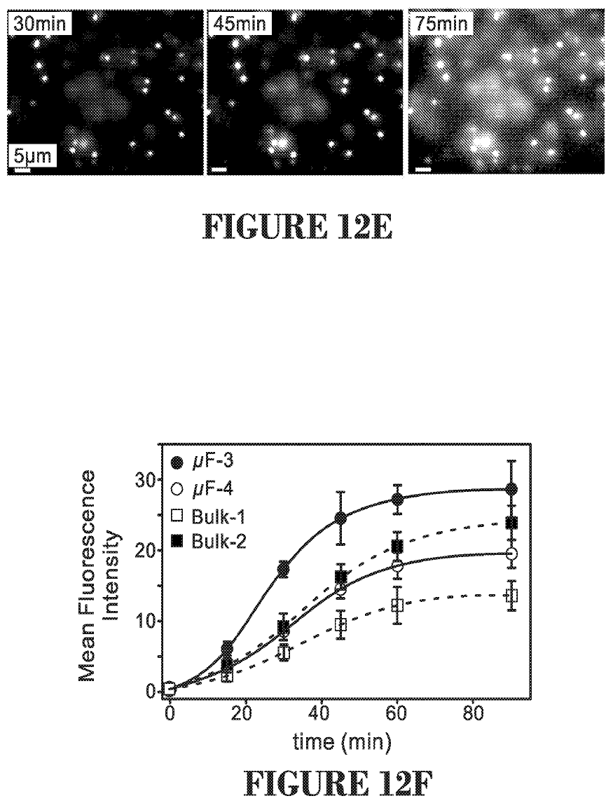

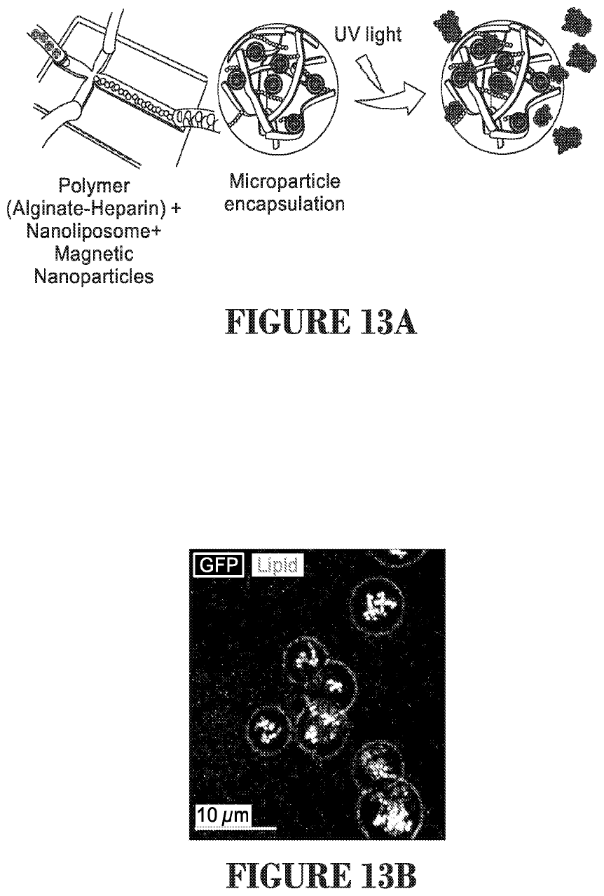

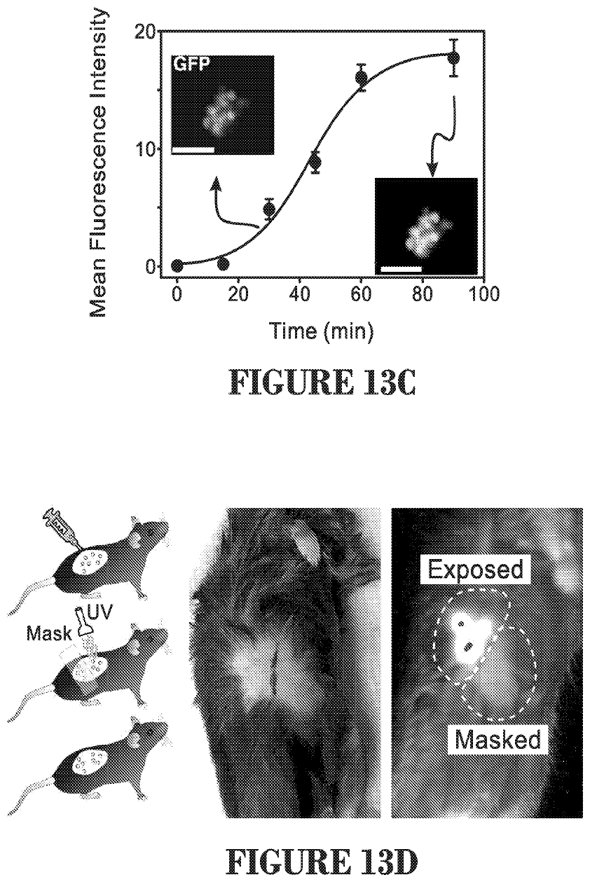

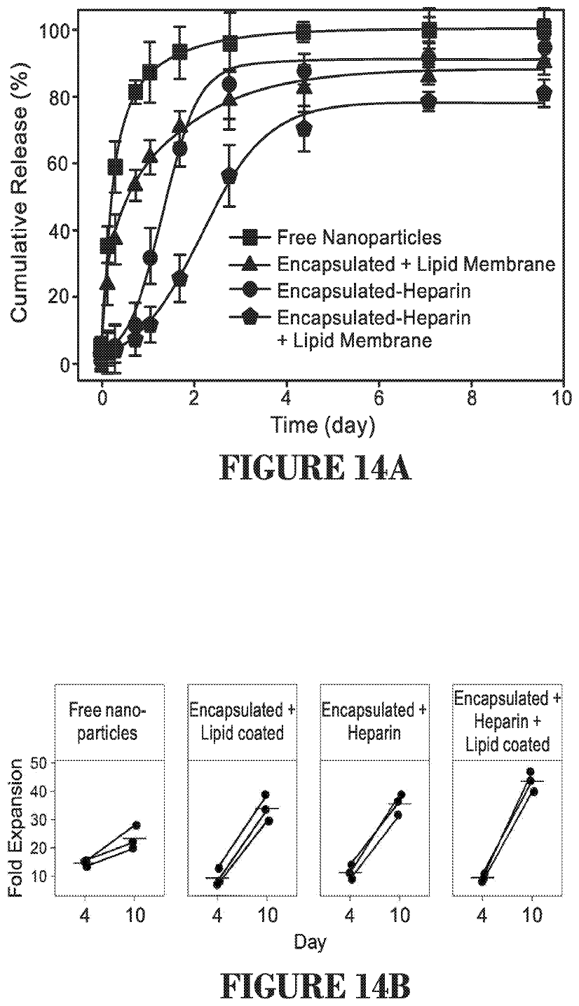

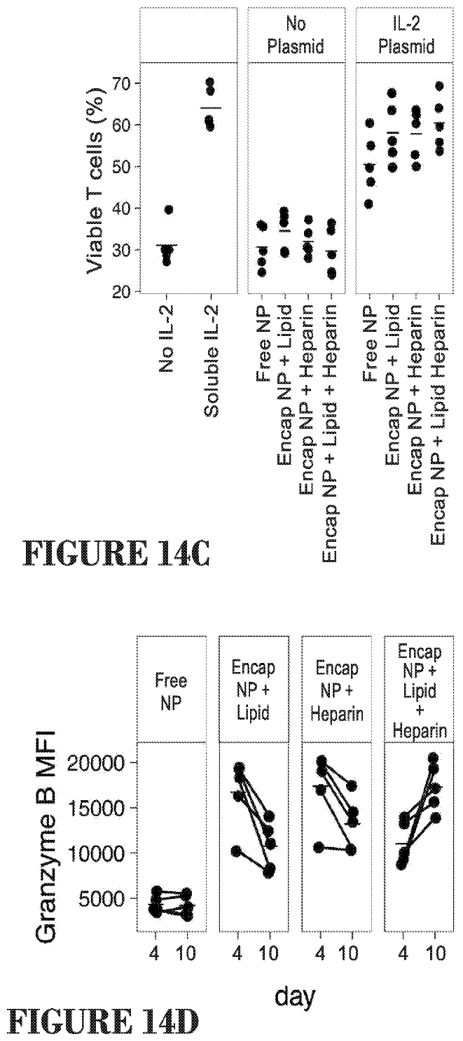

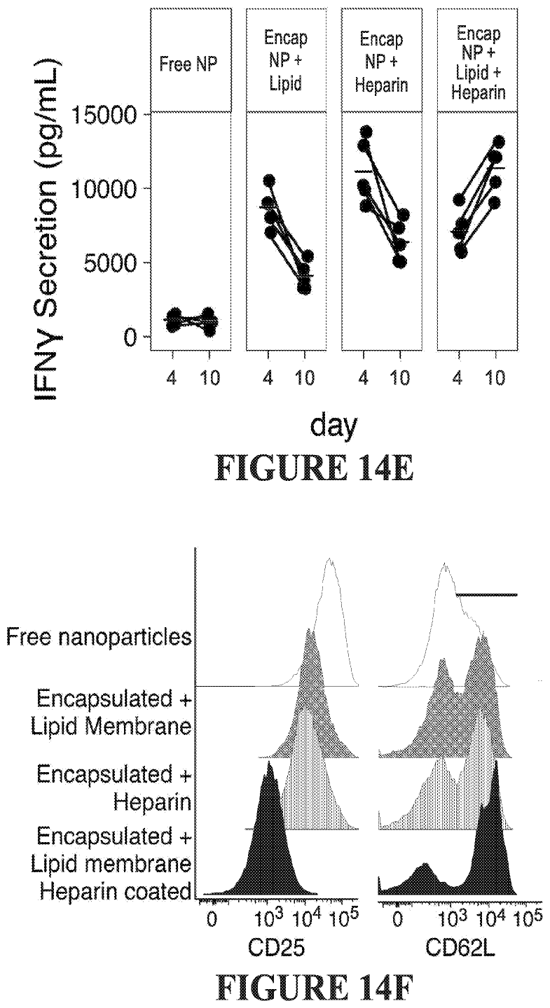

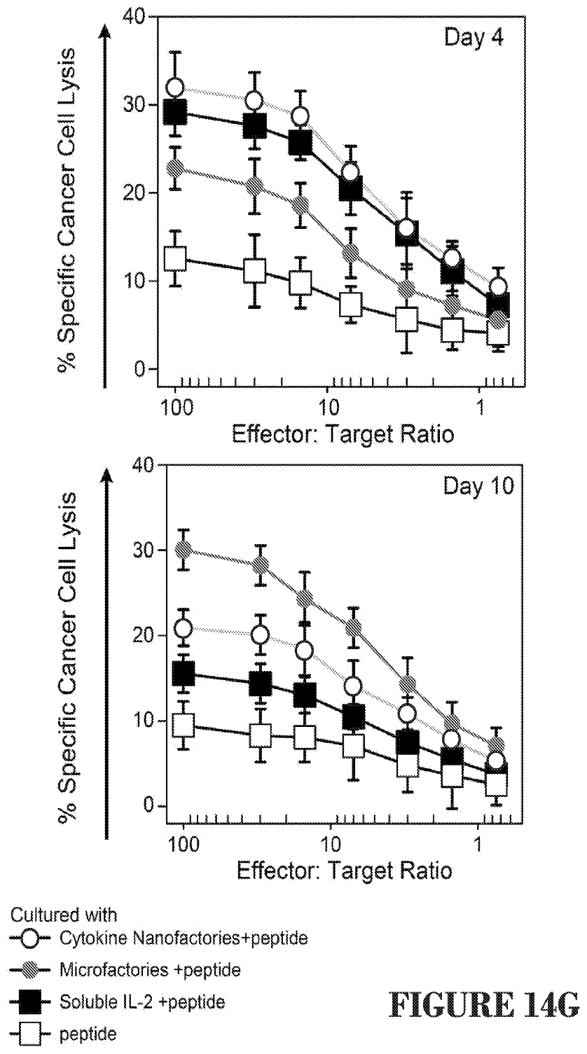

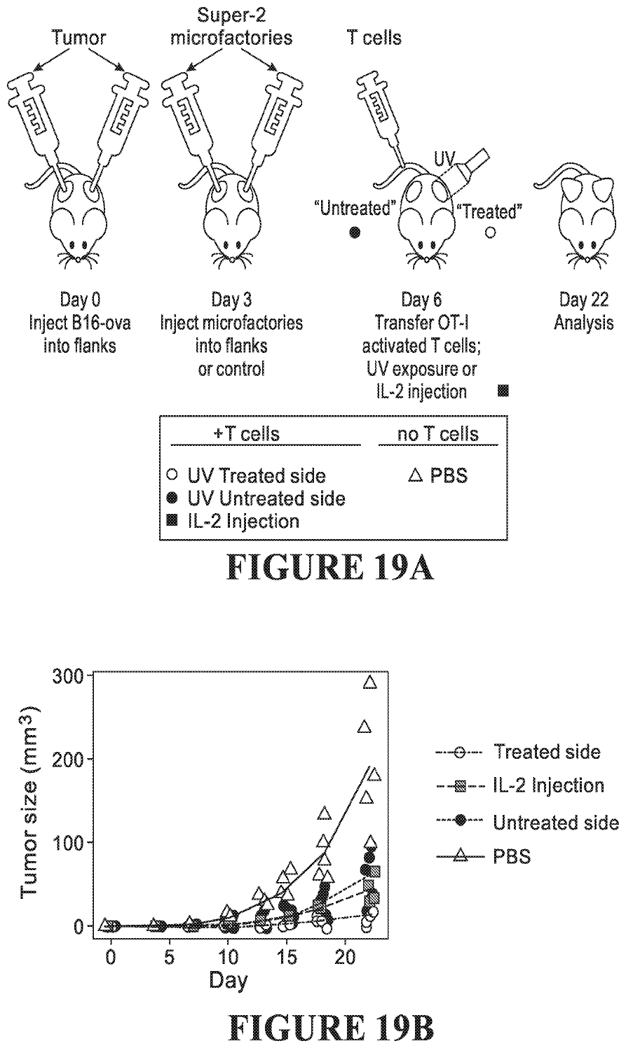

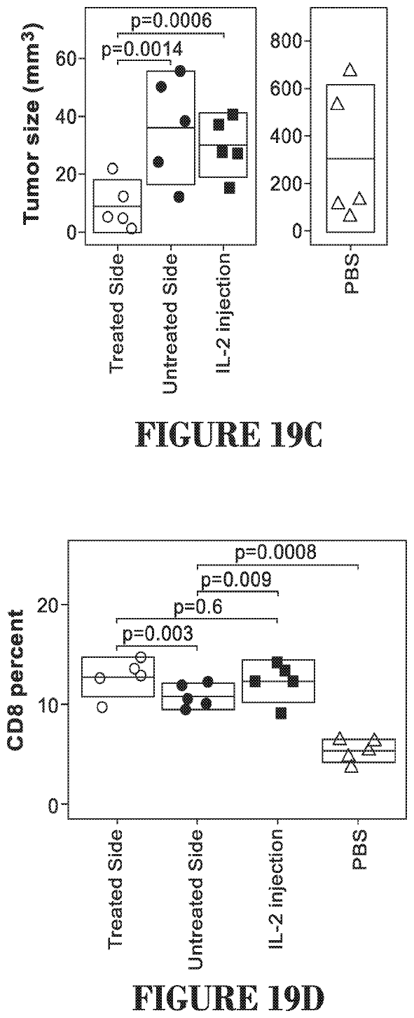

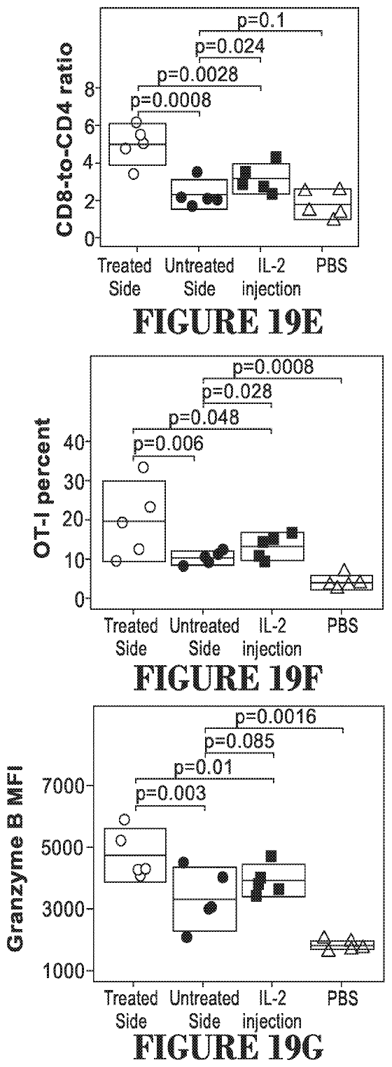

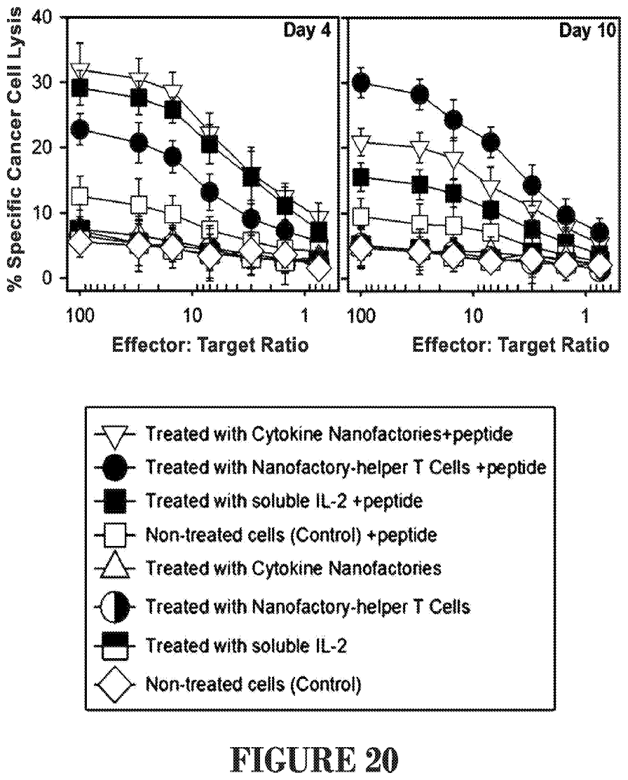

TABLE 3
Details of Particle Formulations Used

| | | | | | Stability | | |
|---|---|---|---|---|---|---|---|
| | | | | | (days) | | |
| Sample | Size* | | Morphology | Method | ** | *** | Cited in FIG. |
| μF-1 | 45 ± 12 | nm | Spherical | DLS | 9-14 | 7-12 | FIGS. 12B-12D |
| μF-2 | 94 ± 28 | nm | Spherical | DLS | 9-14 | 7-12 | FIGS. 12B-12D |
| μF-3 | 166 ± 43 | nm | Spherical | DLS | 9-14 | 7-12 | FIGS. 12B-12D, |
| | | | | | | | 12F |
| μF-4 | 380 ± 90 | nm | Spherical | DLS | 9-14 | 7-12 | FIGS. 12B-12F, |
| | | | | | | | FIGS. 14A-14G |
| Bulk-1 | 191 ± 122 | nm | Spherical | DLS | 6-10 | 6-12 | FIGS. 12B-12D, |
| | | | | | | | 12F |
| Bulk-2 | 371 ± 184 | nm | Spherical | DLS | 4-7 | 4-6 | FIGS. 12B-12D, |
| | | | | | | | 12F |
| Liposome (μF-4) | 5.8 ± 1.0 | μm | Spherical | Microscopy | >30 | >21 | FIGS. 14A-14G |
| encapsulated | (microns) | | | | | | |
| (Alginate) + lipid | | | | | | | |
| membrane | | | | | | | |
| Liposome (μF-4) | 5.1 ± 0.6 | μm | Spherical | Microscopy | >30 | >21 | FIGS. 14A-14G |
| encapsulated | (microns) | | | | | | |
| (Alginate- | | | | | | | |
| Heparin) | | | | | | | |
| Liposome (μF-4) | 6.4 ± 0.9 | μm | Spherical | Microscopy | >30 | >21 | FIGS. 13B-13D, |

| | | |
|---|---|---|
| encapsulated | (microns) | FIGS. 14A-14G, |
| (Alginate- | | FIGS. 19A-19G |
| Heparin) + lipid | | |
| coated | | |

*Average hydrodynamic size
** in PBS supplemented with physiological calcium
*** in T cell media (RPMI/10% FBS)

Dynamic light scattering (DLS) measurements were performed using a ZETASIZER™ (ZETASIZER™ 3000 HS, MALVERN INSTRUMENTS™ LTD., Worcestershire, UK) in backscattering mode at an angle of 173° for water diluted systems.

### Microparticle (Cell Mimicking Particles, “Microparticle Factories”) Formation.

Alginate-heparin conjugate was synthesized using 1-ethyl-3-(3-dimethylaminopropyl)carbodiimide hydrochloride (EDC)-N-hydroxysuccinimide (NHS) (EDC/NHS) carbodiimide crosslinker chemistry and via ethylenediamine (Majedi, F. S. et al. Cytokine Secreting Microparticles Engineer the Fate and the Effector Functions of T-Cells. Advanced Materials 30, 1703178 (2018)). (1-Ethyl-3-(3-dimethylaminopropyl)carbodiimide (EDC, EDAC, or EDCI) is a water-soluble carbodiimide used in combination with N-hydroxysuccinimide (NHS) for the immobilization of large biomolecules.) The amount of conjugated heparin was optimized to provide enhanced affinity toward cationic proteins like IL-2 cytokine. Microfluidic droplet junction chip (glass hydrophilic; channel depth of 100 μm; DOLOMITE MICROFLUIDICS™, Charlestown, Mass.) was utilized to make monodispersed alginate and alginate-heparin microparticles. Alginate-Heparin solution (10 mg/mL) was mixed with nanoliposomes (2.5 mg/mL) and used as the inner aqueous phase. Mineral oil containing nonionic surfactant (10 wt % SPAN® 80 [SIGMA-ALDRICH™ #S6760) was used as the continuous phase. Flow rates of 6.5 and 14 microliters (4)/min were applied using two syringe pumps (HARVARD APPARATUS PHD™ 2000) for the polymer and oil flows, respectively. Images were taken at various time points using a NIKON™ inverted microscope to check and tune the flow properties. The formed particles were collected in a bath of calcium ions (100 mM CaCl2 [CaCl₂]) and left at room temperature for 30 mM in the dark for ionic crosslinking. Inserting of magnetic nanoparticles (SPION™; 50 nm, carboxylated, CHEMICELL™ GmbH) was also utilized to facilitate separation and washing of particles after formation. The microgels were extensively washed with 20 mM NaCl solution and centrifuged (15,000 rpm for 15 min) twice before further use.

### Microparticle Coating

For the preparation of lipid-coated alginate-heparin microparticles, POPC phospholipid was dissolved in chloroform. Rotary evaporation of the phospholipid was used to form a homogeneous lipid film. The film was further dried under nitrogen and followed by high vacuum overnight. The pre-warmed dried lipid was then wetted using microparticles containing PBS at 60° C. and agitated at 37° C. for 15 min. Samples were cooled to room temperature and then centrifuged to remove extra lipids and re-dispersed in PBS for further use. Depending on the composition, the dried lipid is warmed to about 40° C. to about 90° C.

### Protein Production and Detection

The solutions of free nanoliposomes or encapsulated inside alginate(-heparin) microparticles were kept at 4° C. before the UV triggering. To activate the protein production machinery, UV light (360-480 nm) was used to illuminate the particles for 10 s at 80 mW/cm² (pH 7.4 and 37° C.). Confocal imaging was used to visualize synthesis of green fluorescence protein (GFP) inside the nanoliposomes. A 100×PLAN APO™ numerical aperture (NA) with 1.4 objective (NIKON™) used for the fluorescent imaging.

### Protein Production in 3D Scaffold

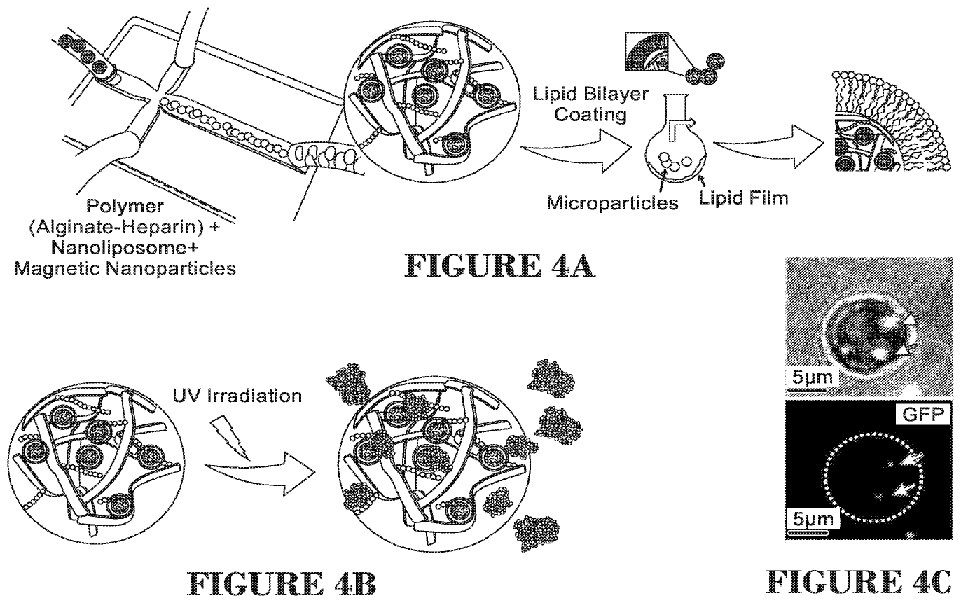

To investigate whether these nanofactories can produce proteins in 3D, Alginate-based scaffold was made using freeze-drying process. Alginate forms hydrogels and addition of Arg-Gly-Asp (RGD) motifs improves cell adhesion after printing. The alginate was dissolved in 2-morpholin-4-ylethanesulfonic acid (MES) (MES 150 mM, NaCl 250 mM, pH 6.5) and covalently conjugated to RGD-containing peptide (GGGGRGDY [SEQ ID NO: 8]; GENSCRIPT™ USA Inc., Piscataway, N.J.) using carbodiimide chemistry (EDC/NHS). The solution of alginate-RGD (20 mg/mL) in PBS was mixed with 25 mM calcium sulfate and was poured in a pre-made PDMS mold, frozen at −80° C., lyophilized for 3 d, and stored at 4° C. EDC/NHS chemistry used to post-conjugate scaffolds surface with nanoliposomes. The scaffold irradiated with the UV source as mentioned in the previous sections and confocal imaging of the produced GFP monitored 75 after UV exposure (see FIGS. 4A-4C).

Scanning electron microscopy (SEM) was used to observe the cross-sectional microstructure of the alginate 3D scaffolds. Lyophilized scaffolds were freeze-fractured (using liquid nitrogen) to expose a cross-section. The scaffold specimens were imaged without further coating using a ZEISS SUPRA™ 40 VP scanning electron microscope (CARL ZEISS MICROSCOPY™ GmbH, Jena, Germany).

### In Vitro Functional Assays

### T Cell Isolation and Activation

Five- to eight-week-old wild type mice were purchased from the University of California, Los Angeles (UCLA) and maintained in pathogen-free facilities at UCLA. All experiments on mice and cells collected from mice were performed in strict accordance with UCLA's institutional policy on humane and ethical treatment of animals Cell culture media was Roswell Park Memorial Institute (RPMI) supplemented with 10% heat inactivated fetal bovine serum (FBS), 100 micrograms (μg)/mL streptomycin, 100 U/mL penicillin, 50 micromolar (uM) beta-mercaptoethanol (2-mercaptoethanol; (3-ME, 2-ME). Total T cells, CD4+ T cells or CD8+ T cells were purified from spleens using negative enrichment kits (APPYMETRIX EBIOSCIENCE™).

For in vitro activation of purified T cells (CD8+ or CD4+), 96-well plates were prepared with anti-CD3 (2C11; BIO X CELL™) coating plates at a concentration of 5 micrograms (μg)/mL overnight, followed by washing with PBS. T cells at a concentration of 1×10⁶/mL (1×106/mL) with 2 micrograms (μg)/mL soluble anti-CD28 (37.51; BIO X CELL™) in the presence of nanoliposomes (free or encapsulated in microparticles) or 100 IU/mL of soluble human IL-2. Microparticles were added at a 1:1 ratio of particles to cells, or else the equivalent number of nanoliposomes (around 400 nanoliposomes/microparticle) was added to cell media as free nanoparticle control. For flow cytometry analysis, antibodies to mouse CD8 (53-6.7), CD25 (PC61.5), CD44 (IM7), CD62L, and CD16/CD32 (FC block) were purchased from EBIOSCIENCE™, BIOLEGEND™, or BD BIOSCIENCES™ Propidium iodide and acridine orange were purchased from CALBIOCHEM™. Cells were analyzed on a CYTEK DXP™ Flow Cytometer using FLOWJO™ software (TREESTAR™).

To study the in vitro release profile of produced cytokine, nanoparticle-loaded microparticles were dispersed in PBS (pH 7.4) and exposed to UV for 10 s. 500 microliters (μL) of microsphere dispersion were placed in EPPENDORF™ tubes, gently shaken, and incubated at 37° C. At predetermined time points, samples were collected using centrifugation, and the supernatant were replaced with an equivalent volume of fresh PBS solution. The concentration of released cytokine from microparticles was determined using plate reader. To detect the production of protein, the FLUOROTECT™ GREENLYS™ tRNA (PROMEGA™) was included in the nanoliposomes, replacing a portion of the available lysines with a fluorescently-tagged amino acid during synthesis. This tRNA is labeled with BODIPY™-FL (boron-dipyrromethene fluorophore; 1,3,5,7-tetramethyl-8-phenyl-4,4-difluoroboradiazaindacene; difluoro {2-[(3,5-dimethyl-2H-pyrrol-2-ylidene-N)phenylmethyl]-3,5-dimethyl-1H-pyrrolato-N}boron) dye, which has an excitation maximum of 502 nm and an emission maximum of 510 nm. To eliminate the signal arising from unincorporated green lysine, the nanoparticles were pelleted and only the supernatant was collected. The supernatant is the fraction containing diffused cytokine, but it can also contain fluoro-lysine (fluoro-Lys). The supernatant samples were then filtered using AMICON ULTRA™ centrifugal filter units (ULTRA-4™, molecular weight cutoff [MWCO] 5 kDa). Free fluoro-Lys is significantly smaller than 5 kDa. The concentration of fluorescent cytokine was determined using a plate reader.

The synthesized IL-2 (Super2) used here is different from wildtype IL-2, and so the use of a commercial IL-2 ELISA to rule out detection of tRNA or its decomposition products was not possible. In FIG. 14A, secreted Super2 that diffuses out of the nanoliposomes was detected. To eliminate the possibility of the signal arising from unincorporated green lysine, the nanoparticles were pelleted and only the supernatant—the fraction that should contain diffused Super2 but also could contain fluoro-Lys—was taken. The samples were filtered using an AMICON ULTRA™ centrifugal filter units (ULTRA-4™, MWCO 5 kDa). Free fluoro-lysine (fluoro-Lys) is much smaller than 5 kDa. The fluorescence of the solution was then measured. This process eliminated the potential contamination of the Super2 measurement.

### Chromium Functional Assay

Transgenic OT-I mice (5-10 weeks old) were used for in vitro cytotoxicity assay and were kept under specific pathogen-free conditions, according to institutional guidelines. B16F10-ova and B16F10 were cultured in Dulbecco's Modified Eagle's Medium (DMEM) that contained 5% fetal calf serum (FCS) and glutamine, with penicillin/streptomycin added to it. OT-I CD8+ T cells were purified by negative selection as mentioned before, and co-cultured with various formulations of designed particles microparticles at a one-to-one ratio of microparticles. At the end of the culture period (day 4 or day 10), T cells were recovered. Finally, a total of 5×10⁶ (5×106) transgenic T cells/well in a volume of 1 mL were counted and serially diluted up to seven times (T Cell: tumor cell of 100:1, 30:1, 15:1, 7:1, 3:1, 1.5:1, 0.75:1) in Minimum Essential Medium (MEM) supplemented with 2% FCS. Specific cytotoxicities of the dilutions were then determined in a standard 51Cr (⁵¹Cr) release assay, as described (Hany, M. et al. Anti-viral protection and prevention of lymphocytic choriomeningitis or of the local footpad swelling reaction in mice by immunization with vaccinia-recombinant virus expressing LCMV-WE nucleoprotein or glycoprotein. European Journal of Immunology 19, 417-424 (1989)), and 10⁴ cells were added to the effector cells in a final volume of 200 μl. After a 12 h incubation at 37° C., 60 microliters (μL) of the supernatant were harvested and counted with a gamma counter (PERKINELMER™, Richmond, Calif.).

To quantify the GFP produced over time, we found that 2×106 microfactories can produce ca. 8.7±2.1 ng GFP after one hour after UV exposure in vitro.

### In Vivo Functional Assays

### In Vivo Protein Production

5×10⁶ (5×106) protein producing microparticles (150 microliters [μL]) subcutaneously injected into right flank of C57BL/6J wild-type (WT) mice (6-8 weeks old) mice. 24 h after the injection the injection sites divided in half. One half covered with ultraviolet (UV) absorbing film and the other half subjected to UV irradiation at 300 mW/cm² power using a 365 nm UV source (OMNICURE™ 52000) for 300 s. Protein (GFP) production was visualized using whole animal fluorescence scanning 3 h post irradiation.

### In Vivo Tumor Suppression Assay

2×10⁵ (2×105) B16F10-OVA cells subcutaneously injected into both right and left flanks of C57BL/6J wild type (WT) mice (6-8 weeks old) mice (Weigelin, B. et al. Focusing and sustaining the antitumor CTL effector killer response by agonist anti-CD137 mAb. Proceedings of the National Academy of Sciences 112, 7551-7556 (2015)). These melanoma-derived cells are transfected to express chicken ovalbumin peptide (OVA) (Bellone, M. et al. Relevance of the tumor antigen in the validation of three vaccination strategies for melanoma. The Journal of Immunology 165, 2651-2656 (2000)). Three days after tumor cell injection, 5×10⁶ (5×106) Super2 microfactories (alginate-heparin microparticles encapsulating protein producing nanoparticles) were adoptively transferred subcutaneously into the same approximate region of the tumors in both flanks. 500×10³ (500×103) OT-I T cells were activated ex vivo with anti-CD3/anti-CD28 3 days and then transferred intravenously on day 6 by retro-orbital injections (100 microliters [μL] per animal) That same day, right flanks were exposed to UV for 300 s using OMNICURE™ 52000 (365 nm; 300 mW/cm²). On the same day, control animals (sides) were injected with soluble IL-2 (5,000 units/kg) at sites immediately proximate to the tumor.

Tumor size was assessed over time using a digital caliper until day 22 at which animals sacrificed and the tumor, spleen and draining lymph nodes (dLNs) were extracted. Tumor mass was measured using a digital balance before digesting the tumor tissue for flow cytometry. Tissues were digested by incubating in collagenase and DNase I (50 micrograms (μg)/mL) at 37° C. for 15 min (Weigelin, B. et al. ibid). These enzymes were inactivated with ethylenediaminetetraacetic acid (EDTA) (20 microliters [μL]/mL of solution). Tissues then were mechanically disaggregated and passed by a 0.7 micron (μm) cell strainer to obtain a single-cell suspension. Cells were then stained with the fluorochrome-conjugated antibodies on ice. For intracellular staining (e.g., Granzyme B), cells were permeabilized with FOXP3 FIX/PERM™ buffer according to manufacturer instructions (BIOLEGEND™) before staining

### Nomenclature

As used herein in the Examples below, the terms “nanofactory” and “nanofactories” describes protein producing nanoliposomes. Loading (encapsulation) of these nanofactories inside alginate or alginate-heparin microparticles results in the formation of microfactories. A “Super2 nanoliposomal factory” are those nanoliposome/microparticles expressing IL-2 superkine.

Microparticles were added in a 1:1 ratio of particles to cells and the equivalent number of nanoliposomes (around 400 per cell) was added to cell media as free nanoparticle control.

### Statistics

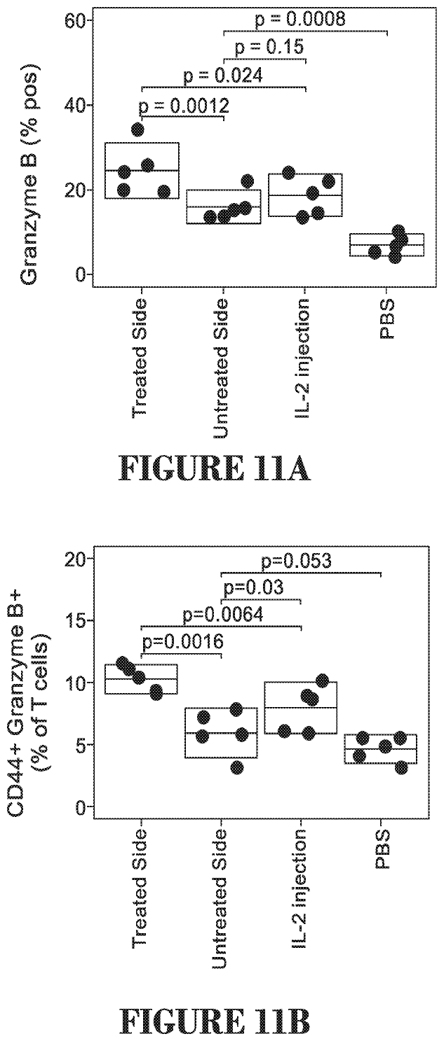

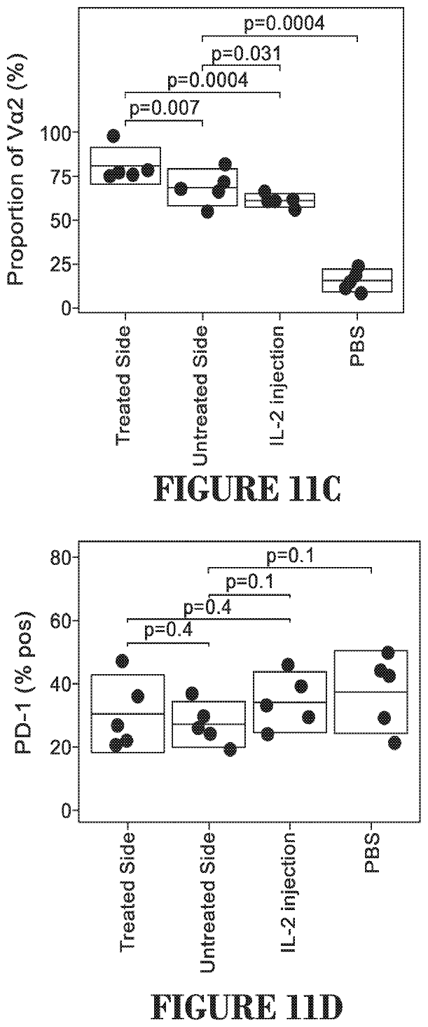

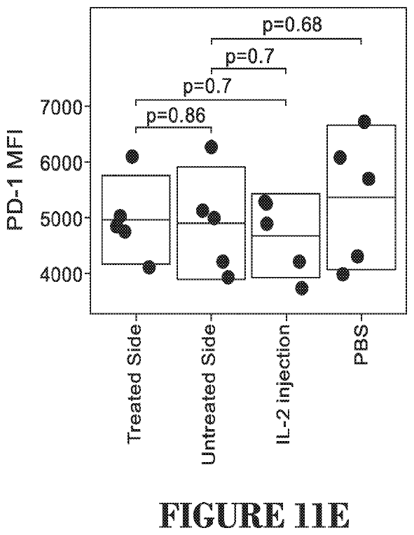

Permutation testing was used for all statistical comparisons of flow cytometry data, survival curves, fluorescence colocalization, and stiffness. This method reduces the potential influence of outliers and relaxed the requirement of knowing the distribution of observations by comparing the value of the test statistic to a reference distribution generated from the data themselves, rather than to a standard distribution (L. M. Chihara and T. C. Hesterberg, Mathematical statistics with resampling and R, John Wiley & Sons, Hoboken, N.J., 2018). To ensure that permutation testing was a suitable way to compare means, it was first shown that the variances of the two groups were similar by the non-parametric Ansari-Bradley test. The permutationTest2 function of the “resample” package of R was used to calculate two-sided p-values and determine the 95% confidence intervals, performing typically 50,000 permutations. All average values are bootstrapped means, calculated using the “bootstrap” function of the resample package in R. All boxes in figures show the bootstrapped mean and the calculated 95% confidence interval. Confidence intervals are calculated using the “CI.t” function of the resample package in R.

Where multiple groups were compared (e.g., FIGS. 11A-11E, FIGS. 19A-19G), the comparison was found significant first by ANOVA where treatment was compared to the outcome measure (e.g., tumor mass). Then, post-hoc pair-wise comparisons are shown with p-values calculated by permutation testing as above. Adjustment of multiple comparison testing p-values was done by the Benjamini-Hochberg method using the p.adjust function in R.

### Example 15: Production of Nanoliposomes

Objective: To produce nanoparticles that can encapsulate a cell-free transcription and translation machinery. This approach is predicated on the nanoencapsulation of cell-free transcription and translation machinery, coupled with a mechanism to control protein production (FIG. 12A).

Methods: To encapsulate synthetic machinery, the first step was to encapsulate cell-free protein biosynthesis systems (also known as in vitro transcription-translation kits) into liposomes to contain the reagents. A microfluidics approach was used to prepare nanoliposomes (Schroeder et al., Nano Lett., 2012, 12, 2685-2689; Hasani-Sadrabadi et al., Adv. Mater., 2016, 28, 4134-4141). The nanoliposomes encapsulated the machinery of in vitro transcription and translation systems, and any desired plasmids.

Results: The approach used in developing the nanoparticles described herein and there use in cancer or tumor therapy is predicated on a nanoencapsulation of cell-free transcription and translation machinery, superimposed with mechanisms to control protein production. A broad size range of nanoliposomes (50-400 nm) and narrow polydispersity was achieved by adjusting relative microfluidic flow rates (FIGS. 12B-12C).

To test whether scaling the size of liposomes could affect their ability to hold cargo, three sizes of plasmids (3300-6500 bp) were encapsulated within six sizes of nanoliposomes (40-400 nm), and encapsulation measured. Plasmid sizes tested were 3320 bp, 5280 bp, and 6565 bp, while liposome sizes analyzed were μF-1, μF-2, μF-3, μF-4, Bulk-1, and Bulk-2 (See TABLE 2 and TABLE 3). (Bulk-1 and Bulk-2 were not made by the microfluidic droplet generator.) The efficiency of loading depended on the sizes of both the cargo and the nanoliposomes (FIG. 12D). In contrast to microfluidic approaches, formation of nanoparticles by conventional bulk mixing and extrusion techniques with porous membranes (100-800 nm) produces polydisperse particles of 100-800 nm diameter (FIG. 12C), and thus could incur variable inefficiencies in loading plasmids.

Conclusion: Based on these data, nanoliposomes of 350 nm diameter (“uF-4”) were able to encapsulate substantial amounts of all three plasmids and were selected for further investigation.

### Example 16: Temporal Control of Protein Production in Nanofactories

Objective: To provide temporal control over protein production in the nanofactories. Eliminating the basal and continuous expression of cytokines is critical to reducing their systemic toxicity.

Methods: As protein-synthetic machinery is ATP-dependent, therefore it was tested whether a UV-caged ATP could control protein synthesis (Monroe et a., J. Biol. Chem., 1999, 274, 20895-20900). Exposure to UV uncages the ATP and should initiate transcription and translation. Transcription/translation machinery provided by cellular extracts, an IL2-GFP plasmid (ADDGENE™ #67053), and caged ATP were encapsulated into nanofactories. The nanofactories were imaged using fluorescence microscopy.

Results Imaging of nanofactories revealed no detectable basal expression of GFP, while UV exposure at 360-480 nm wavelength for 10 s (at 80 mW/cm2; pH 7.4; 37° C.) uncaged the ATP and resulted in robust GFP expression in 30 min (FIG. 12E). FIG. 12E shows a series of micrographs wherein it can be seen that the expression of IL-2-GFP increases over time.

To test the dependence on size, NPs of various sizes were formed and particles with diameters of >100 nm were all capable of producing detectable GFP (Data not shown).

Summary: As GFP only fluoresces if IL-2 and GFP are folded properly, these observations show the fabricated nanoparticles contained all components necessary for transcription, translation, and folding of cytokines on demand. Nanofactories greater than 100 nm in size were capable of producing GFP, and larger nanofactories were more productive (FIG. 12F). Improved encapsulation efficiency of protein production machineries into microfluidic assisted synthesized liposomes resulted in protein synthesis with higher efficiency.

### Example 17: Synthesis of Alginate-Heparin Microfactories

Objective: To endow the liposomal nanofactories (nanoparticles, NP) with additional capabilities, they were microfluidicially encapsulated them into alginate-heparin microfactories (microparticles).

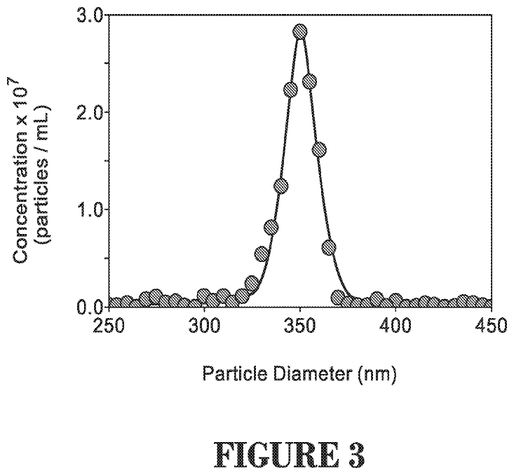

Methods: Liposomal nanofactories were prepared as in Example 16 (see, e.g., Majedi, F. S. et al. Cytokine Secreting Microparticles Engineer the Fate and the Effector Functions of T-Cells. Advanced Materials 30, 1703178 (2018); Hasani-Sadrabadi, M. M. et al. Mechanobiological Mimicry of Helper T Lymphocytes to Evaluate Cell-Biomaterials Crosstalk. Advanced Materials 30, 1706780 (2018)) In order to localize synthesis and controlled delivery of cytokine at tissue level, artificial cells were fabricated that not only mimic the size, shape, and mechanics of lymphocytes like T cells (Hasani-Sadrabadi et al. Adv. Mater., 2018, 30, 1706780), but also contain the synthetic machinery for transcription and translation of functional cytokine, and elements that allow for precisely controlled activation and release of the cytokine. To load the liposomal nanofactories into delivery vehicles with additional capabilities, the nanoliposomes were microfluidically into alginate-based microparticles (FIG. 13A), with an average of 443 nanoliposomes per microparticle (FIG. 3). To facilitate magnetic purification of the “microfactories,” superparamagnetic iron oxide nanoparticles (SPIONs) were embedded during microfluidic synthesis (Schroeder et al., Nano Lett., 2012, 12, 2685-2689).

Nanofactories were prepared with and without alginate-heparin. The heparin-modified alginate was used as a bulk material to make microparticles and encapsulate nanofactories. The effects of heparin inclusion were also tested. So, there is a group of microparticles based on alginate or alginate-heparin, wherein these microparticles encapsulate nanoparticles (nanofactories) inside them.

To facilitate magnetic purification of the “microfactories,” superparamagnetic iron oxide nanoparticles (SPIONs) were embedded during microfluidic synthesis (Schroeder et al., Nano Lett., 2012, 12, 2685-2689).

Results: The methods used delivered microparticles comprising on average 400 nanoparticles per microparticle. Microparticle factories showed no basal expression of IL2-GFP. Upon UV-illumination, protein synthesis from the microparticle factories plateaued at ˜60 min (FIGS. 13B-13C; see also FIG. 12F).

### Example 18: Delivery and Activation of Microfactories In Vivo

Objective: To demonstrate the capability of the microfactories to be delivered and activated in vivo.

Methods: Mouse skin was “tattooed” with interleukin-2/green fluorescent protein (IL-2-GFP) microfactories (produced as in Examples 16 and 17), half the area was taped (covered), and the tattooed skin was partly exposed to UV light (FIG. 13D). These wavelengths of light allow for ˜1 mm of penetration in the epidermis (Meinhardt et al., J. Biomed. Opt., 2008, 13, 044030).

Results: Within 60 min of exposure to UV light, GFP production was evident from the exposed area but not from the covered portion (FIG. 13D).

Summary: The results confirmed that microfactories can survive in vivo and can produce functional protein after UV activation.

### Example 19: Biological Effects of Synthesized Cytokines on T Cells In Vitro

Objective: To test the biological effects of synthesized cytokines, from nanofactories or microfactories, on T cells.

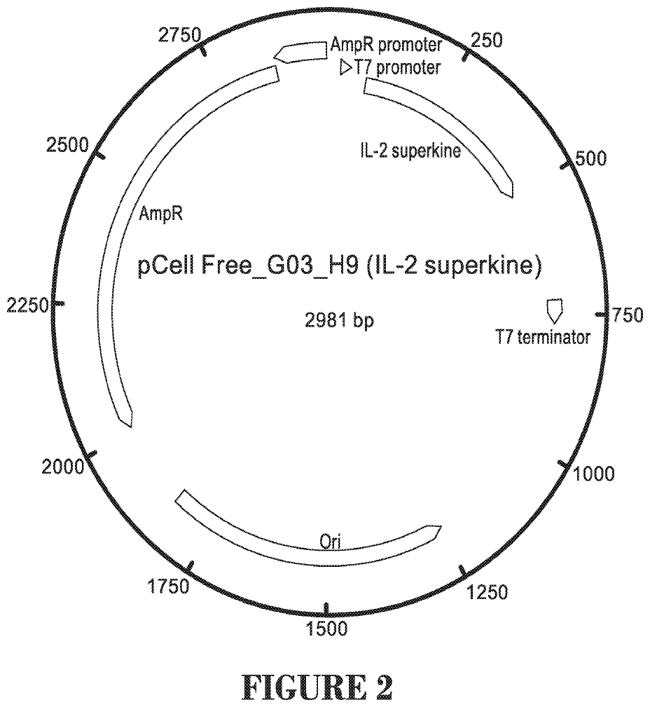

Methods: IL-2 has been FDA-approved for treatment of several cancers since the 1980s (Lotze et al., Cancer, 1986, 58, 2764-2772), but clinical use has been limited due to severe side effects, some of which include vascular leak syndrome, hypotension, and cardiac toxicity (Jiang et al., Oncoimmunology, 2016, 5, 1-10; Zhang et al., Nat. Commun, 2018, 9, 6). Here, a synthetic IL-2 (Super2) (FIG. 2) was used, which has the advantage of activating T cells expressing the receptor most commonly found on naïve T cells—comprising IL2Rβ (CD122) and IL2Rγ (CD132)—but not requiring the high affinity IL2Rα chain (CD25) that is found on activated and regulatory T cells (Levin et al., Nature, 2012, 484, 529-533).

The IL-2 ‘superkine’ was synthesized by using the vector constructed as shown in FIG. 2. Naïve T cells were co-cultured with Super2-producing nanofactories, either as free nanoparticles, as nanofactories encapsulated in alginate microfactories. For some particles, alginate was conjugated with heparin 0 before encapsulation to test whether heparin could prolong release of the cytokine. For others, the microfactories were coated in a lipid membrane comprising 1-palmitoyl-oleoyl-sn-glycero-phosphocholine (POPC) for a similar test.

Cytotoxic MHC class I-restricted, ovalbumin-specific, CD8+ T cells (OT-I T cells) were cultured with microfactories for 3 days, then fluorescently-labeled B16-ova melanoma cells that bore the cognate antigen for T cells were added Killing of melanoma cells was measured by flow cytometry.

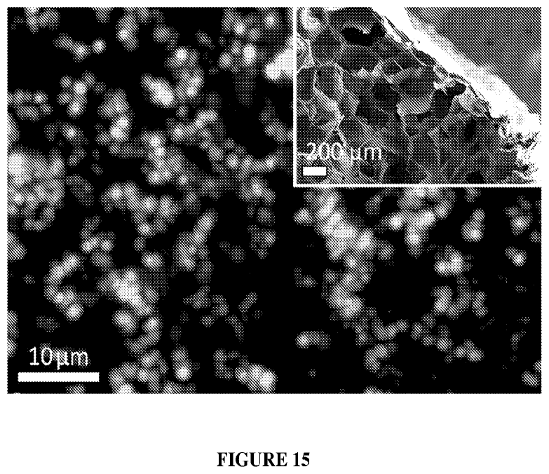

Results: FIG. 15 shows the structure of an alginate-RGD scaffold encapsulating IL2-GFP-producing NPs. These microfactories included both alginate-heparin and POPC.

We showed that the duration of Super2 release increased over 10-fold by heparin incorporation and lipid encapsulation, with 50% release extended from 5.4 h to 62 h (FIG. 14A). These encapsulations can provide sustained delivery of produced Super2 (FIG. 14A) from both nanofactories and microfactories.

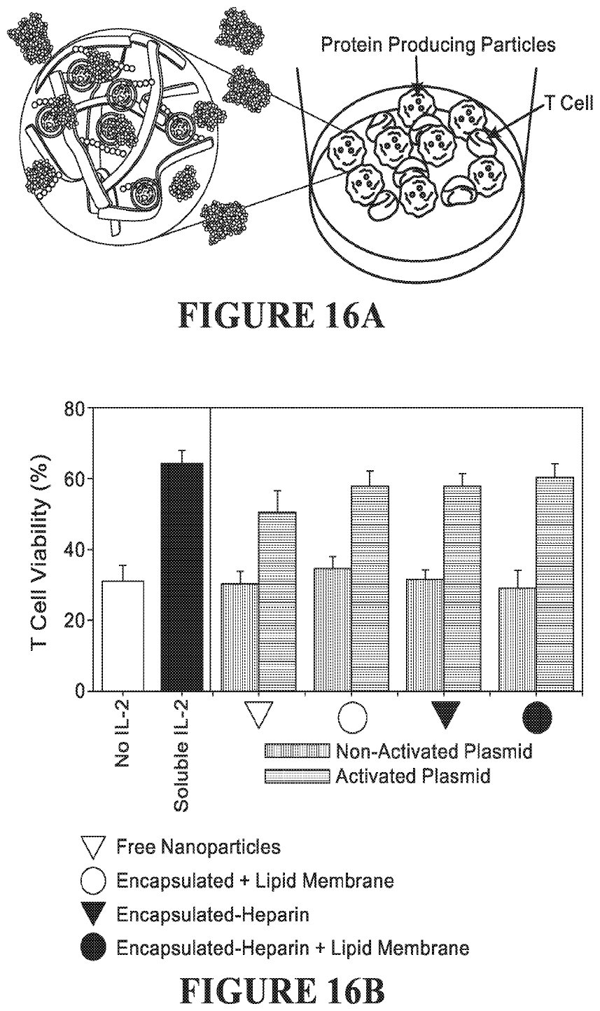

To assess the impact on T cells, in vitro proliferation was measured on days 4 and 10, and it was found that free nanofactories (fastest cytokine release) elicited the highest expansion early (day 4), but heparin-lipid encapsulation of nanofactories (slowest cytokine release) resulted in the greatest sustained expansion (p<0.005 compared to each other condition) (FIG. 14B, FIGS. 16A-16B).

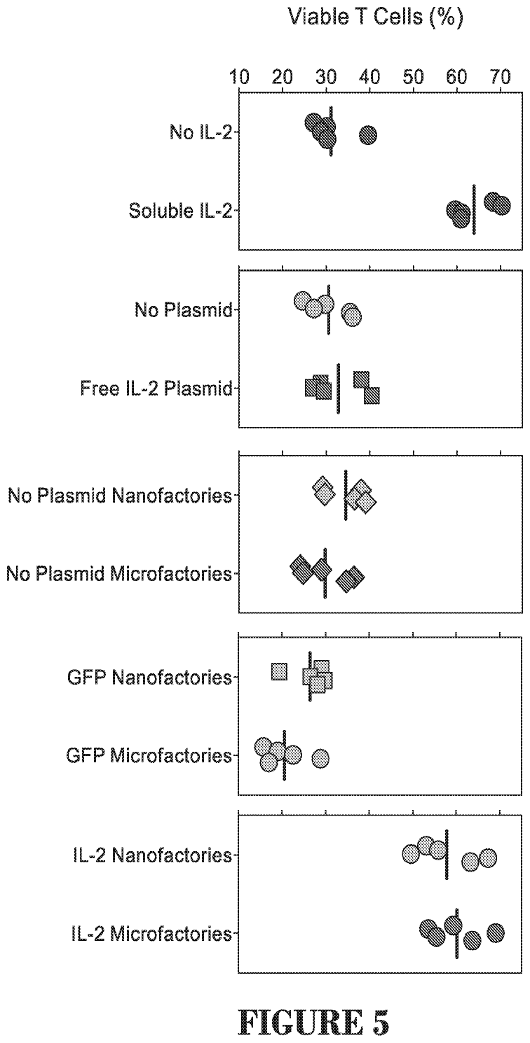

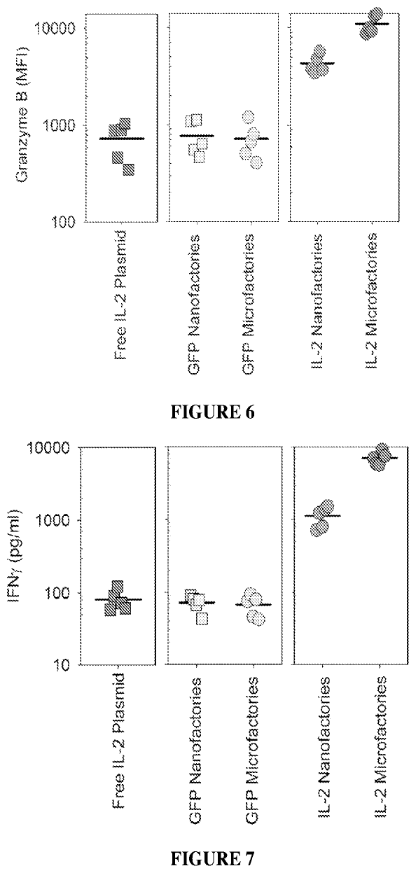

To study the mechanism underlying enhanced proliferation, it was found that enhanced viability (FIG. 14C, FIG. 5), expression of Granzyme B (FIG. 14D, FIG. 6), and IFN-γ secretion (FIG. 14E, FIG. 7) correlated with enhanced proliferation.

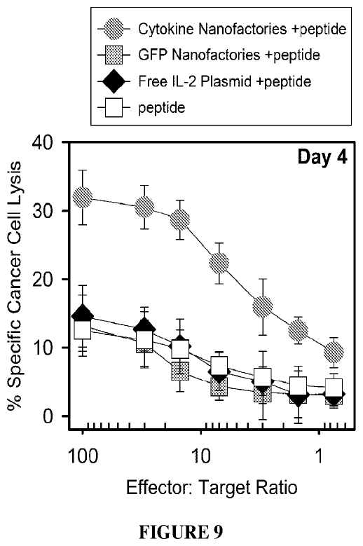

To eliminate the possibility that the presence of the CG dinucleotide repeats in DNA in the plasmid could be an adjuvant through TLR9, the effects of free Super2 plasmid and GFP plasmid were compared in the viability of cultured T cells (FIG. 5). Free Super2 plasmid and GFP plasmid did not improve the viability of T cells. The CG dinucleotides, those contiguous pairs of DNA that could potentially be a ligand for TLR9, in the Super2 plasmid and the GFP plasmid, were assessed. The GFP plasmid had a total of 483 CG dinucleotides and the Super2 plasmid had a total of 348 CG dinucleotides—less than the GFP plasmid—and yet survival is higher in T cells exposed to the Super2 microfactories than the GFP microfactories (FIG. 5). These results support that the DNA of the plasmid itself is not acting as a survival signal, through TLR9 or other mechanisms Similar comparisons for the GFP control were found for viability (FIG. 5, FIG. 14C), Granzyme B expression (FIG. 6, FIG. 14D), IFN-gamma (IFN-γ) expression (FIG. 7, FIG. 14E), and cytotoxic killing (FIG. 9, FIG. 14G).

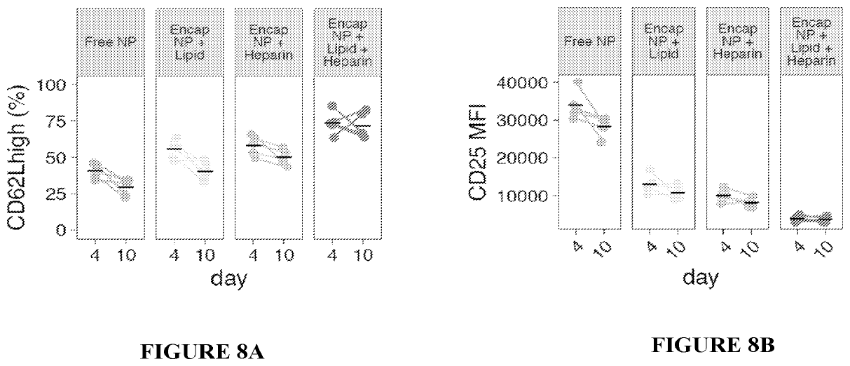

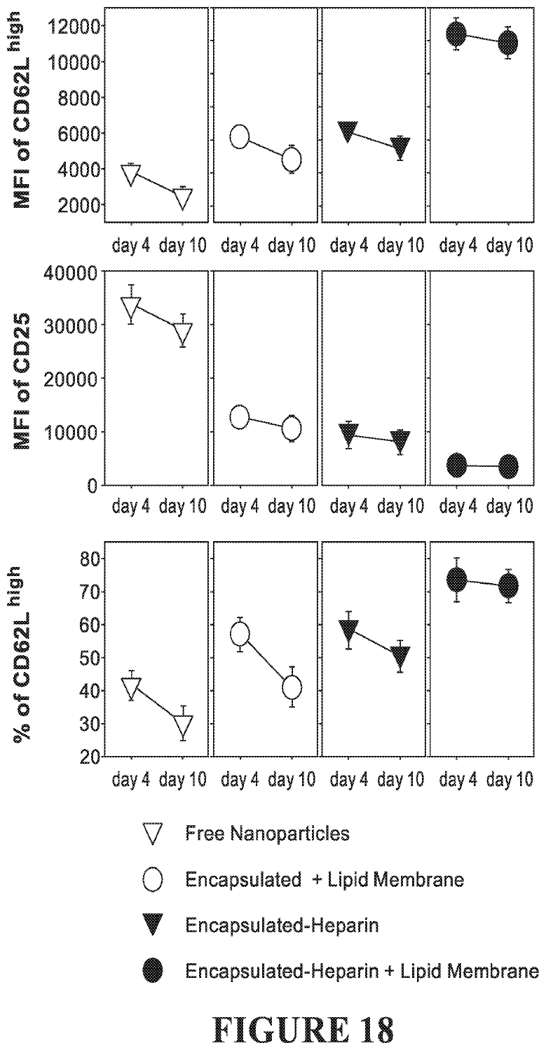

Sustained release of synthetic IL-2 resulted in a potent effector pool but modest upregulation of CD25 and minimal downregulation of CD62L (FIG. 14F, FIG. 8A-8B, FIG. 18). In contrast, exposure to rapidly synthesized Super2 by free nanofactories promoted downregulation of CD62L and upregulation of CD25. Thus, sustained protein synthesis and exposure to Super2 generated a differentiation state of effector T cells with strong killing potential.

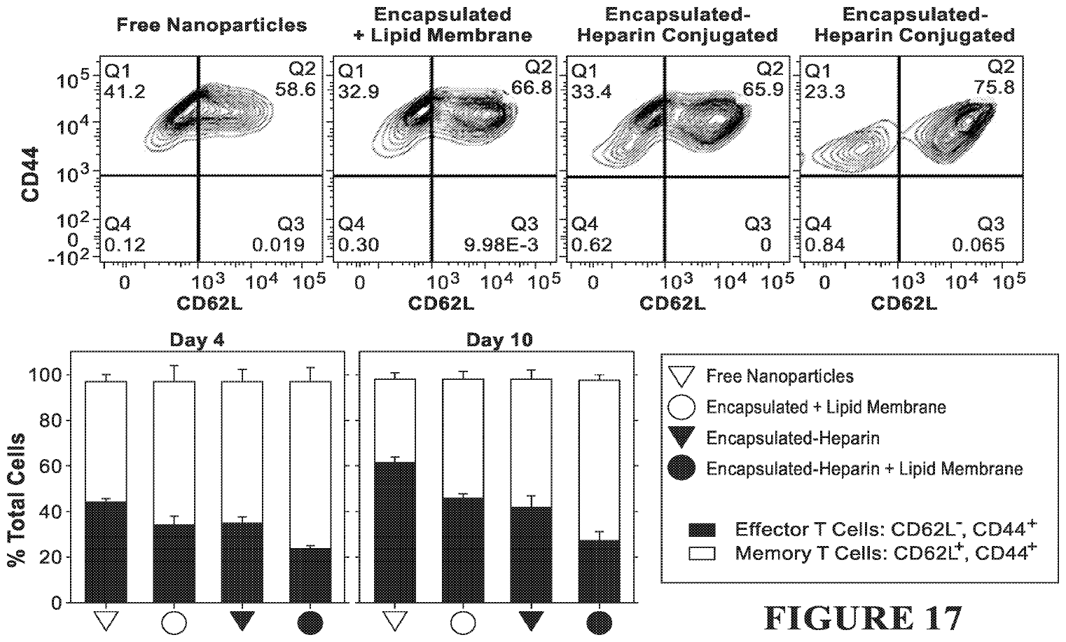

To test whether accentuation of T cell activation by the microfactories could also augment antigen-specific killing of cancer cells, naïve OT-I T cells were cultured with Super2 microfactories for 3 days, then fluorescently-labeled B16-ova melanoma cells were added as targets. T cells co-cultured with microfactories showed enhanced killing as compared to soluble IL-2 (FIGS. 14F-14G, FIG. 9). It was found that T cells co-cultured with microfactories sustained their killing activity for longer period of time (FIG. 14G; FIG. 17).

Summary: Taken together, these results demonstrate that T-cell activation, proliferation, and killing were enhanced by Super2 synthesis from microfactories. The survival of T cells in vivo and in vitro is highly dependent on the presence of IL-2 and not only on the chemistry and composition of particles.

### Example 20: Effect of Microfactories on Cancer Killing In Vivo

Objective: To test the impact of microfactories on cancer killing in vivo. Nanofactories are used to describe protein producing nanoliposomes. Loading (encapsulation) of these nanofactories inside alginate or alginate-heparin microparticles caused formation of microfactories. The goal was to examine the effectiveness of these microfactories in vivo.

Methods: Mice were subcutaneously injected with B16-ova melanoma cells on both flanks (FIG. 19A). After the tumors became palpable, Super2 microfactories were injected subcutaneously into the same regions as the tumors, and activated OT-1 T cells were transferred intravenously into the mice. Only one flank was illuminated by UV light to activate protein production (FIG. 5K). As a control, intratumoral injection of soluble IL-2 was given to some mice. Growth of the tumors were measured until day 22, at which point untreated mice carried a tumor burden that required euthanasia. All mice were then euthanized, and the tumor and regional lymph nodes were subjected to flow cytometry.

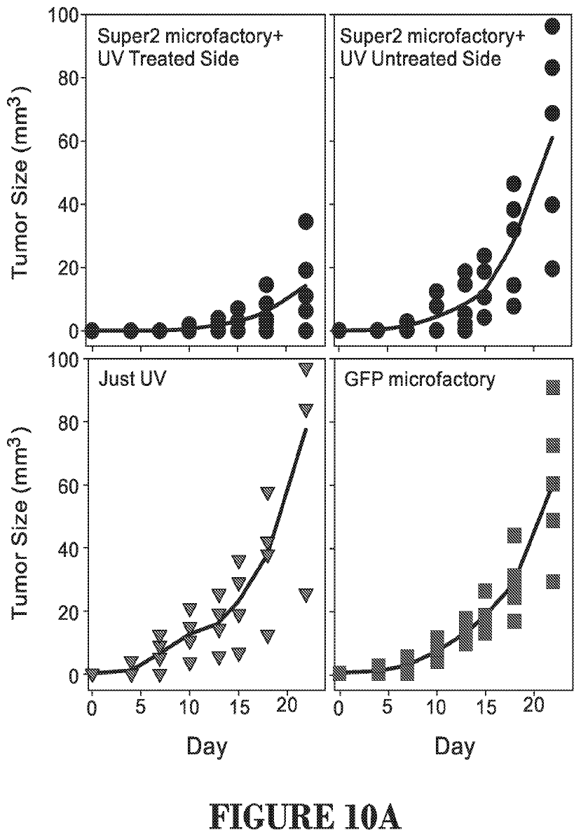

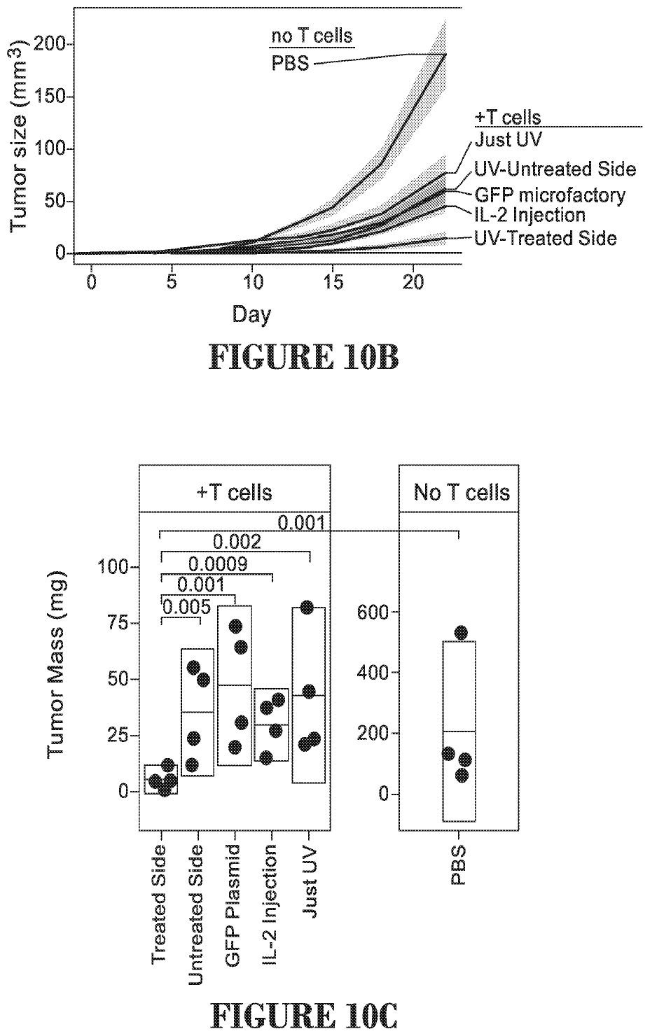

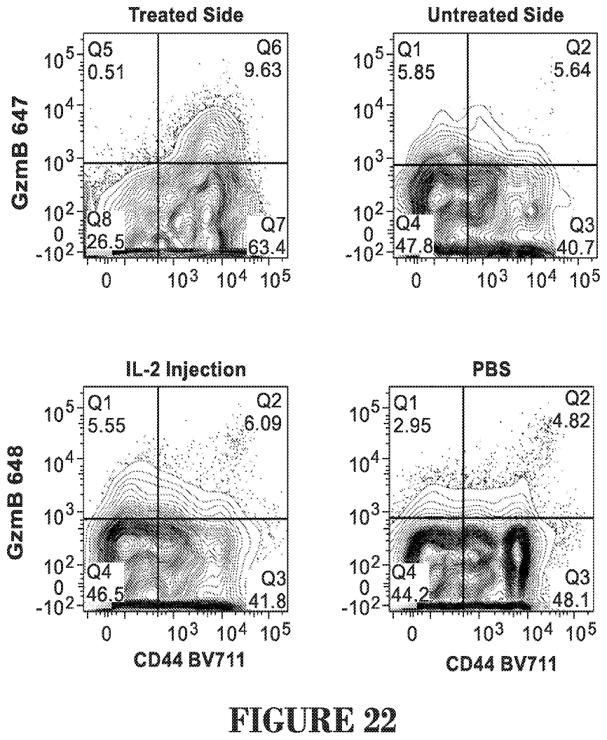

Results: The UV exposed side showed virtually no growth of the tumor (FIGS. 19B and 19C, FIGS. 10A-10C), whereas the unilluminated side showed substantial growth of the tumor, comparable to mice that received OT-I T cells and local intratumoral IL2 injections (FIGS. 19B and 19C). Individual follow-up data measuring tumor size is presented in FIG. 22 showing the benefits of treatment versus IL-2 injection or PBS.

To eliminate the possibility that UV irradiation exposure alone had an impact, the “Just UV” mice, whose tumors receive only UV exposure (no microfactories) were included. The use of an irrelevant plasmid (here GFP) was also tested. These two conditions did not provide any reduction in size or tumor mass as compared to the mice bearing Super2 expressing microfactories and after being activated via UV exposure (FIGS. 10A-10C). Mice where subcutaneous tumors are given on both sides, where one side received Super2 microfactories on day 3, and later received UV exposure (“treated side”) on day 6. On the contralateral side, these mice did receive Super2 microfactories on day 3 and did not receive UV exposure (“untreated side”) later. These mice received OT-I T cells on day 6 by IV injection. The second side controls for the release of Super2. “Untreated” here means with Super2 microfactories but without UV treatment.

Some mice were used, where subcutaneous tumors are given on both sides, and the mice are injected with an intra-tumoral IL-2 on day 6. The amount used was comparable to practices in the field. These mice received OT-I T cells on day 6 by IV injection. These mice control for intratumoral exposure to IL-2 that did not arise from microfactories. The mice that received Super2 microfactories but did not receive UV exposure are shown as “untreated,” as in injected with Super2 particles but untreated with the activatory UV exposure.

Some mice were injected with GFP microfactories on day 3. These mice received UV exposure on day 6. This control determines whether DNA of irrelevant plasmids may be able to elicit T cell activation or tumor clearance, e.g., through activation of TLR9. On the contralateral side, no microfactories were injected, and that side is exposed to UV exposure the same amount (“Just UV” in FIGS. 10A-10C). These mice received OT-I T cells on day 6 by IV injection. These mice demonstrated that simple UV exposure or irrelevant plasmid DNAs in the tumor environment could not augment the activated, antigen-specific OT-I T cells.

Some mice were injected with saline intratumorally on day 6 (PBS control). The amount of saline injected was the same as the volume used to deliver microfactories (˜100 μL). These mice demonstrated the aggressive rate of tumor growth of the B16 melanoma in an immunologically intact recipient.

Those mice with “untreated sides” (Super2 microfactories without UV exposure) did as well as those mice with intratumoral injection of IL2, because these mice all have OT-I T cells, so they will generally do better than the PBS control mice. In fact, the injected IL-2 side appeared to do better than the untreated side in most measurements in FIGS. 19A-19G, but the results were not statistically significant.

Summary: Thus, mice treated with Super2 microfactories dramatically enhanced tumor clearance by transferred antigen-specific T cells. Because the particles here were injected intratumorally, the UV “on switch” was not strictly necessary for treatment purposes, but permitted a legitimate comparison with the contralateral side of the mice, which received “full” microparticles but did not receive UV irradiation.

### Example 21: Mechanism of Enhanced Tumor Clearance

Objective: To better elucidate the mechanism of the enhanced tumor clearance.

Methods: Flow cytometric methods were used to examine immune cell response.

Results: Flow cytometric studies revealed that illuminated (“treated”) Super2 microfactories elicited greater expansion of tumor-infiltrating cytotoxic T cells compared to the unilluminated side (“untreated”) or controls (FIG. 19D), raising the ratio of cytotoxic effectors to helper T cells substantially (FIG. 19E). There was also a disproportionate enrichment of antigen-specific OT-I T cells on the illuminated side (FIG. 19F). Super2 microfactories drove higher expression of cytotoxin granzyme B in T cells (FIG. 19G; FIG. 11A).

Exposure to Super2 also enhanced co-expression of CD44 with Granzyme B, especially among OT-I T cells (FIGS. 11B-11C).

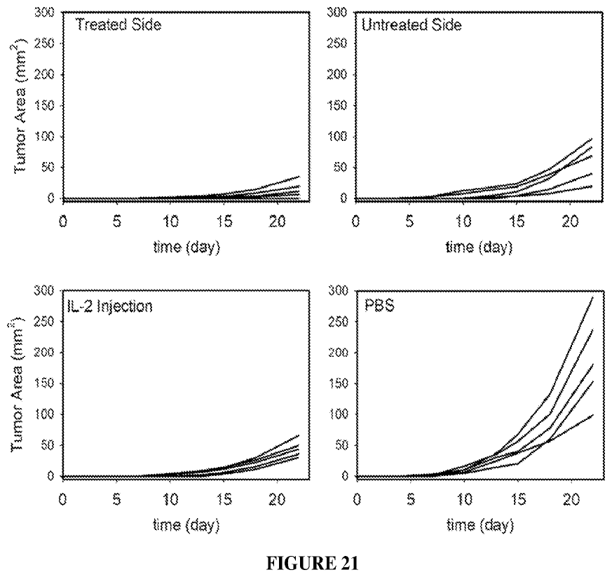

In addition, the results of a chromium release assay used to compare antigen specific (OT-I) and non-specific wild-type (WT) cytotoxicity in the presence of various IL-2 cytokine nanofactories are presented in FIG. 21. Further comparisons of treated versus untreated regions examined effector T cells and expression of granzyme B in T cells in tumors analyzed by flow cytometry (FIGS. 11A-11B).

To observe whether PD-1 based T cell exhaustion might play a role in immune evasion, the expression of PD-1 in tumor T cells was also measured (FIGS. 11D-11E). T-cell exhaustion marker PD-1 on intratumoral T cells and found comparable levels across treatments (FIGS. 11D-11E).

Summary: Together, these results provide a mechanistic explanation for the enhanced clearance of tumors by illuminated Super2 microfactories, as well as evidence supporting the notion that fast-growing tumors like B16 melanoma do not rely on PD-1-based T cell exhaustion solely for immune evasion.

Therefore, provided herein is a technological foundation for on-demand production and release of therapeutic proteins at the site of immunological action to safely augment the immune response to solid tumors. This platform can also enable control over sequential production of multiple factors during the therapeutic course, which is not trivial with competitive technologies. This platform affords the opportunity to synthesize specific factors in situ while employing controls on timing and spatial delivery that are difficult to achieve in traditional cancer therapies.

### Example 22: Targeted Microparticle Factories

Objective: To endow the microparticle factories with additional capabilities of specifically targeting T cells.

Methods: Microparticles are formed, as exemplified in Example 17 wherein methods additionally include modifying the surface of the microparticles with anti-CD3 and anti-CD28 molecules, for example but not limited to IgG, Fab, scFv, and fragments or multiples thereof, so that the particles actively interact with T cells. In certain embodiments, microparticles will be directly conjugated to T cells using covalent chemistry like NHS-EDC.

Results: Injection of a microfactory comprising a surface anti-CD3 molecule adjacent to a tumor would be expected to specifically target the microparticles to T cells in the region of the tumor, wherein the spatial and temporal regulation of cytokine production, for example controlled release of IL-2, could beneficially treat the tumor and lead to reduction or elimination of the tumor. Similarly, injection of a microfactory-T cell complex adjacent to a tumor would specifically target the T cells to a region of need at the tumor, wherein the spatial and temporal regulation of cytokine production from the microfactory, for example controlled release of IL-2, could beneficially treat the tumor and lead to reduction or elimination of the tumor.

The approach for treating cancer, for example a solid tumor, or a pre-cancerous or non-cancerous tumor, or for treating a focus of interest of an autoimmune disease or an allergic reaction or hypersensitivity reaction, a localized site of an infection or infectious disease, a localized site of an injury or other damage, a transplant or other surgical site, a blood clot, or a symptom thereof, or a combination thereof, with cytokine expressing nanoliposomes as exemplified in Examples 7, and 15-21, embodies a technological foundation for on-demand production and release of therapeutic proteins at the site of immunological action to safely augment the immune response to solid tumors. This approach affords the opportunity to synthesize specific factors in situ and deliver these factors to cells, while employing controls on timing and spatial delivery that cannot be achieved in biological systems using systemic administration, including (1) tunable initiation to eliminate the systemic toxicity of basal/continuous expression; (2) controlled release to locally focus the site of the cytokines' activity; and (3) targeting to attach the “cells” to T cells.

While certain features of the nanoliposomes, microparticles, and methods of use thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A synthetic nanoliposome comprising:
(a) a cell-free transcription and translation system; and
(b) a plasmid comprising a nucleic acid encoding a protein.

2. The synthetic nanoliposome of claim 1, further comprising:
(a) a photoactivatable-caged adenosine triphosphate (ATP).

3. The synthetic nanoliposome of claim 2, said photoactivatable-caged ATP comprising an ultraviolet (UV)-caged ATP or an infrared (IR)-caged ATP.

4. The synthetic nanoliposome of claim 1, the size of said synthetic nanoliposome comprising about 100-400 nm.

5. The synthetic nanoliposome of claim 1, the size of said plasmid comprising about 3000 bp-7000 bp.

6. The synthetic nanoliposome of claim 1, said protein comprising a therapeutic or diagnostic protein.

7. The synthetic nanoliposome of claim 1, said protein comprising a cytokine, a chemokine, a therapeutic or diagnostic antibody or fragment thereof, an antigen-binding protein, a Fc fusion protein, an anticoagulant, an enzyme, a hormone, or a thrombolytic.

8. The synthetic nanoliposome of claim 7, said cytokine comprising an interleukin (IL).

9. The synthetic nanoliposome of claim 8, said interleukin comprising an IL-2, an IL-4, an IL-10, an IL-12, or an IL-15.

10. The synthetic nanoliposome of claim 9, said IL-2 comprises an IL-2 superkine.

11. The synthetic nanoliposome of claim 10, said IL-2 superkine comprising the sequence set forth in SEQ ID NO: 10.

12. The synthetic nanoliposome of claim 10, said plasmid comprising pCellFree_G03_H9 expressing said IL-2 superkine.

13. The synthetic nanoliposome of claim 12, said plasmid having the nucleotide sequence set forth in SEQ ID NO: 9.

14. A microparticle comprising at least one synthetic nanoliposome, the at least one synthetic nanoliposome comprising:
(a) a cell-free transcription and translation system; and
(b) a plasmid comprising a nucleic acid encoding a protein.

15. The microparticle of claim 14, the synthetic nanoliposome further comprising:
(a) a photoactivatable-caged adenosine triphosphate (ATP).

16. The microparticle of claim 15, said photoactivatable-caged ATP comprising an ultraviolet (UV)-caged ATP or an infrared (IR)-caged ATP.

17. The microparticle of claim 14, the size of said synthetic nanoliposome comprising about 100-400 nm.

18. The microparticle of claim 14, the size of said plasmid comprising about 3000 bp-7000 bp.

19. The microparticle of claim 14, said protein comprising a therapeutic or diagnostic protein.

20. The microparticle of claim 14, said protein comprising a cytokine, a chemokine, a therapeutic or diagnostic antibody or fragment thereof, an antigen-binding protein, a Fc fusion protein, an anticoagulant, an enzyme, a hormone, or a thrombolytic.

21. The microparticle of claim 20, said cytokine comprising an interleukin (IL).

22. The microparticle of claim 21, said interleukin comprising an IL-2, an IL-4, an IL-10, an IL-12, or an IL-15.

23. The microparticle of claim 22, said IL-2 comprising an IL-2 superkine.

24. The synthetic nanoliposome of claim 23, said IL-2 superkine comprising the sequence set forth in SEQ ID NO: 10.

25. The synthetic nanoliposome of claim 23, said plasmid comprising pCellFree_G03_H9 expressing said IL-2 superkine.

26. The synthetic nanoliposome of claim 25, said plasmid having the sequence set forth in SEQ ID NO: 9.

27. The microparticle of claim 14, the microparticle comprising about 400 synthetic nanoliposomes.

28. The microparticle of claim 14, further comprising a superparamagnetic iron oxide nanoparticle or an upconversion nanoparticle.

29. The microparticle of claim 14, comprising:
(a) alginate; or
(b) alginate-heparin.

30. The microparticle of claim 14, the microparticle further coated in a lipid membrane.

31. The microparticle of claim 30, said lipid membrane comprising 1-palmitoyl-2-oleoyl-sn-glycero-3-phosphocholine (POPC).

32. The microparticle of claim 14, further comprising at least two types of synthetic nanoliposomes, each type of synthetic nanoliposome comprising:
(a) a cell-free transcription and translation system; and
(b) a plasmid comprising a nucleic acid encoding a protein specific to the type of synthetic nanoliposome.

33. A method of regulating an immune response at a focus of interest in a subject in need, said method comprising:
(a) administering a synthetic nanoliposome or a microparticle comprising a synthetic nanoliposome to said subject, at or adjacent to said focus of interest, said synthetic nanoliposome comprising:
(i) a cell-free transcription and translation system; and
(ii) a plasmid comprising a nucleic acid encoding a protein;
(b) expressing said therapeutic or diagnostic protein; and
(c) releasing said protein at or adjacent to said focus of interest, said regulating the immune response comprising:
(i) increasing proliferation of cytotoxic T cells;
(ii) increasing proliferation of helper T cells;
(iii) maintaining the population of helper T cells at the site of said focus of interest;
(iv) activating cytotoxic T cells at the site of said focus of interest; or
(v) any combination thereof.

34. The method of claim 33, wherein
(a) the synthetic nanoparticle further comprises:
(iii) a photoactivatable-caged adenosine triphosphate (ATP); and
(b) prior to the step of expressing said therapeutic or diagnostic protein, said site of administration is exposed to a light source to photoactivate said photoactivatable-caged ATP.

35. The method of claim 34, said photoactivatable-caged ATP comprising an ultraviolet (UV)-caged ATP or an infrared (IR)-caged ATP, and said administering comprising administering of said synthetic nanoliposome or said microparticle through a catheter comprising a UV or IR light.

36. The method of claim 33, wherein said administration comprises injection of said synthetic nanoliposome or said microparticle.

37. The method of claim 36, wherein said injection comprises subcutaneous injection.

38. The method of claim 33, said method further comprising a step of administering activated T cells to said subject.

39. The method of claim 38, said administering of said activated T cells is concomitant with administering said synthetic nanoliposome or said microparticle or is prior to or after administering said synthetic nanoliposome or said microparticle.

40. The method of claim 38, said synthetic nanoparticle further comprising a photoactivatable-caged adenosine triphosphate (ATP), said photoactivatable-caged ATP comprising an ultraviolet (UV)-caged ATP or an infrared (IR)-caged ATP, and said administering of said activated T cells is prior to or after exposing the site to UV or IR light.

41. The method of claim 33, said protein comprising a therapeutic or diagnostic protein.

42. The method of claim 33, said protein comprising a cytokine, a chemokine, a therapeutic or diagnostic antibody or fragment thereof, an antigen-binding protein, a Fc fusion protein, an anticoagulant, an enzyme, a hormone, or a thrombolytic.

43. The method of claim 42, said cytokine comprising an interleukin (IL).

44. The method of claim 43, said interleukin comprising an IL-2, an IL-4, an IL-10, an IL-12, or an IL-15.

45. The method of claim 44, said IL-2 comprising an IL-2 superkine.

46. The method of claim 45, said IL-2 superkine comprising the sequence set forth in SEQ ID NO: 10.

47. The method of claim 45, said plasmid comprising pCellFree_G03_H9 expressing said IL-2 superkine.

48. The method of claim 46, said plasmid having the sequence set forth in SEQ ID NO: 9.

49. The method of claim 33, wherein said focus of interest comprises a solid tumor.

50. The method of claim 49, wherein:
(a) said focus of interest comprises a solid tumor; and
(b) said synthetic nanoliposome or microparticle is administered at or adjacent to said solid tumor.

51. The method of claim 49, wherein said solid tumor comprises a cancerous, pre-cancerous, or non-cancerous tumor.

52. The method of claim 33, wherein said solid tumor is comprises a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

53. The method of claim 49, further comprising reducing the size of the solid tumor, eliminating said solid tumor, slowing the growth of the solid tumor, or prolonging survival of said subject, or any combination thereof.

54. The method of claim 33, wherein said focus of interest comprises:
(a) an autoimmune-targeted or symptomatic focus of an autoimmune disease;
(b) a reactive focus of an allergic reaction or hypersensitivity reaction;
(c) a focus of infection or symptoms of a localized infection or infectious disease;
(d) an injury or a site of chronic damage;
(e) a surgical site;
(f) a site of a transplanted organ, tissue, or cell; or
(g) a site of blood clot causing or at risk for causing a myocardial infarction, ischemic stroke, or pulmonary embolism.

55. The method of claim 33, wherein:
(a) the synthetic nanoliposome or microparticle is administered at or adjacent to a focus of interest comprising an autoimmune-targeted or symptomatic focus of said autoimmune disease;
(b) the synthetic nanoliposome or microparticle is administered at or adjacent to a focus of interest comprising a reactive focus of said allergic reaction or hypersensitivity reaction;
(c) the synthetic nanoliposome or microparticle is administered at or adjacent to a focus of interest comprising a focus of infection or symptoms;
(d) the synthetic nanoliposome or microparticle is administered at or adjacent to a focus of interest comprising the injury or the site of chronic damage;
(e) the synthetic nanoliposome or microparticle is administered at or adjacent to a focus of interest comprising the surgical site;
(f) the synthetic nanoliposome or microparticle is administered at or adjacent to a focus of interest comprising a transplant site; or
(g) the synthetic nanoliposome or microparticle is administered at or adjacent to a focus of interest comprising the site of the blood clot.

56. The method of claim 33, wherein said focus of interest comprises a blood clot causing or at risk for causing a myocardial infarction, an ischemic stroke, or a pulmonary embolism, and the synthetic nanoliposome or microparticle is administered at or adjacent to the site of the blood clot together with angioplasty or another clot removal treatment.

57. The method of claim 33, further comprising:
(a) administering two of more types of microparticles, each type of microparticle comprising a synthetic nanoliposome comprising:
(i) a cell-free transcription and translation system; and
(ii) a plasmid comprising a nucleic acid encoding a protein specific to the type of microparticle; or
(b) administering at least one type of microparticle, each microparticle further comprising at least two types of synthetic nanoliposomes, each type of synthetic nanoliposome comprising:
(i) a cell-free transcription and translation system; and
(ii) a plasmid comprising a nucleic acid encoding a protein specific to the type of synthetic nanoliposome.

58. A method of treating a disease or medical condition, or of alleviating symptoms thereof, at a focus of interest in a subject in need, said method comprising:
(a) administering a synthetic nanoliposome or a microparticle comprising a synthetic nanoliposome to said subject, at or adjacent to a focus of interest, said synthetic nanoliposome comprising:
(i) a cell-free transcription and translation system; and
(ii) a plasmid comprising a nucleic acid encoding a therapeutic or diagnostic protein;
(b) expressing said therapeutic or diagnostic protein;
(c) releasing said protein at or adjacent to said focus of interest.

59. The method of claim 58, wherein:
(a) said synthetic nanoliposome further comprises:
(iii) a photoactivatable-caged adenosine triphosphate (ATP); and
(b) prior to the step of expressing said therapeutic or diagnostic protein, the site of administration is exposed to a light source to photoactivate said photoactivatable-caged ATP.

60. The method of claim 58, wherein:
(a) the disease or medical condition comprises a solid tumor; and
(b) the synthetic nanoliposomes or microparticles are administered at or adjacent to a focus of interest comprising said solid tumor.

61. The method of claim 60, wherein said solid tumor comprises a cancerous, pre-cancerous, or non-cancerous tumor.

62. The method of claim 60, wherein said solid tumor is comprises a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma.

63. The method of claim 60, wherein said treating reduces the size of the solid tumor, eliminates said solid tumor, slows the growth of the solid tumor, or prolongs survival of said subject, or any combination thereof.

64. The method of claim 58, wherein:
(a) the disease or medical condition comprises an autoimmune disease, and the synthetic nanoliposome or microparticle is administered at or adjacent to a focus of interest comprising an autoimmune-targeted or symptomatic focus of said autoimmune disease;
(b) the disease or medical condition comprises an allergic reaction or hypersensitivity reaction, and the synthetic nanoliposome or microparticle is administered at or adjacent to a focus of interest comprising a reactive focus of said allergic reaction or hypersensitivity reaction;
(c) the disease or medical condition comprises a localized infection or an infectious disease, and the synthetic nanoliposome or microparticle is administered at or adjacent to a focus of interest comprising a focus of infection or symptoms;
(d) the disease or medical condition comprises an injury or a site of chronic damage, and the synthetic nanoliposome or microparticle is administered at or adjacent to a focus of interest comprising the injury or the site of chronic damage;
(e) the disease or medical condition comprises a surgical site, and the synthetic nanoliposome or microparticle is administered at or adjacent to a focus of interest comprising the surgical site;
(f) the disease or medical condition comprises a transplanted organ, tissue, or cell, and the synthetic nanoliposome or microparticle is administered at or adjacent to a focus of interest comprising a transplant site; or
(g) the disease or medical condition comprises a blood clot causing or at risk for causing a myocardial infarction, an ischemic stroke, or a pulmonary embolism, and the synthetic nanoliposome or microparticle is administered at or adjacent to a focus of interest comprising the site of the blood clot.

65. The method of claim 58, wherein said disease or medical condition comprises a blood clot causing or at risk for causing a myocardial infarction, an ischemic stroke, or a pulmonary embolism, and the synthetic nanoliposome or microparticle is administered at or adjacent to a focus of interest comprising the site of the blood clot together with angioplasty or another clot removal treatment.

66. The method of claim 58, wherein said treating:
(a) reduces or eliminates inflammation or another symptom of said autoimmune-targeted or symptomatic focus of said autoimmune disease, prolongs survival of said subject, or any combination thereof;
(b) reduces or eliminates inflammation or another symptom of allergic reaction or hypersensitivity reaction at said reactive focus of said allergic reaction or hypersensitivity reaction, prolongs survival of said subject, or any combination thereof;
(c) reduces or eliminates infection or symptoms at said focus of infection or symptoms of said localized infection or infectious disease, prolongs survival of said subject, or any combination thereof;
(d) reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof;
(e) reduces, eliminates, inhibits, or prevents structural, organ, tissue, or cell damage, inflammation, infection, or another symptom at said surgical site, improves structural, organ, tissue, or cell function at said surgical site, improves mobility of said subject, prolongs survival of said subject, or any combination thereof;
(f) reduces, eliminates, inhibits or prevents transplanted organ, tissue, or cell damage or rejection, inflammation, infection or another symptom at said transplant site, improves mobility of said subject, prolongs survival of said transplanted organ, tissue, or cell, prolongs survival of said subject, or any combination thereof; or
(g) reduces or eliminates said blood clot causing or at risk for causing said myocardial infarction, said ischemic stroke, or said pulmonary embolism in said subject, improves function or survival of a heart, brain, or lung organ, tissue, or cell in said subject, reduces damage to a heart, brain, or lung organ, tissue, or cell in said subject, prolongs survival of a heart, brain, or lung organ, tissue, or cell in said subject, prolongs survival of said subject, or any combination thereof.

67. The method of claim 58, further comprising:
(a) administering two of more types of microparticles, each type of microparticle comprising a synthetic nanoliposome comprising:
(i) a cell-free transcription and translation system; and
(ii) a plasmid comprising a nucleic acid encoding a protein specific to the type of microparticle; or
(b) administering at least one type of microparticle, each microparticle further comprising at least two types of synthetic nanoliposomes, each type of synthetic nanoliposome comprising:
(i) a cell-free transcription and translation system; and
(ii) a plasmid comprising a nucleic acid encoding a protein specific to the type of synthetic nanoliposome.

68. A method of making a synthetic nanoliposome comprising a cell-free transcription and translation system; a plasmid comprising a nucleic acid encoding a protein; and a photoactivatable-caged adenosine triphosphate (ATP), said method comprising:
(a) providing a lipid solution by:
(i) combining 1,2-dimyristoyl-sn-glycero-3-phosphocholine (DMPC), 1-palmitoyl-2-oleoyl-phosphatidylcholine (POPC), and cholesterol, to yield a lipid mixture;
(ii) drying said lipid mixture to form a solid lipid mixture;
(b) providing a caged plasmid/cell-free transcription-translation mixture comprising:
(i) a cell-free transcription and translation system; and
(ii) a caged plasmid comprising a nucleic acid encoding a protein;
(c) combining said solid lipid mixture and said caged plasmid/cell-free transcription-translation mixture to yield said synthetic nanoliposome.

69. The method of claim 68, said caged plasmid/cell-free transcription-translation mixture further comprising a photoactivatable-caged adenosine triphosphate (ATP).

70. The method of claim 68, the combining step comprising:
(a) dissolving said solid lipid mixture in a volatile solvent to form a lipid solution;
(b) providing a microfluidic device comprising a plurality of channels comprising a center channel and one or more flanking channels;
(c) injecting said caged plasmid/cell-free transcription-translation mixture through said center channel of said microfluidic device while simultaneously injecting said lipid solution through one or more channels flanking said center channel of said microfluidic device and optionally simultaneously injecting water or buffer through one or more additional channels in said microfluidic device to yield said synthetic nanoliposome.

71. The method of claim 68, the combining step comprising:
(a) warming said solid lipid mixture to a temperature of about 40 degrees C. to about 90 degrees C.;
(b) mixing said caged plasmid/cell-free transcription-translation mixture with said warmed solid lipid mixture;
(c) isolating at least one liposome from the mixture, said at least one liposome encapsulating said caged plasmid/cell-free transcription-translation mixture;
(d) agitating said at least one liposome in solution;
(e) extruding said liposome solution through a porous membrane to provide at least one synthetic nanoliposome comprising said caged plasmid/cell-free transcription-translation mixture to yield said synthetic nanoliposome.

72. The method of claim 68, further comprising:
(a) providing an alginate solution or an alginate-heparin conjugate solution;
(b) mixing said alginate solution or said alginate-heparin conjugate solution with said synthetic nanoliposome to yield an aqueous phase;
(c) mixing the aqueous phase with a surfactant in a continuous phase, said continuous phase comprising a surfactant and a non-aqueous solvent to yield a microparticle comprising said nanoliposome;
(d) cross-linking said microparticle with an ionic solution; and
(e) isolating said microparticle.

73. The method of claim 68, said volatile solvent comprising acetone, chloroform, isopropanol, or methanol.

74. The method of claim 68, said caged plasmid caged with (1-(4,5-dimethoxy-2-nitrophenyl) ethyl ester (DMNPE).
